SEQUENCE LISTING (160 sequences)

SEQ ID NO: 1 - protein, 12 aa, Artificial Sequence
  Amino acid sequence of isolated G12D Ab1 and G12D Ab2 peptides
GADGVGKSALTI

SEQ ID NO: 2 - protein, 12 aa, Artificial Sequence
  Amino acid sequence of isolated G13D Ab1 and G13D Ab2 peptides
GAGDVGKSALTI

SEQ ID NO: 3 - protein, 13 aa, Artificial Sequence
  Amino acid sequence of isolated G12D Ab1 peptide, including additional modifications present for the purposes of antibody generation
  MISC_FEATURE (1)..(13): The peptide is amidated at the C-terminus and is acetylated at the N-terminus
GADGVGKSALTIC

SEQ ID NO: 4 - protein, 13 aa, Artificial Sequence
  Amino acid sequence of isolated G12D Ab2 peptide, including additional modifications present for the purposes of antibody generation
  MISC_FEATURE (1)..(13): The peptide is amidated at the C-terminus
CGADGVGKSALTI

SEQ ID NO: 5 - protein, 13 aa, Artificial Sequence
  Amino acid sequence of isolated G13D Ab1 peptide, including additional modifications present for the purposes of antibody generation
  MISC_FEATURE (1)..(13): The peptide is amidated at the C-terminus
CGAGDVGKSALTI

SEQ ID NO: 6 - protein, 13 aa, Artificial Sequence
  Amino acid sequence of isolated G13D Ab2 peptide, including additional modifications present for the purposes of antibody generation
  MISC_FEATURE (1)..(13): The peptide is amidated at the C-terminus and is acetylated at the N-terminus
GAGDVGKSALTIC

SEQ ID NO: 7 - protein, 189 aa, Human [sequence omitted]

SEQ ID NO: 8 - protein, 189 aa, Human
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 9 - protein, 189 aa, Human
MTEYKLVVVGAGDVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 10 - protein, 188 aa, Human [sequence omitted]

SEQ ID NO: 11 - protein, 188 aa, Human [sequence omitted]

SEQ ID NO: 12 - protein, 188 aa, Human [sequence omitted]

SEQ ID NO: 13 - protein, 189 aa, Artificial Sequence
  Amino acid sequence of a human G12X mutant KRAS (Isoform 2A)
  MISC_FEATURE (12)..(12): Asp, Val, Cys, Ala, Ser or Arg
MTEYKLVVVGAXGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 14 - protein, 189 aa, Artificial Sequence
  Amino acid sequence of human G13X mutant KRAS (Isoform 2A)
  MISC_FEATURE (13)..(13): Asp or Cys
MTEYKLVVVGAGXVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 15 - protein, 188 aa, Artificial Sequence
  Amino acid sequence of a human G12X mutant KRAS (Isoform 2B)
  MISC_FEATURE (12)..(12): Asp, Val, Cys, Ala, Ser or Arg
MTEYKLVVVGAXGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 16 - protein, 188 aa, Artificial Sequence
  Amino acid sequence of human G13X mutant KRAS (Isoform 2B)
  MISC_FEATURE (13)..(13): Asp or Cys
MTEYKLVVVGAGXVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 17 - protein, 12 aa, Artificial Sequence
  Amino acid sequence of an isolated G12X peptide
  MISC_FEATURE (3)..(3): Asp, Val, Cys, Ala, Ser or Arg
GAXGVGKSALTI

SEQ ID NO: 18 - protein, 12 aa, Artificial Sequence
  Amino acid sequence of isolated G13X peptide
  MISC_FEATURE (4)..(4): Asp or Cys
GAGXVGKSALTI

SEQ ID NO: 19 - protein, 12 aa, Artificial Sequence
  Amino acid sequence of an isolated G12X peptide
  MISC_FEATURE (3)..(3): Asp, Val, Cys, Ala or Arg
GAXGVGKSALTI

SEQ ID NO: 20 - protein, 12 aa, Artificial Sequence
  Amino acid sequence of an isolated G12X peptide
  MISC_FEATURE (3)..(3): Asp, Cys, Ala, Ser or Arg
GAXGVGKSALTI

SEQ ID NO: 21 - protein, 12 aa, Artificial Sequence
  Amino acid sequence of an isolated G12X peptide
  MISC_FEATURE (3)..(3): Asp, Cys, Ala or Arg
GAXGVGKSALTI

SEQ ID NO: 22 - protein, 189 aa, Artificial Sequence; identical to SEQ ID NO: 13
  Amino acid sequence of a human G12X mutant KRAS (Isoform 2A)
  MISC_FEATURE (12)..(12): Asp, Val, Cys, Ala or Arg

SEQ ID NO: 23 - protein, 188 aa, Artificial Sequence; identical to SEQ ID NO: 15
  Amino acid sequence of a human G12X mutant KRAS (Isoform 2B)
  MISC_FEATURE (12)..(12): Asp, Val, Cys, Ala or Arg

SEQ ID NO: 24 - protein, 189 aa, Artificial Sequence; identical to SEQ ID NO: 13
  Amino acid sequence of a human G12X mutant KRAS (Isoform 2A)
  MISC_FEATURE (12)..(12): Asp, Cys, Ala, Ser or Arg

SEQ ID NO: 25 - protein, 188 aa, Artificial Sequence; identical to SEQ ID NO: 15
  Amino acid sequence of a human G12X mutant KRAS (Isoform 2B)
  MISC_FEATURE (12)..(12): Asp, Cys, Ala, Ser or Arg

SEQ ID NO: 26 - protein, 189 aa, Artificial Sequence; identical to SEQ ID NO: 13
  Amino acid sequence of a human G12X mutant KRAS (Isoform 2A)
  MISC_FEATURE (12)..(12): Asp, Cys, Ala or Arg

SEQ ID NO: 27 - protein, 188 aa, Artificial Sequence; identical to SEQ ID NO: 15
  Amino acid sequence of a human G12X mutant KRAS (Isoform 2B)
  MISC_FEATURE (12)..(12): Asp, Cys, Ala or Arg

SEQ ID NO: 28 - DNA, 351 bp, Mouse
gaggtccagctgcaacagtctggacctgaactggtgaagcctggggcttcagtgaagatgtcctgcaagacttctggatacacattcactgactacaccatgcactgggtgaagcagagccatggaaagagccttgagtggattggatatattaaccctaacaatggtggaacttactacaaccagaggttcaagggcaaggccacattgactgtaagcaggtcctccagcacagcctacatagagctccgcagcctgacatcggacgattctgcagtctattactgtgcacgaggggattactacggtggtggctactggggccaaggcaccacgctcagagtctcctca

SEQ ID NO: 29 - DNA, 318 bp, Mouse
gagaatgttctcacccagtctccagcaatcatgtctgcatctccaggggaaaaggtcaccatgacctgcagtgccggctcaagtgtaagttacatgcactggtatcagcagaagtcaagcacctcccccaaactctggatttatgacacatccaaactggcttctggagtcccgggtcgcttcagtggcagtgggtctggaaactcttactctctcacgatcagcagcatggaggctgaagatgttgccacttattactgttttcaggggagtgggtacccactcacgttcggtgatgggaccaagctggagctgaaa

SEQ ID NO: 30 - protein, 117 aa, Mouse
EVQLQQSGPELVKPGASVKMSCKTSGYTFTDYTMHWVKQSHGKSLEWIGYINPNNGGTYYNQRFKGKATLTVSRSSSTAYIELRSLTSDDSAVYYCARGDYYGGGYWGQGTTLRVSS

SEQ ID NO: 31 - protein, 106 aa, Mouse
ENVLTQSPAIMSASPGEKVTMTCSAGSSVSYMHWYQQKSSTSPKLWIYDTSKLASGVPGRFSGSGSGNSYSLTISSMEAEDVATYYCFQGSGYPLTFGDGTKLELK

SEQ ID NO: 32 - protein, 5 aa, Mouse
DYTMH

SEQ ID NO: 33 - protein, 17 aa, Mouse
YINPNNGGTYYNQRFKG

SEQ ID NO: 34 - protein, 8 aa, Mouse
GDYYGGGY

SEQ ID NO: 35 - protein, 10 aa, Mouse
SAGSSVSYMH

SEQ ID NO: 36 - protein, 7 aa, Mouse
DTSKLAS

SEQ ID NO: 37 - protein, 9 aa, Mouse
FQGSGYPLT

SEQ ID NO: 38 - protein, 30 aa, Mouse
EVQLQQSGPELVKPGASVKMSCKTSGYTFT

SEQ ID NO: 39 - protein, 14 aa, Mouse
WVKQSHGKSLEWIG

SEQ ID NO: 40 - protein, 32 aa, Mouse
KATLTVSRSSSTAYIELRSLTSDDSAVYYCAR

SEQ ID NO: 41 - protein, 11 aa, Mouse
WGQGTTLRVSS

SEQ ID NO: 42 - protein, 23 aa, Mouse
ENVLTQSPAIMSASPGEKVTMTC

SEQ ID NO: 43 - protein, 15 aa, Mouse
WYQQKSSTSPKLWIY

SEQ ID NO: 44 - protein, 32 aa, Mouse
GVPGRFSGSGSGNSYSLTISSMEAEDVATYYC

SEQ ID NO: 45 - protein, 10 aa, Mouse
FGDGTKLELK

SEQ ID NO: 46 - protein, 19 aa, Mouse
MGWSWIFLFLLSETAGVLS

SEQ ID NO: 47 - protein, 22 aa, Mouse
MDFQVQIFSFLLISASVIMSRG

SEQ ID NO: 48 - DNA, 351 bp, Mouse
gaggtccagctgcaacagtcaggacctgagcgggtgaagcctggggcttcagtgaagatgtcctgcaaggcttctggatacacattcactgactacagcatgcactgggtgaagcagagccatggaaagagccttgaatggattggatatattaacccgaacaatggtggtgctagctacaaccagaagttcaagggcaaggccacattgactgtaaacaagtcctccagcacagcctacatggagctccgcagcctgacatcggaggattctgcagtctattactgtgcaagaggggattactacggtggtggctactggggccaaggcaccactctcacagtctcctca

SEQ ID NO: 49 - DNA, 318 bp, Mouse
gaaaatgttctcacccagtctccagcaatcatgtctgcatctccaggggaaaaggtcaccatgacctgcagtgccagctcaagtgtaagttacatgcactggtaccagcagaagtcaagcacctcccccaaactctggatttatgacacatccaaagtggcttctggagtcccaggtcgcttcagtggcagtgggtctggaaactcttactctctcacgatcaacagcatggaggctgaagatgttgccacttattactgttttcaggggagtgggtacccactcacgttcggtgctgggaccaagctggagctgaaa

SEQ ID NO: 50 - protein, 117 aa, Mouse
EVQLQQSGPERVKPGASVKMSCKASGYTFTDYSMHWVKQSHGKSLEWIGYINPNNGGASYNQKFKGKATLTVNKSSSTAYMELRSLTSEDSAVYYCARGDYYGGGYWGQGTTLTVSS

SEQ ID NO: 51 - protein, 106 aa, Mouse
ENVLTQSPAIMSASPGEKVTMTCSASSSVSYMHWYQQKSSTSPKLWIYDTSKVASGVPGRFSGSGSGNSYSLTINSMEAEDVATYYCFQGSGYPLTFGAGTKLELK

SEQ ID NO: 52 - protein, 5 aa, Mouse
DYSMH

SEQ ID NO: 53 - protein, 17 aa, Mouse
YINPNNGGASYNQKFKG

SEQ ID NO: 54 - protein, 8 aa, Mouse
GDYYGGGY

SEQ ID NO: 55 - protein, 10 aa, Mouse
SASSSVSYMH

SEQ ID NO: 56 - protein, 7 aa, Mouse
DTSKVAS

SEQ ID NO: 57 - protein, 9 aa, Mouse
FQGSGYPLT

SEQ ID NO: 58 - protein, 30 aa, Mouse
EVQLQQSGPERVKPGASVKMSCKASGYTFT

SEQ ID NO: 59 - protein, 14 aa, Mouse
WVKQSHGKSLEWIG

SEQ ID NO: 60 - protein, 32 aa, Mouse
KATLTVNKSSSTAYMELRSLTSEDSAVYYCAR

SEQ ID NO: 61 - protein, 11 aa, Mouse
WGQGTTLTVSS

SEQ ID NO: 62 - protein, 23 aa, Mouse
ENVLTQSPAIMSASPGEKVTMTC

SEQ ID NO: 63 - protein, 15 aa, Mouse
WYQQKSSTSPKLWIY

SEQ ID NO: 64 - protein, 32 aa, Mouse
GVPGRFSGSGSGNSYSLTINSMEAEDVATYYC

SEQ ID NO: 65 - protein, 10 aa, Mouse
FGAGTKLELK

SEQ ID NO: 66 - protein, 19 aa, Mouse
MGWSWIFLFLLSETAGVLS

SEQ ID NO: 67 - protein, 22 aa, Mouse
MDFQVQIFSFLLISASVIMSRG

SEQ ID NO: 68 - DNA, 342 bp, Mouse
caggtccaactgcagcagcctggggctgagtttgtgaagcctggggcttcagtgaagctgtcctgcaaggcttctggctacaccttcacccgctactggatgcactgggtgaagcagaggcctggacgaggccttgagtggattggaaggattgatcctaatagtggtggtactacgttcaatgagaagttcaagagcaaggccacactgactgtagacaaaccctccagcacagcctacatgcagctcagcagcctgacatctgaggactctgcggtctattattgtgcaagagaggtcgggggctactggggccaaggcaccactctcacagtctcctca

SEQ ID NO: 69 - DNA, 318 bp, Mouse
gaaaatgttctcacccagtctccagcaatcatgtctgcatctccaggggaaaaggtcaccatgacctgcagtgccgggtcaagtgtagattacatgcactggtaccagcagaagtcaagcacctcccccaaactctggatttatgacacatccaagctggcttctggagtcccaggtcgcttcagtggcagtgggtctggaaactcttattctctcacgatcagcagcatggaggctgaagatgttgccacttattactgttttcaggggagtgggtacccactcacgttcggtgctgggaccaagctggagctgaga

SEQ ID NO: 70 - protein, 114 aa, Mouse
QVQLQQPGAEFVKPGASVKLSCKASGYTFTRYWMHWVKQRPGRGLEWIGRIDPNSGGTTFNEKFKSKATLTVDKPSSTAYMQLSSLTSEDSAVYYCAREVGGYWGQGTTLTVSS

SEQ ID NO: 71 - protein, 106 aa, Mouse
ENVLTQSPAIMSASPGEKVTMTCSAGSSVDYMHWYQQKSSTSPKLWIYDTSKLASGVPGRFSGSGSGNSYSLTISSMEAEDVATYYCFQGSGYPLTFGAGTKLELR

SEQ ID NO: 72 - protein, 5 aa, Mouse
RYWMH

SEQ ID NO: 73 - protein, 17 aa, Mouse
RIDPNSGGTTFNEKFKS

SEQ ID NO: 74 - protein, 5 aa, Mouse
EVGGY

SEQ ID NO: 75 - protein, 10 aa, Mouse
SAGSSVDYMH

SEQ ID NO: 76 - protein, 7 aa, Mouse
DTSKLAS

SEQ ID NO: 77 - protein, 9 aa, Mouse
FQGSGYPLT

SEQ ID NO: 78 - protein, 30 aa, Mouse
QVQLQQPGAEFVKPGASVKLSCKASGYTFT

SEQ ID NO: 79 - protein, 14 aa, Mouse
WVKQRPGRGLEWIG

SEQ ID NO: 80 - protein, 32 aa, Mouse
KATLTVDKPSSTAYMQLSSLTSEDSAVYYCAR

SEQ ID NO: 81 - protein, 11 aa, Mouse
WGQGTTLTVSS

SEQ ID NO: 82 - protein, 23 aa, Mouse
ENVLTQSPAIMSASPGEKVTMTC

SEQ ID NO: 83 - protein, 15 aa, Mouse
WYQQKSSTSPKLWIY

SEQ ID NO: 84 - protein, 32 aa, Mouse; identical to SEQ ID NO: 44

SEQ ID NO: 85 - protein, 10 aa, Mouse
FGAGTKLELR

SEQ ID NO: 86 - protein, 19 aa, Mouse
MGWSCIMLFLAATATGVHS

SEQ ID NO: 87 - protein, 22 aa, Mouse
MDFQVQIFSFLLISASVIMSRG

SEQ ID NO: 88 - DNA, 357 bp, Mouse
caggttactctgaaagagtctggccctgggatattgcagccctcccagaccctcagtctgacttgttctttctctgggttttcactgagcacttttggtatgggtgtaggctggattcgtcagccttcagggaagggtctggagtggctggcacacatttggtgggatgatgataagaactataacccagccctgaagagtcggctcacaatctccaaggatacctccaaaaaccaggttttcttcaagatcgccaatgtggacactgcagatactgccacatactactgtgttcaactttctactatgggagacggggggtactggggccaagggactctggtcactgtctctgca

SEQ ID NO: 89 - DNA, 321 bp, Mouse
aacattgtaatgacccaatctcccaaatccatgtccatgtcagtaggagagagggtcaccttgagctgcaaggccagtgagaatgtgggtacttatgtatcctggtatcaacagaaaccagggcagtctcctaaattgttgatatacggggcatccaaccggtacactggggtccccgatcgcttcacaggcagtggatctggaacagatttcattctgaccatcagcagtgtgcaggctgaagaccttgcagattatcactgtggacagagttacaactatccattcacgttcggctcggggacaaagttggaaataaaa

SEQ ID NO: 90 - protein, 119 aa, Mouse
QVTLKESGPGILQPSQTLSLTCSFSGFSLSTFGMGVGWIRQPSGKGLEWLAHIWWDDDKNYNPALKSRLTISKDTSKNQVFFKIANVDTADTATYYCVQLSTMGDGGYWGQGTLVTVSA

SEQ ID NO: 91 - protein, 107 aa, Mouse
NIVMTQSPKSMSMSVGERVTLSCKASENVGTYVSWYQQKPGQSPKLLIYGASNRYTGVPDRFTGSGSGTDFILTISSVQAEDLADYHCGQSYNYPFTFGSGTKLEIK

SEQ ID NO: 92 - protein, 7 aa, Mouse
TFGMGVG

SEQ ID NO: 93 - protein, 16 aa, Mouse
HIWWDDDKNYNPALKS

SEQ ID NO: 94 - protein, 9 aa, Mouse
LSTMGDGGY

SEQ ID NO: 95 - protein, 11 aa, Mouse
KASENVGTYVS

SEQ ID NO: 96 - protein, 7 aa, Mouse
GASNRYT

SEQ ID NO: 97 - protein, 9 aa, Mouse
GQSYNYPFT

SEQ ID NO: 98 - protein, 30 aa, Mouse
QVTLKESGPGILQPSQTLSLTCSFSGFSLS

SEQ ID NO: 99 - protein, 14 aa, Mouse
WIRQPSGKGLEWLA

SEQ ID NO: 100 - protein, 32 aa, Mouse
RLTISKDTSKNQVFFKIANVDTADTATYYCVQ

SEQ ID NO: 101 - protein, 11 aa, Mouse
WGQGTLVTVSA

SEQ ID NO: 102 - protein, 23 aa, Mouse
NIVMTQSPKSMSMSVGERVTLSC

SEQ ID NO: 103 - protein, 15 aa, Mouse
WYQQKPGQSPKLLIY

SEQ ID NO: 104 - protein, 32 aa, Mouse
GVPDRFTGSGSGTDFILTISSVQAEDLADYHC

SEQ ID NO: 105 - protein, 10 aa, Mouse
FGSGTKLEIK

SEQ ID NO: 106 - protein, 19 aa, Mouse
MGRLTSSFLLLIVPAYVLS

SEQ ID NO: 107 - protein, 20 aa, Mouse
MESQTLVFISILLWLYGADG

SEQ ID NO: 108 - DNA, 366 bp, Mouse
caggttactctgaaagagtctggccctgggatattgcagccctcccagaccctcagtctgacttgttctttctctgggttttcactgagcacttttggtgtgggtgtagcctggattcgtcagccttcagggaagggtctggagtggctggcacacatttggtgggatgatgataagaactatgacccagccctgaagagtcggctcacaatctccaaggatacctccaaaaaccaggttttcctcaagatcgccaatgtggacactacagattctgccacatactactgtgctcgaataggatctactttgattacgccaggggatgcttcctggggccaggggactctggtcactgtctctgca

SEQ ID NO: 109 - DNA, 321 bp, Mouse
aacattgttatgacccaatctcccaaatccatgtccatgtcagtaggagagagggtcaccttgagctgcaaggccagtgagaatgtgggtacttttgtatcctggtatcaacagaaaccagaccagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagagttacagctatccatacacgttcggaggggggaccaagctggaaatcaaa

SEQ ID NO: 110 - protein, 122 aa, Mouse
QVTLKESGPGILQPSQTLSLTCSFSGFSLSTFGVGVAWIRQPSGKGLEWLAHIWWDDDKNYDPALKSRLTISKDTSKNQVFLKIANVDTTDSATYYCARIGSTLITPGDASWGQGTLVTVSA

SEQ ID NO: 111 - protein, 107 aa, Mouse
NIVMTQSPKSMSMSVGERVTLSCKASENVGTFVSWYQQKPDQSPKLLIYGASNRYTGVPDRFTGSGSATDFTLTISSVQAEDLADYHCGQSYSYPYTFGGGTKLEIK

SEQ ID NO: 112 - protein, 7 aa, Mouse
TFGVGVA

SEQ ID NO: 113 - protein, 16 aa, Mouse
HIWWDDDKNYDPALKS

SEQ ID NO: 114 - protein, 12 aa, Mouse
IGSTLITPGDAS

SEQ ID NO: 115 - protein, 11 aa, Mouse
KASENVGTFVS

SEQ ID NO: 116 - protein, 7 aa, Mouse
GASNRYT

SEQ ID NO: 117 - protein, 9 aa, Mouse
GQSYSYPYT

SEQ ID NO: 118 - protein, 30 aa, Mouse; identical to SEQ ID NO: 98

SEQ ID NO: 119 - protein, 14 aa, Mouse
WIRQPSGKGLEWLA

SEQ ID NO: 120 - protein, 32 aa, Mouse
RLTISKDTSKNQVFLKIANVDTTDSATYYCAR

SEQ ID NO: 121 - protein, 11 aa, Mouse
WGQGTLVTVSA

SEQ ID NO: 122 - protein, 23 aa, Mouse
NIVMTQSPKSMSMSVGERVTLSC

SEQ ID NO: 123 - protein, 15 aa, Mouse
WYQQKPDQSPKLLIY

SEQ ID NO: 124 - protein, 32 aa, Mouse
GVPDRFTGSGSATDFTLTISSVQAEDLADYHC

SEQ ID NO: 125 - protein, 10 aa, Mouse
FGGGTKLEIK

SEQ ID NO: 126 - protein, 19 aa, Mouse
MGRLTSSFLLLIVPAYVLS

SEQ ID NO: 127 - protein, 20 aa, Mouse
MESQTLVFISILLWLFGADG

SEQ ID NO: 128 - DNA, 366 bp, Mouse; identical to SEQ ID NO: 108

SEQ ID NO: 129 - DNA, 327 bp, Mouse
gatattgtactaactcagtctccagccaccctgtctgtgactccaggagatagagtcagtctttcctgcagggccagtcaaagtattagcgactacctacactggtatcaacaaaaatcacatgagtctccaaggcttctcatcaagtttgcttcccagtccatctctgggatcccctccaggttcagtggcagtggatcagggacagatttcactctcagtatcaacagtgtggagactgaagattttggaatgtatttctgtcaacagagtaacaactggcctcacatgtacacgttcggaggggggaccaagctggaaataaaa

SEQ ID NO: 130 - protein, 122 aa, Mouse; identical to SEQ ID NO: 110

SEQ ID NO: 131 - protein, 109 aa, Mouse
DIVLTQSPATLSVTPGDRVSLSCRASQSISDYLHWYQQKSHESPRLLIKFASQSISGIPSRFSGSGSGTDFTLSINSVETEDFGMYFCQQSNNWPHMYTFGGGTKLEIK

SEQ ID NO: 132 - protein, 7 aa, Mouse
TFGVGVA

SEQ ID NO: 133 - protein, 16 aa, Mouse
HIWWDDDKNYDPALKS

SEQ ID NO: 134 - protein, 12 aa, Mouse
IGSTLITPGDAS

SEQ ID NO: 135 - protein, 11 aa, Mouse
RASQSISDYLH

SEQ ID NO: 136 - protein, 7 aa, Mouse
FASQSIS

SEQ ID NO: 137 - protein, 11 aa, Mouse
QQSNNWPHMYT

SEQ ID NO: 138 - protein, 30 aa, Mouse; identical to SEQ ID NO: 98

SEQ ID NO: 139 - protein, 14 aa, Mouse
WIRQPSGKGLEWLA

SEQ ID NO: 140 - protein, 32 aa, Mouse; identical to SEQ ID NO: 120

SEQ ID NO: 141 - protein, 11 aa, Mouse
WGQGTLVTVSA

SEQ ID NO: 142 - protein, 23 aa, Mouse
DIVLTQSPATLSVTPGDRVSLSC

SEQ ID NO: 143 - protein, 15 aa, Mouse
WYQQKSHESPRLLIK

SEQ ID NO: 144 - protein, 32 aa, Mouse
GIPSRFSGSGSGTDFTLSINSVETEDFGMYFC

SEQ ID NO: 145 - protein, 10 aa, Mouse
FGGGTKLEIK

SEQ ID NO: 146 - protein, 19 aa, Mouse
MGRLTSSFLLLIVPAYVLS

SEQ ID NO: 147 - protein, 20 aa, Mouse
MVFTPQILGLMLFWISASRG

SEQ ID NO: 148 - protein, 5 aa, Artificial Sequence
  Consensus Heavy CDR1
  MISC_FEATURE (1)..(1): any amino acid
  MISC_FEATURE (3)..(3): any amino acid
XYXMH

SEQ ID NO: 149 - protein, 5 aa, Artificial Sequence
  Consensus Heavy CDR1
  MISC_FEATURE (1)..(1): Asp or Arg
  MISC_FEATURE (3)..(3): Ser, Thr or Trp
XYXMH

SEQ ID NO: 150 - protein, 17 aa, Artificial Sequence
  Consensus Heavy CDR2
  MISC_FEATURE (1)..(1): any amino acid
  MISC_FEATURE (3)..(3): any amino acid
  MISC_FEATURE (6)..(6): any amino acid
  MISC_FEATURE (9)..(9): any amino acid
  MISC_FEATURE (10)..(10): any amino acid
  MISC_FEATURE (11)..(11): any amino acid
  MISC_FEATURE (13)..(13): any amino acid
  MISC_FEATURE (14)..(14): any amino acid
  MISC_FEATURE (17)..(17): any amino acid
XIXPNXGGXXXNXXFKX

SEQ ID NO: 151 - protein, 17 aa, Artificial Sequence
  Consensus Heavy CDR2
  MISC_FEATURE (1)..(1): Tyr or Arg (preferably Tyr)
  MISC_FEATURE (3)..(3): Asn or Asp (preferably Asn)
  MISC_FEATURE (6)..(6): Asn or Ser (preferably Asn)
  MISC_FEATURE (9)..(9): Ala or Thr
  MISC_FEATURE (10)..(10): Ser or Tyr or Thr
  MISC_FEATURE (11)..(11): Tyr or Phe (preferably Tyr)
  MISC_FEATURE (13)..(13): Gln or Glu (preferably Gln)
  MISC_FEATURE (14)..(14): Lys or Arg
  MISC_FEATURE (17)..(17): Gly or Ser (preferably Gly)
XIXPNXGGXXXNXXFKX

SEQ ID NO: 152 - protein, 10 aa, Artificial Sequence
  Consensus Light CDR1
  MISC_FEATURE (3)..(3): any amino acid
  MISC_FEATURE (7)..(7): any amino acid
SAXSSVXYMH

SEQ ID NO: 153 - protein, 10 aa, Artificial Sequence
  Consensus Light CDR1
  MISC_FEATURE (3)..(3): Ser or Gly
  MISC_FEATURE (7)..(7): Ser or Asp
SAXSSVXYMH

SEQ ID NO: 154 - protein, 7 aa, Artificial Sequence
  Consensus Light CDR2
  MISC_FEATURE (5)..(5): any amino acid
DTSKXAS

SEQ ID NO: 155 - protein, 7 aa, Artificial Sequence
  Consensus Light CDR2
  MISC_FEATURE (5)..(5): Val or Leu
DTSKXAS

SEQ ID NO: 156 - protein, 7 aa, Artificial Sequence
  Consensus Heavy CDR1
  MISC_FEATURE (4)..(4): any amino acid
  MISC_FEATURE (7)..(7): any amino acid
TFGXGVX

SEQ ID NO: 157 - protein, 7 aa, Artificial Sequence
  Consensus Heavy CDR1
  MISC_FEATURE (4)..(4): Val or Met
  MISC_FEATURE (7)..(7): Ala or Gly
TFGXGVX

SEQ ID NO: 158 - protein, 16 aa, Artificial Sequence
  Consensus Heavy CDR2
  MISC_FEATURE (11)..(11): any amino acid
HIWWDDDKNYXPALKS

SEQ ID NO: 159 - protein, 16 aa, Artificial Sequence
  Consensus Heavy CDR2
  MISC_FEATURE (11)..(11): Asp or Asn
HIWWDDDKNYXPALKS

SEQ ID NO: 160 - protein, 13 aa, Artificial Sequence
  Wild-type peptide
CGAGGVGKSALTI

(19) United States
(12) Patent Application Publication
(10) Pub. No.: US 2023/0109318 A1
(43) Pub. Date: Apr. 6, 2023

(54) KRAS EPITOPES AND ANTIBODIES

(71) Applicant: OBLIQUE THERAPEUTICS AB, Goteborg (SE)

(72) Inventors: Carolina TRKULJA, Hökerum (SE); Max DAVIDSON, Gothenburg (SE); Owe ORWAR, Hovås (SE)

(73) Assignee: OBLIQUE THERAPEUTICS AB, Goteborg (SE)

(21) Appl. No.: 17/801,391

(22) PCT Filed: Feb. 24, 2021
(86) PCT No.: PCT/EP2021/054614

§ 371 Date: Aug. 22, 2022

(30) Foreign Application Priority Data
Feb. 24, 2020 (GB) 2002556.5
Feb. 9, 2021 (GB) 2101780.1

(51) Int. Cl. C07K 16/32; A61P 35/00

(52) U.S. Cl. CPC C07K 16/32; A61P 35/00; A61K 2039/505

(57) ABSTRACT

The present invention relates to antibodies that bind to certain oncogenic mutant forms of KRAS. The invention also relates to certain epitopes of oncogenic mutant forms of KRAS. The invention also relates to immunoconjugates and compositions comprising such antibodies. The invention also provides methods of producing such antibodies. The invention further provides the use of such antibodies for therapeutic purposes, for example in the treatment of cancer.

This invention relates generally to the field of epitopes and antibodies, in particular epitopes of oncogenic KRAS mutant proteins and antibodies that bind to oncogenic KRAS mutant proteins. The invention also relates to therapeutic uses of such antibodies, such as in the treatment of cancer. Antibody-based compositions and methods and uses of the invention also extend to the use of conjugates and other therapeutic combinations, kits and methods.

KRAS (also known as K-Ras or Kirsten rat sarcoma viral oncogene homolog) is a small GTPase (guanosine triphosphatase) that acts as an on/off switch and is a key component in regulation of cell differentiation, proliferation and survival. It is the most frequently mutated oncogene; mutated forms have been found in 22% of all cancers, with mutated oncogenic forms being found in, for example, many colorectal, pancreatic and lung carcinomas. KRAS mutations at residues G12, G13 and Q61 are dominant oncogenic mutations in human cancer. Common oncogenic mutations at residues G12 and G13 of KRAS include G12D, G12V, G12C, G12A, G12S, G12R, G13D and G13C. Substitutions at these residues result in constitutively elevated levels of KRAS-GTP binding due to reduced intrinsic GTP hydrolysis. Although KRAS is considered a holy-grail in cancer research, no direct inhibitors have ever reached the clinic, and KRAS is often described as a target that is “undruggable”. The lack of a suitable binding pocket for a small molecule has been one of the dominant reasons behind this lack of success.

Successful targeting and functional inhibition of oncogenic mutant forms of KRAS could provide a long sought-after solution in the field of cancer therapy.

The present inventors have addressed this need by identifying certain epitopes (or regions) of oncogenic mutant forms of KRAS that have a mutation at position G12 or G13, said epitopes being both accessible to antibodies and particularly useful to target, e.g. with antibodies, in order to bind such oncogenic mutant forms of KRAS and inhibit activity of such oncogenic mutant forms of KRAS. The inventors have identified and generated isolated peptides that correspond to (or correspond essentially to) such epitopes. The inventors have also used such isolated peptides to generate antibodies that bind to and inhibit such oncogenic mutant forms of KRAS.

Thus, in one aspect, the present invention provides an isolated peptide comprising an amino acid sequence selected from the group consisting of (or comprising): SEQ ID NO:17 or a sequence substantially homologous thereto and SEQ ID NO:18 or a sequence substantially homologous thereto.

As discussed elsewhere herein, such isolated peptides may be used as antigenic peptides to generate antibodies that bind to and inhibit activity of oncogenic mutant forms of KRAS that have a mutation at position G12 or G13.

Unless otherwise clear from the context, references to “isolated peptides” or “peptides” of the invention may alternatively be considered references to “isolated epitopes” or “isolated antigenic epitopes”.

In some embodiments, the present invention provides an isolated peptide comprising an amino acid sequence selected from the group consisting of (or comprising): SEQ ID NO:17 and SEQ ID NO:18.

Embodiments in which the “X” residue of SEQ ID NO:17 or SEQ ID NO:18 is a D (aspartic acid) residue are particularly preferred.

Thus, in some embodiments, the present invention provides an isolated peptide comprising an amino acid sequence selected from the group consisting of (or comprising): SEQ ID NO:1 or a sequence substantially homologous thereto and SEQ ID NO:2 or a sequence substantially homologous thereto.

In some embodiments, the present invention provides an isolated peptide comprising an amino acid sequence selected from the group consisting of (or comprising): SEQ ID NO:1 and SEQ ID NO:2.

In some embodiments, the present invention provides an isolated peptide comprising an amino acid sequence of SEQ ID NO:1.

In some embodiments, the present invention provides an isolated peptide comprising an amino acid sequence of SEQ ID NO:2.

In some embodiments, the present invention provides an isolated peptide consisting of an amino acid sequence selected from the group consisting of (or comprising): SEQ ID NO:17 or a sequence substantially homologous thereto and SEQ ID NO:18 or a sequence substantially homologous thereto.

In some embodiments, the present invention provides an isolated peptide consisting of an amino acid sequence selected from the group consisting of (or comprising): SEQ ID NO:17 and SEQ ID NO:18.

In some embodiments in which the isolated peptide comprises (or consists of) an amino acid sequence of SEQ ID NO:17 (or a sequence substantially homologous thereto), preferably the “X” residue is selected from the group consisting of D, V, C, A or R. Thus, embodiments described herein as relating to an amino acid sequence of SEQ ID NO:17 (or a sequence substantially homologous thereto) may, in some preferred embodiments, relate mutatis mutandis to an amino acid sequence of SEQ ID:19 (or a sequence substantially homologous thereto).

In some embodiments in which the isolated peptide comprises an amino acid sequence of SEQ ID NO:17 (or a sequence substantially homologous thereto), preferably the “X” residue is selected from the group consisting of D, C, A, S or R. Thus, embodiments described herein as relating to an amino acid sequence of SEQ ID NO:17 (or a sequence substantially homologous thereto) may, in some preferred embodiments, relate mutatis mutandis to an amino acid sequence of SEQ ID:20 (or a sequence substantially homologous thereto).

In some embodiments in which the isolated peptide comprises an amino acid sequence of SEQ ID NO:17 (or a sequence substantially homologous thereto), preferably the “X” residue is selected from the group consisting of D, C, A or R. Thus, embodiments described herein as relating to an amino acid sequence of SEQ ID NO:17 (or a sequence substantially homologous thereto) may, in some preferred embodiments, relate mutatis mutandis to an amino acid sequence of SEQ ID:21 (or a sequence substantially homologous thereto).

In some embodiments, the present invention provides an isolated peptide consisting of an amino acid sequence selected from the group consisting of (or comprising): SEQ ID NO:1 or a sequence substantially homologous thereto and SEQ ID NO:2 or a sequence substantially homologous thereto.

In some embodiments, the present invention provides an isolated peptide consisting of an amino acid sequence selected from the group consisting of (or comprising): SEQ ID NO:1 and SEQ ID NO:2.

In some embodiments, the present invention provides an isolated peptide consisting of an amino acid sequence of SEQ ID NO:1.

In some embodiments, the present invention provides an isolated peptide consisting of an amino acid sequence of SEQ ID NO:2.

In some embodiments, the isolated peptide may comprise one or more additional amino acids at the N- and/or C-terminus (i.e. in addition to the amino acid sequence of the stated SEQ ID NO). In some preferred embodiments, the isolated peptide may comprise one or more additional amino acids at the N-terminus. In some preferred embodiments, the isolated peptide may comprise one or more additional amino acids at the C-terminus. In some preferred embodiments, the isolated peptide may comprise one or more additional amino acids at the N-terminus and at the C-terminus. In some preferred embodiments, the isolated peptide may comprise a cysteine (C) residue at the N- and/or at the C-terminus (or an additional cysteine (C) residue at the N- and/or at the C-terminus). In some embodiments, the isolated peptide may comprise a cysteine (C) residue at the N-terminus. In some embodiments, the isolated peptide may comprise a cysteine (C) residue at the C-terminus. The provision of a cysteine residue at a terminus of the isolated peptide can permit convenient attachment to a peptide carrier, e.g. as discussed elsewhere herein.

In some embodiments, the isolated peptide may comprise one or more additional modifications at the N- and/or C-terminus. For example, in some embodiments the isolated peptide may be C-terminally amidated. In some embodiments, the isolated peptide may be N-terminally acetylated. Methods of amidating and acetylating peptides are well-known in the art. In some embodiments, the isolated peptide may have a modification (chemical group or linker) that may be used to attach (or link or connect) the peptide to a peptide carrier. A modification (chemical group or linker) that may be used to attach (or link or connect) the peptide to a peptide carrier may be at the N- and/or C-terminus.

In some embodiments, the present invention provides an isolated peptide comprising an amino acid sequence selected from the group consisting of (or comprising): SEQ ID NO:3 or a sequence substantially homologous thereto, SEQ ID NO:4 or a sequence substantially homologous thereto, SEQ ID NO:5 or a sequence substantially homologous thereto and SEQ ID NO:6 or a sequence substantially homologous thereto.

In some embodiments, the present invention provides an isolated peptide comprising an amino acid sequence selected from the group consisting of (or comprising): SEQ ID NO:3 or a sequence substantially homologous thereto and SEQ ID NO:4 or a sequence substantially homologous thereto.

In some embodiments, the present invention provides an isolated peptide comprising an amino acid sequence selected from the group consisting of (or comprising): SEQ ID NO:5 or a sequence substantially homologous thereto and SEQ ID NO:6 or a sequence substantially homologous thereto.

In some embodiments, the present invention provides an isolated peptide comprising an amino acid sequence selected from the group consisting of (or comprising): SEQ ID NO:3 or a sequence substantially homologous thereto and SEQ ID NO:5 or a sequence substantially homologous thereto.

In some embodiments, the present invention provides an isolated peptide comprising an amino acid sequence selected from the group consisting of (or comprising): SEQ ID NO:3, SEQ ID NO:4, SEQ ID NO:5 and SEQ ID NO:6.

The isolated peptide of SEQ ID NO:3 was used to generate the antibody named G12D Ab1 that is described elsewhere herein. The isolated peptide of SEQ ID NO:4 was used to generate the antibody named G12D Ab2 that is described elsewhere herein. The isolated peptide of SEQ ID NO:5 was used to generate the antibody named G13D Ab1 that is described elsewhere herein. The isolated peptide of SEQ ID NO:6 was used to generate the antibody named G13D Ab2 that is described elsewhere herein.

In some embodiments, the present invention provides an isolated peptide comprising an amino acid sequence selected from the group consisting of (or comprising): SEQ ID NO:3 and SEQ ID NO:4.

In some embodiments, the present invention provides an isolated peptide comprising an amino acid sequence selected from the group consisting of (or comprising): SEQ ID NO:5 and SEQ ID NO:6.

In some embodiments, the present invention provides an isolated peptide comprising an amino acid sequence selected from the group consisting of (or comprising): SEQ ID NO:3 and SEQ ID NO:5.

In some embodiments, the present invention provides an isolated peptide comprising the amino acid sequence of SEQ ID NO:3.

In some embodiments, the present invention provides an isolated peptide comprising the amino acid sequence of SEQ ID NO:4.

In some embodiments, the present invention provides an isolated peptide comprising the amino acid sequence of SEQ ID NO:5.

In some embodiments, the present invention provides an isolated peptide comprising the amino acid sequence of SEQ ID NO:6.

In some embodiments, the present invention provides an isolated peptide consisting of an amino acid sequence selected from the group consisting of (or comprising): SEQ ID NO:3 or a sequence substantially homologous thereto, SEQ ID NO:4 or a sequence substantially homologous thereto, SEQ ID NO:5 or a sequence substantially homologous thereto and SEQ ID NO:6 or a sequence substantially homologous thereto.

In some embodiments, the present invention provides an isolated peptide consisting of an amino acid sequence selected from the group consisting of (or comprising): SEQ ID NO:3 or a sequence substantially homologous thereto and SEQ ID NO:4 or a sequence substantially homologous thereto.

In some embodiments, the present invention provides an isolated peptide consisting of an amino acid sequence selected from the group consisting of (or comprising): SEQ ID NO:5 or a sequence substantially homologous thereto and SEQ ID NO:6 or a sequence substantially homologous thereto.

In some embodiments, the present invention provides an isolated peptide consisting of an amino acid sequence selected from the group consisting of (or comprising): SEQ ID NO:3 or a sequence substantially homologous thereto and SEQ ID NO:5 or a sequence substantially homologous thereto.

In some embodiments, the present invention provides an isolated peptide consisting of an amino acid sequence selected from the group consisting of (or comprising): SEQ ID NO:3, SEQ ID NO:4, SEQ ID NO:5 and SEQ ID NO:6.

In some embodiments, the present invention provides an isolated peptide consisting of an amino acid sequence selected from the group consisting of (or comprising): SEQ ID NO:3 and SEQ ID NO:4.

In some embodiments, the present invention provides an isolated peptide consisting of an amino acid sequence selected from the group consisting of (or comprising): SEQ ID NO:5 and SEQ ID NO:6.

In some embodiments, the present invention provides an isolated peptide consisting of an amino acid sequence selected from the group consisting of (or comprising): SEQ ID NO:3 and SEQ ID NO:5.

In some embodiments, the present invention provides an isolated peptide consisting of the amino acid sequence of SEQ ID NO:3.

In some embodiments, the present invention provides an isolated peptide consisting of the amino acid sequence of SEQ ID NO:4.

In some embodiments, the present invention provides an isolated peptide consisting of the amino acid sequence of SEQ ID NO:5.

In some embodiments, the present invention provides an isolated peptide consisting of the amino acid sequence of SEQ ID NO:6.

In the context of the isolated peptide sequences of the invention, a sequence “substantially homologous” to a given amino acid sequence may be a sequence containing 1, 2 or 3 (e.g. 1 or 2) amino acid substitutions or deletions or additions compared to the given amino acid sequence, or a sequence having at least 70% sequence identity to the given amino acid sequence, or a sequence having at least 6 consecutive amino acids of the given amino acid sequence. Other examples of “substantially homologous” sequences are described elsewhere herein in relation to amino acid sequences that are “substantially homologous” to isolated peptides and these examples of “substantially homologous” sequence are also applicable to the specific peptide sequences mentioned above.

In “substantially homologous” isolated peptide sequences in accordance with the present invention the oncogenic mutant (substituted) amino acid residue is not altered. Thus, in isolated peptides comprising (or consisting of) an amino acid sequence that is “substantially homologous” to SEQ ID NO:1, the D residue at (or corresponding to) position 3 of SEQ ID NO:1 is not altered (i.e. it is maintained or is present). In isolated peptides comprising (or consisting of) an amino acid sequence that is “substantially homologous” to SEQ ID NO:2, the D residue at (or corresponding to) position 4 of SEQ ID NO:2 is not altered (i.e. it is maintained or is present). In isolated peptides comprising (or consisting of) an amino acid sequence that is “substantially homologous” to SEQ ID NO:3, the D residue at (or corresponding to) position 3 of SEQ ID NO:3 is not altered (i.e. it is maintained or is present). In isolated peptides comprising (or consisting of) an amino acid sequence that is “substantially homologous” to SEQ ID NO:4, the D residue at (or corresponding to) position 4 of SEQ ID NO:4 is not altered (i.e. it is maintained or is present). In isolated peptides comprising (or consisting of) an amino acid sequence that is “substantially homologous” to SEQ ID NO:5, the D residue at (or corresponding to) position 5 of SEQ ID NO:5 is not altered (i.e. it is maintained or is present). In isolated peptides comprising (or consisting of) an amino acid sequence that is “substantially homologous” to SEQ ID NO:6, the D residue at (or corresponding to) position 4 of SEQ ID NO:6 is not altered (i.e. it is maintained or is present). In isolated peptides comprising (or consisting of) an amino acid sequence that is “substantially homologous” to SEQ ID NO:17, the X residue at (or corresponding to) position 3 of SEQ ID NO:17 is not altered (i.e. it is maintained or is present). In isolated peptides comprising (or consisting of) an amino acid sequence that is “substantially homologous” to SEQ ID NO:18, the X residue at (or corresponding to) position 4 of SEQ ID NO:18 is not altered (i.e. it is maintained or is present). In isolated peptides comprising (or consisting of) an amino acid sequence that is “substantially homologous” to SEQ ID NO:19 or SEQ ID NO:20 or SEQ ID NO:21, the X residue at (or corresponding to) position 3 of SEQ ID NO:19 or SEQ ID NO:20 or SEQ ID NO:21 is not altered (i.e. it is maintained or is present).

In some preferred embodiments, amino acid sequences that are “substantially homologous” to isolated peptides are sequences having, or sequences comprising, a sequence that has, 1, 2, or 3 amino acid substitutions or additions or deletions (preferably 1 or 2, more preferably 1) compared with the amino acid sequence of the given isolated peptide.

Amino acid sequences that are “substantially homologous” to isolated peptides include sequences that comprise (or consist of) at least 5 or at least 6 consecutive amino acids of the isolated peptides (or comprise or consist of at least 7, at least 8, at least 9, at least 10 or at least 11 consecutive amino acids of the isolated peptide). Six amino acids is a typical length of peptide/protein sequence that is recognized or bound by an antibody.

Amino acid sequences that are “substantially homologous” to isolated peptides include sequences having, or sequences comprising (or consisting of) a sequence that has, at least 50%, at least 55%, at least 60%, at least 65%, at least 70%, at least 75%, at least 80%, at least 85%, at least 90%, or at least 95% sequence identity to the given isolated peptide sequence. Sequence identities of at least 70%, at least 75%, at least 80%, at least 85%, at least 90% or at least 95% are preferred.

Alterations in the amino acid sequences can be with conservative or non-conservative amino acids. Preferably said alterations are conservative amino acid substitutions.

A “conservative amino acid substitution”, as used herein, is one in which the amino acid residue is replaced with another amino acid residue having a similar side chain. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine, cysteine), nonpolar side chains (e.g. glycine, cysteine, alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine).

The term “substantially homologous” also includes modifications or chemical equivalents of the amino acid sequences of the present invention that perform substantially the same function as the proteins of the invention in substantially the same way. For example, any substantially homologous isolated peptide should typically retain the ability to act as peptide or epitope to (or against) which anti-antibodies which bind to an oncogenic mutant form of KRAS in accordance with the invention can be generated (or raised).

Methods of carrying out the above described manipulation of amino acids (e.g. to generate “substantially homologous” sequences) are well known to a person skilled in the art.

In some embodiments, the isolated peptides do not contain any internal cysteine residues. By “internal” residue is meant a residue at a position other than the N-terminal and/or C-terminal residue. Thus, in some embodiments, a sequence that is “substantially homologous” to a given amino acid sequence does not have a cysteine (C) residue as the substituting or additional amino acid.

Homology (e.g. sequence identity) may be assessed by any convenient method. However, for determining the degree of homology (e.g. identity) between sequences, computer programs that make multiple alignments of sequences are useful, for instance Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) and those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:11-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al., Nucleic Acids Res., 25:3389-3402, 1997), BLASTP, BLASTN, or GCG (Devereux, Haeberli, Smithies, Nucleic Acids Res., 12:387, 1984) are also useful for this purpose. Furthermore, the Dali server at the European Bioinformatics institute offers structure-based alignments of protein sequences (Holm, Trends in Biochemical Sciences, 20:478-480, 1995; Holm, J. Mol. Biol., 233:123-38, 1993; Holm, Nucleic Acid Res., 26:316-9, 1998).

By way of providing a reference point, sequences according to the present invention having 65%, 70%, 75%, 80%, 85%, 90%, 95%, 96%, 97%, 98% or 99% homology, sequence identity etc. may be determined using the ALIGN program with default parameters (for instance available on Internet at the GENESTREAM network server, IGH, Montpellier, France).

In some embodiments, the present invention provides an isolated peptide that comprises (or consists of) an elongated or truncated or cyclic version of an isolated peptide sequence disclosed herein (or a sequence substantially homologous thereto). Elongated, truncated and cyclic versions of peptides are discussed elsewhere herein.

An isolated peptide of the invention may comprise (or consist of) an elongated version of an isolated peptide sequence disclosed herein, or an elongated version of an amino acid sequence substantially homologous to an isolated peptide sequence disclosed herein. For example, one or more additional amino acids (e.g. at least 2, at least 3, at least 4, at least 5, at least 6, at least 7, at least 8 or at 9, at least 10, at least 15 or at least 20 amino acids, or 1-5 or 1-10 or 1-amino acids) may be present at one end or both ends of the isolated peptide sequence (or sequence substantially homologous thereto).

An isolated peptide of the invention may comprise (or consist of) a truncated version of an isolated peptide sequence disclosed herein, or a truncated version of an isolated peptide sequence disclosed herein. For example, one or more amino acids (e.g. at least 2 or at least 3, e.g. 1-3 or 1-2 or 1) may be absent from one end or both ends of the isolated peptide sequence (or sequence substantially homologous thereto).

In some embodiments, isolated peptides may be at least 5, at least 6, at least 7, at least 8, at least 9, at least 10 amino acids, at least 11 amino acids or at least 12 amino acids in length, for example 6 to 10, 6 to 12, 6 to 15, 6 to 20, 6 to 25, 6 to 30, 6 to 40, 6 to 50, 6 to 60, or 6 to 75 amino acids in length. Isolated peptides may be, for example, 5 to 7, 5 to 8, 5 to 9, 5 to 10, 5 to 11, 5 to 12, 5 to 13, 5 to 14, to 15, 5 to 20, 5 to 25, 5 to 30, 5 to 40, 5 to 50, 5 to 60, 5 to 70, 5 to 75 amino acids in length. Isolated peptides may be, for example, 12 to 15, 12 to 20, 12 to 25, 12 to 30, 12 to 40, 12 to 50, 12 to 60, 12 to 70, 12 to 75 amino acids in length.

In some embodiments, isolated peptides may be ≤50 amino acids in length, e.g. ≤45, ≤40, ≤35, ≤30, ≤25, ≤20, ≤15 or ≤10 amino acids in length (e.g. 5-10, 5-15, 5-20, 5-25, 5-30, 5-35, 5-40, 5-45, 5-50, 6-10, 6-15, 6-20, 6-25, 6-30, 6-35, 6-40, 6-45, 6-50, 8-10, 8-15, 8-20, 8-25, 8-30, 8-35, 8-40, 8-45, 8-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 25-30, 25-35 or 25-40, 25-45, 25-50, 30-35, 30-40, 30-45, 30-50, 35-40, 35-45, 35-50, 40-45, 40-50 or 45-50 amino acids in length).

In some embodiments, isolated peptides may be ≤20 amino acids in length, e.g. ≤15, ≤14, ≤13, ≤12, ≤11, ≤10 amino acids in length (e.g. 5-10, 5-15, 5-20, 6-10, 6-15, 6-20, 8-10, 8-15, 8-20, 10-15, 10-20 or 15-20 amino acids in length). In some embodiments, isolated peptides may be 5, 6, 7, 8, 9, 10, 11, 12, 13, 14, 15, 16, 17, 18, 19 or 20 amino acids in length.). In some embodiments, isolated peptides may be 5, 6, 7, 8, 9, 10, 11, 12, 13, 14, 15, 16, 17, 18, 19 or 20 amino acids in length.

In some embodiments, isolated peptides may be 12 amino acids in length. In some embodiments, isolated peptides may be 13 amino acids in length.

Typically and preferably, the isolated peptides are linear peptides (or linear epitopes).

Methods for synthesising peptides are well known in the art. A common technique used for preparing linear peptides (e.g. to be used for immunization and antibody generation) is Fmoc SPPS (Solid Phase Peptide Synthesis). In SPPS, small porous beads are treated with functional linkers on which peptide chains can be built using repeated cycles of wash-coupling-wash. The synthesized peptide is then released from the beads using chemical cleavage. For synthesis of cyclic peptides, common methods utilize cyclization by formation of a disulphide bridge (where the bridge is formed bridge by two cysteines of the peptide, e.g. one at the N-terminus and one at the C-terminus), or by formation of a “head-to-tail” bridge where the bridge consists of a typical peptide bond. Cyclic peptides can be formed on a solid support.

Other methods for synthesising peptides include using other chemical synthesis procedures, in vitro translation, or by introducing a suitable expression vector into cells.

Isolated peptides in accordance with the present invention of course do not include a full-length oncogenic mutant form of KRAS in accordance with the invention, or any other full-length (e.g. wild-type or native) protein in the KRAS family, or any other full-length (wild-type) proteins. Isolated peptides in accordance with the present invention thus do not include full-length SEQ ID NO:7, 8, 9, 10, 11, 12, 13, 14, 15 or 16.

Isolated peptides of the present invention, although corresponding to (or corresponding essentially to) regions (or epitopes) of a full-length oncogenic mutant form of KRAS in accordance with the invention (e.g. as described elsewhere herein), do not themselves occur in nature (i.e. they do not have naturally occurring counterparts or do not occur in isolation in nature). Thus, the isolated peptides of the invention can be considered to be artificial peptides, or synthetic peptides, or man-made peptides, or non-native peptides.

A further aspect of the invention provides a conjugate. Typically, the conjugate is configured to be used for the production of antibodies. The conjugate may comprise at least one isolated peptide as defined above coupled to (i.e. linked to or connected to or bonded to), or admixed with, a peptide carrier.

Thus, in one aspect, the invention provides a conjugate comprising an isolated peptide of the invention. Conjugates typically comprise an isolated peptide of the invention and a peptide carrier, wherein said isolated peptide is coupled to, or admixed with, said peptide carrier. Peptide carriers typically enhance immunogenicity. This may be useful as, in some cases, short peptides which provide (or represent or correspond to) an antigenic epitope are, by themselves, too small to induce an immune response.

Peptide carriers are typically large macromolecules such as proteins, polysaccharides or polymeric amino acids. In some embodiments, the peptide carrier is selected from the group consisting of keyhole limpet hemocyanin (KLH), ovalbumin (OVA), serum albumins, polylysine and the like. KLH is typically preferred.

The coupling of an isolated peptide of the invention to a peptide carrier can, for example, be a covalent coupling or a disulphide bridge. In some embodiments, an isolated peptide of the invention may be provided with an (additional) cysteine residue at its N- or C-terminus (e.g. as described elsewhere herein). Such a cysteine residue typically facilitates coupling of the isolated peptide to a peptide carrier (e.g. KLH). In some embodiments, the isolated peptide may have a modification (e.g. a chemical group such as a propargyl group) that permits coupling of the isolated peptide to a peptide carrier. In some embodiments, the isolated peptide is coupled to a peptide carrier via standard cross-linking agent (e.g. glutaraldehyde). Methods of linking isolated peptides to peptide carriers are well known in the art.

In some embodiments, isolated peptides (or conjugates) in accordance with the invention may be present in a solution or in a suspension. Thus, in one aspect the present invention provides a composition comprising an isolated peptide of the invention, and optionally an acceptable (e.g. a pharmaceutically acceptable) diluent, buffer, preservative and/or excipient.

In some embodiments, isolated peptides (or conjugates) may be present on (i.e. attached to or bound to) a solid support (e.g. a bead or microbead or plate or microtitre plate). Thus, in one aspect the present invention provides a solid support, having attached thereto (either directly or indirectly attached thereto) an isolated peptide or conjugate of the invention.

Isolated peptides (and conjugates) of the invention are typically suitable for use in the identification (or generation or raising) of antibodies that bind to an oncogenic mutant form of KRAS in accordance with the invention. For example, isolated peptides of the present invention are typically suitable for use as antigenic epitopes for the identification (or the generation or the raising) of antibodies. The identification (or the generation or the raising) of antibodies using isolated peptides of the invention may be done by any suitable means and the skilled person is familiar with suitable techniques (e.g. as discussed elsewhere herein). For example, isolated peptides (and conjugates) of the invention are typically suitable for use in the identification (or generation or raising) of polyclonal antibodies that bind to an oncogenic mutant form of KRAS in accordance with the invention (e.g. polyclonal antibodies raised in an animal such as a rabbit that has been immunized with an isolated peptide (or conjugate) of the invention), or in in the identification (or generation or raising) of monoclonal antibodies using standard hybridoma technology or phage display. Put another way, isolated peptides (and conjugates) of the invention typically represent (or correspond to or correspond essentially to) useful epitopes of oncogenic mutant forms of KRAS in accordance with the invention to target with anti-KRAS antibodies in accordance with the invention.

Typically, isolated peptides (and conjugates) of the invention are suitable for use in the identification (or generation or raising) of antibodies that bind to and inhibit activity of an oncogenic mutant form of KRAS in accordance with the invention.

Isolated peptides (and conjugates) of the invention correspond to (or correspond essentially to) an epitope (or region or portion) of an oncogenic mutant form of KRAS in accordance with the invention that is positioned (or located) in the region of such an oncogenic mutant form of KRAS (e.g. SEQ ID NO: 8, 9, 11, 12, 13, 14, 15 or 16) from amino acid residue 10 to amino acid residue 21.

Nucleic acid molecules comprising (or consisting of) nucleotide sequences that encode the isolated peptides of the present invention as defined herein, or nucleic acid molecules substantially homologous thereto, form yet further aspects of the invention.

The term “substantially homologous” as used herein in connection with an nucleic acid sequence includes sequences having at least 65%, 70% or 75%, preferably at least 80%, and even more preferably at least 85%, 90%, 95%, 96%, 97%, 98% or 99%, sequence identity to the starting nucleic acid sequence.

The term “nucleic acid sequence” or “nucleic acid molecule” as used herein refers to a sequence of nucleoside or nucleotide monomers composed of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases.

Examples of such modified bases include aza and deaza adenine, guanine, cytosine, thymidine and uracil; and xanthine and hypoxanthine. The nucleic acid molecules may be double stranded or single stranded. The nucleic acid molecules may be wholly or partially synthetic or recombinant.

In another aspect, the present invention provides a composition comprising an isolated peptide (or conjugate) of the invention. Such compositions may further comprise (e.g. be in admixture with) a suitable diluent, carrier, excipient and/or preservative (e.g. a pharmaceutically acceptable diluent, carrier, excipient and/or preservative).

As indicated above, isolated peptides (and conjugates) of the invention are typically suitable for use in the identification (or generation or raising) of antibodies that bind to that bind to and inhibit activity of an oncogenic mutant form of KRAS in accordance with the invention.

Thus, in one aspect, the present invention provides an antibody, for example an isolated antibody, which binds to (or specifically recognises or specifically binds to) an oncogenic mutant form of KRAS, said oncogenic mutant form of KRAS comprising an amino acid substitution at the position corresponding to position G12 or G13 of wild-type KRAS, wherein said antibody binds to an epitope that is in the region of said oncogenic mutant form of KRAS that is defined by the amino acid residues that correspond to amino acid residues 10-21 of wild-type KRAS, and wherein said antibody inhibits activity of said oncogenic mutant form of KRAS.

In preferred embodiments, the oncogenic mutant form of KRAS (or K-Ras) is a human oncogenic mutant form of KRAS (or K-Ras) (i.e. is a human protein).

There are two isoforms of human KRAS. These are KRAS isoform 2A and KRAS isoform 2B. The wild-type KRAS referred to in relation to the present invention may be KRAS isoform 2A or KRAS isoform 2B. The amino acid sequence of wild-type KRAS isoform 2A is set forth herein as SEQ ID NO:7. The amino acid sequence of wild-type KRAS isoform 2B is set forth herein as SEQ ID NO:10. Thus, in some embodiments, the wild-type KRAS referred to in relation to the present invention has the amino acid sequence of SEQ ID NO:7. In some embodiments, the wild-type KRAS referred to in relation to the present invention has the amino acid sequence of SEQ ID NO:10. Wild-type KRAS isoform 2A and wild-type KRAS isoform 2B have identical sequences at amino residues 10-21 (indeed SEQ ID NO:7 and SEQ ID NO:10 are identical over residues 1 to 150).

An oncogenic mutant form of KRAS in accordance with the invention may be an oncogenic mutant form of KRAS isoform 2A or an oncogenic mutant form of KRAS isoform 2B in accordance with the invention.

Thus, in some embodiments, an oncogenic mutant form of KRAS has the amino acid sequence of SEQ ID NO:13, 14, 15 or 16.

In some embodiments, an oncogenic mutant form of KRAS has the amino acid sequence of SEQ ID NO:13 or SEQ ID NO:14.

In some embodiments, an oncogenic mutant form of KRAS has the amino acid sequence of SEQ ID NO:15 or SEQ ID NO:16.

In some embodiments, an oncogenic mutant form of KRAS has the amino acid sequence of SEQ ID NO:13 or SEQ ID NO:15.

In some embodiments, an oncogenic mutant form of KRAS has the amino acid sequence of SEQ ID NO:14 or SEQ ID NO:16.

In some embodiments in which an oncogenic mutant form of KRAS has the amino acid sequence of SEQ ID NO:13 or SEQ ID NO:15, preferably the “X” residue is selected from the group consisting of D, V, C, A or R. Thus, embodiments described herein as relating to an amino acid sequence of SEQ ID NO:13 may, in some preferred embodiments, relate mutatis mutandis to an amino acid sequence of SEQ ID:22. Embodiments described herein as relating to an amino acid sequence of SEQ ID NO:15 may, in some preferred embodiments, relate mutatis mutandis to an amino acid sequence of SEQ ID:23.

In some embodiments in which an oncogenic mutant form of KRAS has the amino acid sequence of SEQ ID NO:13 or SEQ ID NO:15, preferably the “X” residue is selected from the group consisting of D, C, A, S or R. Thus, embodiments described herein as relating to an amino acid sequence of SEQ ID NO:13 may, in some preferred embodiments, relate mutatis mutandis to an amino acid sequence of SEQ ID:24. Embodiments described herein as relating to an amino acid sequence of SEQ ID NO:15 may, in some preferred embodiments, relate mutatis mutandis to an amino acid sequence of SEQ ID:25.

In some embodiments in which an oncogenic mutant form of KRAS has the amino acid sequence of SEQ ID NO:13 or SEQ ID NO:15, preferably the “X” residue is selected from the group consisting of D, C, A or R. Thus, embodiments described herein as relating to an amino acid sequence of SEQ ID NO:13 may, in some preferred embodiments, relate mutatis mutandis to an amino acid sequence of SEQ ID:26. Embodiments described herein as relating to an amino acid sequence of SEQ ID NO:15 may, in some preferred embodiments, relate mutatis mutandis to an amino acid sequence of SEQ ID:27.

In preferred embodiments, the X residue in SEQ ID NO:13, SEQ ID NO:14, SEQ ID NO:15 or SEQ ID NO:16 is a D residue.

Thus, in preferred embodiments, the amino acid substitution (or mutant residue) in the oncogenic mutant form of KRAS at the position corresponding to position G12 of wild-type KRAS is a G12D substitution (i.e. D is preferably the mutant residue). In preferred embodiments, the amino acid substitution (or mutant residue) in the oncogenic mutant form of KRAS at the position corresponding to position G13 of wild-type KRAS is a G13D mutation (i.e. D is preferably the mutant residue).

In some preferred embodiments, an oncogenic mutant form of KRAS has the amino acid sequence of SEQ ID NO:8, 9, 11 or 12.

In some embodiments, an oncogenic mutant form of KRAS has the amino acid sequence of SEQ ID NO:8 or SEQ ID NO:9.

In some embodiments, an oncogenic mutant form of KRAS has the amino acid sequence of SEQ ID NO:11 or SEQ ID NO:12.

In some embodiments, an oncogenic mutant form of KRAS has the amino acid sequence of SEQ ID NO:8 or SEQ ID NO:11.

In some embodiments, an oncogenic mutant form of KRAS has the amino acid sequence of SEQ ID NO:9 or SEQ ID NO:12.

In some embodiments, an oncogenic mutant form of KRAS has the amino acid sequence of SEQ ID NO:8 (isoform 2A, G12D mutation).

In some embodiments, an oncogenic mutant form of KRAS has the amino acid sequence of SEQ ID NO:9 (isoform 2A, G13D mutation).

In some embodiments, an oncogenic mutant form of KRAS has the amino acid sequence of SEQ ID NO:11 (isoform 2B, G12D mutation).

In some embodiments, an oncogenic mutant form of KRAS has the amino acid sequence of SEQ ID NO:12 (isoform 2B, G13D mutation).

The G12D oncogenic mutant forms of KRAS isoform 2A (SEQ ID NO:8) and KRAS isoform 2B (SEQ ID NO:11) have identical sequences at amino residues 10-21. The G13D oncogenic mutant forms of KRAS isoform 2A (SEQ ID NO:9) and KRAS isoform 2B (SEQ ID NO:12) have identical sequences at amino residues 10-21.

In some embodiments, an antibody of the invention may bind to an oncogenic mutant form of KRAS comprising an amino acid substitution at the position corresponding to position G12 of wild-type KRAS (e.g. SEQ ID NO:8, 11, 13 or 15).

In some embodiments, an antibody of the invention may bind to an oncogenic mutant form of KRAS comprising an amino acid substitution at the position corresponding to position G13 of wild-type KRAS (e.g. SEQ ID NO:9, 12, 14 or 16).

In some embodiments, an antibody of the invention may bind to an oncogenic mutant form of KRAS comprising an amino acid substitution at the position corresponding to position G12 of wild-type KRAS (e.g. SEQ ID NO:8, 11, 13 or 15) and bind to an oncogenic mutant form of KRAS comprising an amino acid substitution at the position corresponding to position G13 of wild-type KRAS (e.g. SEQ ID NO:9, 12, 14 or 16).

Amino acids 10-21 of each of the oncogenic mutant forms of KRAS set out in SEQ ID NOs: 8, 9, 11, 12, 13, 14, 15 and 16 correspond to amino acid region 10-21 of the corresponding wild-type sequence (SEQ ID NO:7 or SEQ ID NO:10), with the exception of course of the relevant oncogenic mutant amino acid residue (amino acid substitution) at position 12 or position 13.

Thus, in embodiments in which the oncogenic mutant form of KRAS has an amino acid sequence of SEQ ID NO: 8, 9, 11, 12, 13, 14, 15 or 16, an antibody of the invention binds to an epitope of one of more of said oncogenic mutant forms of KRAS in the region defined by amino acid residues 10-21 of one or more of said sequences.

Preferred oncogenic mutant forms of KRAS are also discussed elsewhere herein.

As discussed elsewhere herein, antibodies of the present invention typically bind to an epitope of an oncogenic mutant form of KRAS in accordance with the invention that is in the region of said oncogenic mutant form of KRAS that is defined by the amino acid residues that correspond to amino acid residues 10-21 of wild-type KRAS. In some embodiments, the entire epitope bound lies within this region of an oncogenic mutant form of KRAS in accordance with the invention. In some embodiments, at least one amino acid of the epitope bound lies within this region of an oncogenic mutant form of KRAS in accordance with the invention.

In preferred embodiments, antibodies of the invention bind to (or are capable of binding to) an isolated peptide or conjugate of the invention. Preferred isolated peptides and conjugates and preferred groups of isolated peptides and conjugates are defined elsewhere herein. In preferred embodiments, antibodies of the invention bind to a preferred isolated peptide or conjugate as described elsewhere herein. The ability of an antibody to bind to an isolated peptide can be assessed by any appropriate means and skilled person is familiar with suitable methods (e.g. an ELISA assay such as an ELISA assay to assess whether or not a given isolated peptide can compete with a full length oncogenic mutant form of KRAS in accordance with the invention for antibody binding).

A suitable ELISA assay for assessing the ability of an antibody to bind to an isolated peptide (or conjugate) is described in the Example section herein.

In some embodiments, antibodies of the invention bind to (or are capable of binding to) an isolated peptide that comprises (or consists of) an amino acid sequence selected from the group consisting of (or comprising) SEQ ID NO:17, SEQ ID NO:18, SEQ ID NO:1 and SEQ ID NO:2, or bind to (or are capable of binding to) a conjugate comprising such an isolated peptide.

In some embodiments, antibodies of the invention bind to (or are capable of binding to) an isolated peptide that comprises (or consists of) an amino acid sequence selected from the group consisting of (or comprising) SEQ ID NO:1 and SEQ ID NO:2, or bind to (or are capable of binding to) a conjugate comprising such an isolated peptide.

In some embodiments, antibodies of the invention bind to (or are capable of binding to) an isolated peptide that comprises (or consists of) an amino acid sequence selected from the group consisting of (or comprising) SEQ ID NO:3, SEQ ID NO:4, SEQ ID NO:5 and SEQ ID NO:6, or bind to (or are capable of binding to) a conjugate comprising such an isolated peptide.

In some embodiments, an antibody of the invention may bind to at least one isolated peptide of the invention that comprises an amino acid sequence of SEQ ID NO:17 and at least one isolated peptide of the invention that comprises an amino acid sequence of SEQ ID NO:18.

In some embodiments, an antibody of the invention may bind to at least one isolated peptide of the invention that comprises an amino acid sequence of SEQ ID NO:1 and at least one isolated peptide of the invention that comprises an amino acid sequence of SEQ ID NO:2.

In some embodiments, an antibody of the invention may bind to at least one isolated peptide of the invention that consists of an amino acid sequence of SEQ ID NO:3 or SEQ ID NO:4 (preferably SEQ ID NO:3) and at least one isolated peptide of the invention that comprises an amino acid sequence of SEQ ID NO:5 or SEQ ID NO:6 (preferably SEQ ID NO:5).

Although preferred antibodies of the invention bind to (or are capable of binding to) an isolated peptide or conjugate of the invention, they of course also typically bind to full-length (or native) oncogenic mutant forms of KRAS in accordance with the invention (preferably human oncogenic mutant forms of KRAS in accordance with the invention, e.g. as set forth in SEQ ID NOs 8, 9, 11, 12, 13, 14, 15 or 16, preferably 8, 9, 11 or 12).

In some embodiments, a full-length (or native) oncogenic mutant form of KRAS in accordance with the invention (preferably human oncogenic mutant form of KRAS in accordance with the invention) is an oncogenic mutant form of KRAS that is expressed in cells, preferably in mammalian cells, more preferably in human cells. Thus, in preferred embodiments, an oncogenic mutant form of KRAS in accordance with the invention is an oncogenic mutant form of KRAS in cells (or expressed in cells). In preferred embodiments, said cells are cancer cells or a cancer cell line. Such cancer cells or cell lines may include, but are not limited to, breast cancer cells or a breast cancer cell line (e.g. the cell line MDA-MB-231), lung cancer cells or a lung cancer cell line (e.g. the cell line SK-LU1), colon cancer cells or a colon cancer cell line (e.g. the cell line HCT116 or the cell line LS174T), or pancreatic cancer cells or a pancreatic cancer cell line (e.g. MIA-PA-CA-2). The breast cancer cell line MDA-MB-231 is a human cell line that carries a G13D mutation in KRAS. The lung cancer cell line SK-LU1 is a human cell line that carries a G12D mutation in KRAS. The colon cancer cell line HCT116 is a human cell line that carries a G13D mutation in KRAS. The colon cancer cell line LS174T is a human cell line that carries a G12D mutation in KRAS. The colon cancer cell line SW620 is a human colorectal adenocarcinoma cell line that carries a G13V mutation in KRAS. The pancreatic cancer cell line MIA-PA-CA-2 is a human cell line that carries a G12C mutation in KRAS. The binding of an antibody of the invention to an oncogenic mutant form of KRAS in accordance with the invention may be assessed by any suitable means, and the skilled person will be familiar with suitable methods (e.g. ELISA assay or immunocytochemistry or using a functional assay e.g. as described elsewhere herein).

The isolated peptides of the present invention correspond to, or correspond essentially to, certain regions or epitopes of full-length oncogenic mutant forms of KRAS in accordance with the invention (preferably human oncogenic mutant forms of KRAS in accordance with the invention, e.g. as set forth in SEQ ID NOs 8, 9, 11, 12, 13, 14, 15 or 16).

The isolated peptide of SEQ ID NO:17 corresponds to residues 10-21 of a human oncogenic mutant form of KRAS in accordance with the invention as set forth in SEQ ID NO:13 or SEQ ID NO:15.

The isolated peptide of SEQ ID NO:18 corresponds to residues 10-21 of a human oncogenic mutant form of KRAS in accordance with the invention as set forth in SEQ ID NO:14 or SEQ ID NO:16.

The isolated peptide of SEQ ID NO:1 corresponds to residues 10-21 of a human oncogenic mutant form of KRAS in accordance with the invention as set forth in SEQ ID NO:8 or SEQ ID NO:11.

The isolated peptide of SEQ ID NO:2 corresponds to residues 10-21 of a human oncogenic mutant form of KRAS in accordance with the invention as set forth in SEQ ID NO:9 or SEQ ID NO:12.

The isolated peptides of SEQ ID NO:3 and SEQ ID NO:4 correspond essentially to residues 10-21 of a human oncogenic mutant form of KRAS in accordance with the invention as set forth in SEQ ID NO:8 or SEQ ID NO:11.

The isolated peptides of SEQ ID NO:5 and SEQ ID NO:6 correspond essentially to residues 10-21 of a human oncogenic mutant form of KRAS in accordance with the invention as set forth in SEQ ID NO:9 or SEQ ID NO:12.

By “corresponds to” in the context of isolated peptide sequences is meant that the amino sequence (SEQ ID NO:) of the isolated peptide matches the amino acid sequence of the stated region or epitope of a human oncogenic mutant form of KRAS in accordance with the invention. By “corresponds essentially to” is meant that the amino acid sequence of the isolated peptide (SEQ ID NO:) is identifiable as being based on (or derived from or a modified form of) the sequence of the stated region or epitope of a human oncogenic mutant form of KRAS in accordance with the invention. For example, an isolated peptide having a sequence that “corresponds essentially to” the stated region or epitope of a human oncogenic mutant form of KRAS in accordance with the invention typically has one or more (e.g. 1, 2 or 3) amino acid substitutions, additions or deletions as compared to an isolated peptide that corresponds to (i.e. exactly corresponds to) the sequence of the stated region or epitope of a human oncogenic mutant form of KRAS in accordance with the invention. Thus, an isolated peptide having a sequence that “corresponds essentially to” the stated region or epitope of a human oncogenic mutant form of KRAS in accordance with the invention may be considered to be a “substantially homologous” isolated peptide sequence as defined elsewhere herein.

As indicated above, antibodies of the present invention bind to at least one oncogenic mutant form of KRAS in accordance with the present invention.

In some embodiments, antibodies of the present invention bind to a oncogenic mutant form of KRAS having an amino acid sequence of SEQ ID NO:13, 14, 15 or 16. In some embodiments, antibodies of the present invention bind to at least one oncogenic mutant form of KRAS having an amino acid sequence of SEQ ID NO:13 or 15 and to at least one oncogenic mutant form of KRAS having an amino acid sequence of SEQ ID NO:14 or 16.

In some embodiments, antibodies of the present invention bind to a oncogenic mutant form of KRAS having an amino acid sequence of SEQ ID NO:8, 9, 11 or 12. In some embodiments, antibodies of the present invention bind to at least one oncogenic mutant form of KRAS having an amino acid sequence of SEQ ID NO:8 or 11 and to at least one oncogenic mutant form of KRAS having an amino acid sequence of SEQ ID NO:9 or 12.

In some embodiments, antibodies of the present invention bind to (and preferably inhibit activity of) an oncogenic mutant form of KRAS which has a given oncogenic mutant residue at position 12 (e.g. one of the potential position 12 residues as set forth in SEQ ID NO:13 or SEQ ID NO:15, i.e. D or V or C or A or S or R) and also bind (i.e. additionally bind or are additionally capable of binding) to an oncogenic mutant form of KRAS which has said given (i.e. has the same) oncogenic mutant residue at position 13 instead of at position 12.

For example, in some embodiments, antibodies of the present invention bind to (and preferably inhibit activity of) an oncogenic mutant form of KRAS which has a D (aspartic acid) residue at position 12 and also bind (i.e. additionally bind or are additionally capable of binding) to an oncogenic mutant form of KRAS which has a D (aspartic acid) residue at position 13 instead of at position 12.

In some embodiments, antibodies of the present invention bind to (and preferably inhibit activity of) an oncogenic mutant form of KRAS which has a given oncogenic mutant residue at position 13 (e.g. one of the potential position 13 residues as set forth in SEQ ID NO:14 or SEQ ID NO:16, i.e. D or C) and also bind (i.e. additionally bind or are additionally capable of binding) to an oncogenic mutant form of KRAS which has said given (i.e. has the same) oncogenic mutant residue at position 12 instead of at position 13.

Without wishing to be bound by theory, it is believed that, in some cases at least, the nature (i.e. identity) of the oncogenic mutant residue may be as (or more) important than its precise position (i.e. position 12 versus 13) given the small difference in distance between positions 12 and 13. Thus, in some embodiments, antibodies that bind (and preferably inhibit) an oncogenic mutant form of KRAS having a given mutant residue at position 12 may also bind to one or more other oncogenic mutant forms of KRAS having the same given mutant residue at position 13 instead of at position 12. Likewise, in some embodiments, antibodies that bind (and preferably inhibit) an oncogenic mutant form of KRAS having a given mutant residue at position 13 may also bind to one or more other oncogenic mutant forms of KRAS having the same given mutant residue at position 12 instead of at position 13. In the case of G12D and G13D mutant KRAS, it is believed that certain antibodies of the invention bind to the negatively charged aspartic acid (D) residue, but that certain antibodies can accommodate the D residue at either position 12 or 13 due to the small difference in distance.

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:148 or preferably SEQ ID NO:149, or a sequence substantially homologous thereto,

(b) a VH CDR2 that has the amino acid sequence of SEQ ID NO:150 or preferably SEQ ID NO:151, or a sequence substantially homologous thereto, and

(c) a VH CDR3 that has the amino acid sequence of SEQ ID NO:34 or 54 or 74, or a sequence substantially homologous thereto; and/or (preferably “and”) wherein said light chain variable region comprises:

(d) a variable light (VL) CDR1 that has the amino acid sequence of SEQ ID NO:152 or preferably SEQ ID NO:153, or a sequence substantially homologous thereto,

(e) a VL CDR2 that has the amino acid sequence of SEQ ID NO:154 or preferably 155, or a sequence substantially homologous thereto, and

(f) a VL CDR3 that has the amino acid sequence of SEQ ID NO:37, SEQ ID NO:57 or SEQ ID NO:77, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments of the present invention, the VH CDR1 has or comprises an amino acid sequence of SEQ ID NO: 148 (X₁ Y X₃ M H). In these embodiments X₁ or X₃ can be any amino acid. Preferably the X₁ residue is D or R. Thus, a preferred VH CDR1 has or comprises the amino acid sequence of SEQ ID NO:149. For example, preferred VH CDR1 sequences of this embodiment have or comprise SEQ ID NOs: 32, 52 or 72.

In some embodiments of the present invention, the VH CDR2 has or comprises an amino acid sequence of SEQ ID NO: 150 (X₁ I X₃ P N X₆ G G X₉ X₁₀ X₁₁ N X₁₃X₁₄F K X₁₇). In these embodiments, X₁, X₃, X₆, X₉, X₁₀, X₁₁, X₁₃, X₁₄ and X₁₇ can be any amino acid. Preferably one or more, most preferably all, of these X residues are selected from the following group: X₁ is Y or R (preferably Y); X₃ is N or D (preferably N); X₆ is N or S (preferably N); X₉ is A or T; X₁₀ is S or Y or T; X₁₁ is Y or F (preferably Y); X₁₃ is Q or E (preferably Q), X₁₄ is K or R; X₁₇ is G or S (preferably G). Thus, a preferred VH CDR2 has or comprises the amino acid sequence of SEQ ID NO:151. For example, preferred VH CDR2 sequences of this embodiment have or comprise SEQ ID NOs: 33, 53 or 73.

In some embodiments of the present invention, the VL CDR1 has or comprises an amino acid sequence of SEQ ID NO: 152 (S A X₃ S S V X₇ Y M H). In these embodiments X₃ and X₇ can be any amino acid. Preferably one or both of these X residues are selected from the following group: X₃ is S or G; X₇ is S or D. Thus, a preferred VL CDR1 has or comprises the amino acid sequence of SEQ ID NO: 153. For example, preferred VL CDR1 sequences of this embodiment have or comprise SEQ ID NOs: 35, 55 or 75.

In some embodiments of the present invention, the VL CDR2 has or comprises an amino acid sequence of SEQ ID NO: 154 (D T S K X₅ A S). In these embodiments X₅ can be any amino acid. Preferably, X₅ is V or L. Thus, a preferred VL CDR1 has or comprises the amino acid sequence of SEQ ID NO:155. For example, preferred VL CDR1 sequences of this embodiment have or comprise SEQ ID NOs: 36, 56 or 76.

Certain preferred combinations of VH CDR sequences and VL CDR sequences are set forth in each of the rows numbered 1-15 in Table H below:

TABLE H

| | VH | VH | VH | VL | VL | VL |
|---|---|---|---|---|---|---|
| | CDR1 | CDR2 | CDR3 | CDR1 | CDR2 | CDR3 |
| | (SEQ | (SEQ | (SEQ | (SEQ | (SEQ | (SEQ |
| | ID NO:) | ID NO:) | ID NO:) | ID NO:) | ID NO:) | ID NO:) |
| 1 | 148 | 33 | 34 | 35 | 36 | 37 |
| 2 | 148 | 53 | 54 | 55 | 56 | 57 |
| 3 | 148 | 73 | 74 | 75 | 76 | 77 |
| 4 | 32 | 150 | 34 | 35 | 36 | 37 |
| 5 | 52 | 150 | 54 | 55 | 56 | 57 |
| 6 | 72 | 150 | 74 | 75 | 76 | 77 |
| 7 | 32 | 33 | 34 | 152 | 36 | 37 |
| 8 | 52 | 53 | 54 | 152 | 56 | 57 |
| 9 | 72 | 73 | 74 | 152 | 76 | 77 |
| 10 | 32 | 33 | 34 | 35 | 154 | 37 |
| 11 | 52 | 53 | 54 | 55 | 154 | 57 |
| 12 | 72 | 73 | 74 | 75 | 154 | 77 |
| 13 | 148 | 150 | 34 | 152 | 154 | 37 |
| 14 | 148 | 150 | 54 | 152 | 154 | 57 |
| 15 | 148 | 150 | 74 | 152 | 154 | 77 |

In embodiments of the invention set forth in Table H, preferably the consensus sequence as set forth as SEQ ID NO:148 is a sequence as set forth as SEQ ID NO:149. In embodiments of the invention set forth in Table H, preferably the consensus sequence as set forth as SEQ ID NO:150 is a sequence as set forth as SEQ ID NO:151. In embodiments of the invention set forth in Table H, preferably the consensus sequence as set forth as SEQ ID NO:152 is a sequence as set forth as SEQ ID NO:153. In embodiments of the invention set forth in Table H, preferably the consensus sequence as set forth as SEQ ID NO:154 is a sequence as set forth as SEQ ID NO:155. In some embodiments, sequences substantially homologous to the specific sequences recited in Table H may be employed instead of the specific sequences themselves.

In some embodiments, the antibody of the invention comprises at least one heavy chain variable region that comprises three CDRs and at least one light chain variable region that comprises three CDRs, wherein said heavy chain variable region comprises a VH CDR1 that has the amino acid sequence of SEQ ID NO:148 or preferably SEQ ID NO:149 and a VH CDR2 that has the amino acid sequence of SEQ ID NO:150 or preferably SEQ ID NO:151 and/or (preferably “and”) wherein said light chain variable region comprises a VL CDR1 that has the amino acid sequence of SEQ ID NO:152 or preferably SEQ ID NO:153 and VL CDR2 that has the amino acid sequence of SEQ ID NO:154 or preferably SEQ ID NO:155.

In some such embodiments, preferably the light chain variable region comprises a VH CDR3 that has the amino acid sequence of SEQ ID NO:34 and a VL CDR3 that has the amino acid sequence of SEQ ID NO:37, or the light chain variable region comprises a VH CDR3 that has the amino acid sequence of SEQ ID NO:54 and a VL CDR3 that has the amino acid sequence of SEQ ID NO:57, or the light chain variable region comprises a VH CDR3 that has the amino acid sequence of SEQ ID NO:74 and a VL CDR3 that has the amino acid sequence of SEQ ID NO:77. In some embodiments, sequences substantially homologous to the specific sequences recited in this paragraph may be employed instead of the specific sequences themselves.

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:32 or SEQ ID NO: 52, or a sequence substantially homologous thereto,

(b) a VH CDR2 that has the amino acid sequence of SEQ ID NO: 33 or SEQ ID NO:53, or a sequence substantially homologous thereto, and

(c) a VH CDR3 that has the amino acid sequence of SEQ ID NO:34, or a sequence substantially homologous thereto; and/or (preferably “and”) wherein said light chain variable region comprises:

(d) a VL CDR1 that has the amino acid sequence of SEQ ID NO:35 or SEQ ID NO:55, or a sequence substantially homologous thereto,

(e) a VL CDR2 that has the amino acid sequence of SEQ ID NO:36 or SEQ ID NO:56, or a sequence substantially homologous thereto, and

(f) a VL CDR3 that has the amino acid sequence of SEQ ID NO:37 or SEQ ID NO: 57, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

Certain preferred combinations of VH CDR sequences and VL CDR sequences are set forth in each of the rows numbered 1-2 in Table I below:

TABLE I

| | VH | VH | VH | VL | VL | VL |
|---|---|---|---|---|---|---|
| | CDR1 | CDR2 | CDR3 | CDR1 | CDR2 | CDR3 |
| | (SEQ | (SEQ | (SEQ | (SEQ | (SEQ | (SEQ |
| | ID NO:) | ID NO:) | ID NO:) | ID NO:) | ID NO:) | ID NO:) |
| 1 | 32 | 33 | 34 | 35 | 36 | 37 |
| 2 | 52 | 53 | 34 | 55 | 56 | 57 |

In some embodiments, sequences substantially homologous to the specific sequences recited in Table I may be employed instead of the specific sequences themselves.

In some embodiments, the antibody of the invention comprises at least one heavy chain variable region that comprises three CDRs and at least one light chain variable region that comprises three CDRs, wherein said light chain variable region comprises a VH CDR3 that has the amino acid sequence of SEQ ID NO:34 (or a sequence substantially homologous thereto).

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:32 or SEQ ID NO: 72, or a sequence substantially homologous thereto,

(b) a VH CDR2 that has the amino acid sequence of SEQ ID NO: 33 or SEQ ID NO:73, or a sequence substantially homologous thereto, and

(c) a VH CDR3 that has the amino acid sequence of SEQ ID NO:34 or SEQ ID NO:74, or a sequence substantially homologous thereto; and/or (preferably “and”)

wherein said light chain variable region comprises:

(d) a VL CDR1 that has the amino acid sequence of SEQ ID NO:35 or SEQ ID NO:75, or a sequence substantially homologous thereto,

(e) a VL CDR2 that has the amino acid sequence of SEQ ID NO:36, or a sequence substantially homologous thereto, and

(f) a VL CDR3 that has the amino acid sequence of SEQ ID NO:37 or SEQ ID NO: 77, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

Certain preferred combinations of VH CDR sequences and VL CDR sequences are set forth in each of the rows numbered 1-2 in Table J below:

TABLE J

| | VH | VH | VH | VL | VL | VL |
|---|---|---|---|---|---|---|
| | CDR1 | CDR2 | CDR3 | CDR1 | CDR2 | CDR3 |
| | (SEQ | (SEQ | (SEQ | (SEQ | (SEQ | (SEQ |
| | ID NO:) | ID NO:) | ID NO:) | ID NO:) | ID NO:) | ID NO:) |
| 1 | 32 | 33 | 34 | 35 | 36 | 37 |
| 2 | 72 | 73 | 74 | 75 | 36 | 77 |

In some embodiments, sequences substantially homologous to the specific sequences recited in Table J may be employed instead of the specific sequences themselves.

In some embodiments, the antibody of the invention comprises at least one heavy chain variable region that comprises three CDRs and at least one light chain variable region that comprises three CDRs, wherein said light chain variable region comprises a VL CDR2 that has the amino acid sequence of SEQ ID NO:36 (or a sequence substantially homologous thereto).

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:32, SEQ ID NO:52 or SEQ ID NO:72, or a sequence substantially homologous thereto,

(b) a VH CDR2 that has the amino acid sequence of SEQ ID NO: 33, SEQ ID NO:53, or SEQ ID NO:73, or a sequence substantially homologous thereto, and

(c) a VH CDR3 that has the amino acid sequence of SEQ ID NO:34, SEQ ID NO:54 or SEQ ID NO:74, or a sequence substantially homologous thereto; and/or (preferably “and”) wherein said light chain variable region comprises:

(d) a VL CDR1 that has the amino acid sequence of SEQ ID NO:35, SEQ ID NO:55 or SEQ ID NO:75, or a sequence substantially homologous thereto,

(e) a VL CDR2 that has the amino acid sequence of SEQ ID NO:36, SEQ ID NO:56 or SEQ ID NO:76, or a sequence substantially homologous thereto, and

(f) a VL CDR3 that has the amino acid sequence of SEQ ID NO:37, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

Certain preferred combinations of VH CDR sequences and VL CDR sequences are set forth in each of the rows numbered 1-3 in Table K below:

TABLE K

| | VH | VH | VH | VL | VL | VL |
|---|---|---|---|---|---|---|
| | CDR1 | CDR2 | CDR3 | CDR1 | CDR2 | CDR3 |
| | (SEQ | (SEQ | (SEQ | (SEQ | (SEQ | (SEQ |
| | ID NO:) | ID NO:) | ID NO:) | ID NO:) | ID NO:) | ID NO:) |
| 1 | 32 | 33 | 34 | 35 | 36 | 37 |
| 2 | 52 | 53 | 54 | 55 | 56 | 37 |
| 3 | 72 | 73 | 74 | 75 | 76 | 37 |

In some embodiments, sequences substantially homologous to the specific sequences recited in Table K may be employed instead of the specific sequences themselves.

In some embodiments, the antibody of the invention comprises at least one heavy chain variable region that comprises three CDRs and at least one light chain variable region that comprises three CDRs, wherein said light chain variable region comprises a VL CDR3 that has the amino acid sequence of SEQ ID NO:37 (or a sequence substantially homologous thereto).

In some embodiments, the antibody of the invention comprises at least one heavy chain variable region that comprises three CDRs and at least one light chain variable region that comprises three CDRs, wherein said heavy chain variable region comprises a VH CDR1 that has the amino acid sequence of SEQ ID NO:148 (or preferably SEQ ID NO:149). In some such embodiments, preferably the VL CDR1, VL CDR2, VL CDR3, VH CDR2 and VH CDR3 (e.g. combinations thereof) have amino acid sequences as defined elsewhere herein.

In some embodiments, the antibody of the invention comprises at least one heavy chain variable region that comprises three CDRs and at least one light chain variable region that comprises three CDRs, wherein said heavy chain variable region comprises a VH CDR2 that has the amino acid sequence of SEQ ID NO:150 (or preferably SEQ ID NO:151). In some such embodiments, preferably the VL CDR1, VL CDR2, VL CDR3, VH CDR1 and VH CDR3 (e.g. combinations thereof) have amino acid sequences as defined elsewhere herein.

In some embodiments, the antibody of the invention comprises at least one heavy chain variable region that comprises three CDRs and at least one light chain variable region that comprises three CDRs, wherein said light chain variable region comprises a VL CDR1 that has the amino acid sequence of SEQ ID NO:152 (or preferably SEQ ID NO:153). In some such embodiments, preferably the VL CDR2, VL CDR3, VH CDR1, VH CDR2 and VH CDR3 (e.g. combinations thereof) have amino acid sequences as defined elsewhere herein.

In some embodiments, the antibody of the invention comprises at least one heavy chain variable region that comprises three CDRs and at least one light chain variable region that comprises three CDRs, wherein said light chain variable region comprises a VL CDR2 that has the amino acid sequence of SEQ ID NO:154 (or preferably SEQ ID NO:155). In some such embodiments, preferably the VL CDR1, VL CDR3, VH CDR1, VH CDR2 and VH CDR3 (e.g. combinations thereof) have amino acid sequences as defined elsewhere herein.

In some embodiments, the antibody of the invention comprises at least one heavy chain variable region that comprises three CDRs and at least one light chain variable region that comprises three CDRs, wherein said light chain variable region comprises a VL CDR3 that has the amino acid sequence of SEQ ID NO:37 (or SEQ ID NO:57 or SEQ ID NO: 77) (or a sequence substantially homologous thereto). In some such embodiments, preferably the VL CDR1, VL CDR2, VH CDR1, VH CDR2 and VH CDR3 (e.g. combinations thereof) have amino acid sequences as defined elsewhere herein.

In some embodiments, the antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:148 preferably SEQ ID NO: 149, and/or a VH CDR2 that has the amino acid sequence of SEQ ID NO:150 preferably SEQ ID NO: 151, and/or said light chain variable region comprises a VL CDR1 that has the amino acid sequence of SEQ ID NO:152 preferably SEQ ID NO: 153, and/or a VL CDR2 that has the amino acid sequence of SEQ ID NO:154 preferably SEQ ID NO: 155. In some such embodiments, the antibody further comprises a VL CDR3 that has the amino acid sequence of SEQ ID NO:37 (or a sequence substantially homologous thereto) and/or further comprises a VH CDR3 that has the amino acid sequence of SEQ ID NO:34 or 74 (or a sequence substantially homologous thereto).

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:156 or preferably SEQ ID NO:157, or a sequence substantially homologous thereto,

(b) a VH CDR2 that has the amino acid sequence of SEQ ID NO:158 or preferably SEQ ID NO:159, or a sequence substantially homologous thereto, and

(c) a VH CDR3 that has the amino acid sequence of SEQ ID NO:94 or SEQ ID NO:114 or SEQ ID NO:134, or a sequence substantially homologous thereto; and/or (preferably “and”) wherein said light chain variable region comprises:

(d) a variable light (VL) CDR1 that has the amino acid sequence of SEQ ID NO:95 or SEQ ID NO:115 or SEQ ID NO:135, or a sequence substantially homologous thereto,

(e) a VL CDR2 that has the amino acid sequence of SEQ ID NO:96 or SEQ ID NO:116 or SEQ ID NO:136, or a sequence substantially homologous thereto, and

(f) a VL CDR3 that has the amino acid sequence of SEQ ID NO:97, SEQ ID NO:117 or SEQ ID NO:137, or a sequence substantially homologous thereto. Substantially homologous sequences are described elsewhere herein.

Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments of the present invention, the VH CDR1 has or comprises an amino acid sequence of SEQ ID NO:156 (T F G X₄ G V X₇). In these embodiments X₄ or X₇ can be any amino acid. Preferably the X₄ residue is V or M. Preferably the X₇ residue is A or G. Thus, a preferred VH CDR1 has or comprises the amino acid sequence of SEQ ID NO:157. For example, preferred VH CDR1 sequences of this embodiment have or comprise SEQ ID NOs: 92, 112 or 132.

In some embodiments of the present invention, the VH CDR2 has or comprises an amino acid sequence of SEQ ID NO: 158 (H I W W D D D K N Y X₁₁ P A L K S). In these embodiments, X₁₁ can be any amino acid. Preferably X₁₁ is D or F. Thus, a preferred VH CDR2 has or comprises the amino acid sequence of SEQ ID NO: 159. For example, preferred VH CDR2 sequences of this embodiment have or comprise SEQ ID NOs: 93, 113 or 133.

Certain preferred combinations of VH CDR sequences and VL CDR sequences are set forth in each of the rows numbered 1-9 in Table M below:

TABLE M

| | VH | VH | VH | VL | VL | VL |
|---|---|---|---|---|---|---|
| | CDR1 | CDR2 | CDR3 | CDR1 | CDR2 | CDR3 |
| | (SEQ | (SEQ | (SEQ | (SEQ | (SEQ | (SEQ |
| | ID NO:) | ID NO:) | ID NO:) | ID NO:) | ID NO:) | ID NO:) |
| 1 | 156 | 93 | 94 | 95 | 96 | 97 |
| 2 | 156 | 113 | 114 | 115 | 116 | 117 |
| 3 | 156 | 133 | 134 | 135 | 136 | 137 |
| 4 | 92 | 158 | 94 | 95 | 96 | 97 |
| 5 | 112 | 158 | 114 | 115 | 116 | 117 |
| 6 | 132 | 158 | 134 | 135 | 136 | 137 |
| 7 | 156 | 158 | 94 | 95 | 96 | 97 |
| 8 | 156 | 158 | 114 | 115 | 116 | 117 |
| 9 | 156 | 158 | 134 | 135 | 136 | 137 |

In embodiments of the invention set forth in Table M, preferably the consensus sequence as set forth as SEQ ID NO:156 is a sequence as set forth as SEQ ID NO:157. In embodiments of the invention set forth in Table M, preferably the consensus sequence as set forth as SEQ ID NO:158 is a sequence as set forth as SEQ ID NO:159. In some embodiments, sequences substantially homologous to the specific sequences recited in Table M may be employed instead of the specific sequences themselves.

In some embodiments, the antibody of the invention comprises at least one heavy chain variable region that comprises three CDRs and at least one light chain variable region that comprises three CDRs, wherein said heavy chain variable region comprises a VH CDR1 that has the amino acid sequence of SEQ ID NO:156 or preferably SEQ ID NO:157 and a VH CDR2 that has the amino acid sequence of SEQ ID NO:158 or preferably SEQ ID NO:159. In some such embodiments, preferably the heavy chain variable region comprises a VH CDR3 that has the amino acid sequence of SEQ ID NO:94, and/or (preferably “and”) the light chain variable region comprises a VL CDR1 that has the amino acid sequence of SEQ ID NO:95, a VL CDR2 that has the amino acid sequence of SEQ ID NO:96 and a VL CDR3 that has the amino acid sequence of SEQ ID NO:96; or the heavy chain variable region comprises a VH CDR3 that has the amino acid sequence of SEQ ID NO:114, and/or (preferably “and”) the light chain variable region comprises a VL CDR1 that has the amino acid sequence of SEQ ID NO: 115, a VL CDR2 that has the amino acid sequence of SEQ ID NO: 116 and a VL CDR3 that has the amino acid sequence of SEQ ID NO: 117; or the heavy chain variable region comprises a VH CDR3 that has the amino acid sequence of SEQ ID NO:134, and/or (preferably “and”) the light chain variable region comprises a VL CDR1 that has the amino acid sequence of SEQ ID NO:135, a VL CDR2 that has the amino acid sequence of SEQ ID NO:136 and a VL CDR3 that has the amino acid sequence of SEQ ID NO:137.

In some embodiments, sequences substantially homologous to the specific sequences recited in this paragraph may be employed instead of the specific sequences themselves.

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO: 112, or a sequence substantially homologous thereto,

(b) a VH CDR2 that has the amino acid sequence of SEQ ID NO:113, or a sequence substantially homologous thereto, and

(c) a VH CDR3 that has the amino acid sequence of SEQ ID NO:114, or a sequence substantially homologous thereto; and/or (preferably “and”) wherein said light chain variable region comprises:

(d) a VL CDR1 that has the amino acid sequence of SEQ ID NO:115 or SEQ ID NO:135, or a sequence substantially homologous thereto,

(e) a VL CDR2 that has the amino acid sequence of SEQ ID NO:116 or SEQ ID NO:136, or a sequence substantially homologous thereto, and

(f) a VL CDR3 that has the amino acid sequence of SEQ ID NO:117 or SEQ ID NO: 137, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

Certain preferred combinations of VH CDR sequences and VL CDR sequences are set forth in each of the rows numbered 1-2 in Table N below:

TABLE N

| | VH | VH | VH | VL | VL | VL |
|---|---|---|---|---|---|---|
| | CDR1 | CDR2 | CDR3 | CDR1 | CDR2 | CDR3 |
| | (SEQ | (SEQ | (SEQ | (SEQ | (SEQ | (SEQ |
| | ID NO:) | ID NO:) | ID NO:) | ID NO:) | ID NO:) | ID NO:) |
| 1 | 112 | 113 | 114 | 115 | 116 | 117 |
| 2 | 112 | 113 | 114 | 135 | 136 | 137 |

In some embodiments, sequences substantially homologous to the specific sequences recited in Table N may be employed instead of the specific sequences themselves.

In some embodiments, the antibody of the invention comprises at least one heavy chain variable region that comprises three CDRs and at least one light chain variable region that comprises three CDRs, wherein said heavy chain variable region comprises a VH CDR1 that has the amino acid sequence of SEQ ID NO:112 (or a sequence substantially homologous thereto), a VH CDR2 that has the amino acid sequence of SEQ ID NO:113 (or a sequence substantially homologous thereto) and a VH CDR3 that has the amino acid sequence of SEQ ID NO:114 (or a sequence substantially homologous thereto).

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:92 or SEQ ID NO: 112, or a sequence substantially homologous thereto,

(b) a VH CDR2 that has the amino acid sequence of SEQ ID NO: 93 or SEQ ID NO:113, or a sequence substantially homologous thereto, and

(c) a VH CDR3 that has the amino acid sequence of SEQ ID NO:94 or SEQ ID NO: 114, or a sequence substantially homologous thereto; and/or

(preferably “and”) wherein said light chain variable region comprises:

(d) a VL CDR1 that has the amino acid sequence of SEQ ID NO:95 or SEQ ID NO:115, or a sequence substantially homologous thereto,

(e) a VL CDR2 that has the amino acid sequence of SEQ ID NO:96, or a sequence substantially homologous thereto, and

(f) a VL CDR3 that has the amino acid sequence of SEQ ID NO:97 or SEQ ID NO: 117, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

Certain preferred combinations of VH CDR sequences and VL CDR sequences are set forth in each of the rows numbered 1-2 in Table O below:

TABLE O

| | VH | VH | VH | VL | VL | VL |
|---|---|---|---|---|---|---|
| | CDR1 | CDR2 | CDR3 | CDR1 | CDR2 | CDR3 |
| | (SEQ | (SEQ | (SEQ | (SEQ | (SEQ | (SEQ |
| | ID NO:) | ID NO:) | ID NO:) | ID NO:) | ID NO:) | ID NO:) |
| 1 | 92 | 93 | 94 | 95 | 96 | 97 |
| 2 | 112 | 113 | 114 | 115 | 96 | 117 |

In some embodiments, sequences substantially homologous to the specific sequences recited in Table O may be employed instead of the specific sequences themselves.

In some embodiments, the antibody of the invention comprises at least one heavy chain variable region that comprises three CDRs and at least one light chain variable region that comprises three CDRs, wherein said light chain variable region comprises a VL CDR2 that has the amino acid sequence of SEQ ID NO:96 (or a sequence substantially homologous thereto).

In some embodiments, the antibody of the invention comprises at least one heavy chain variable region that comprises three CDRs and at least one light chain variable region that comprises three CDRs, wherein said heavy chain variable region comprises a VH CDR1 that has the amino acid sequence of SEQ ID NO:156 (or preferably SEQ ID NO:157). In some such embodiments, preferably the VL CDR1, VL CDR2, VL CDR3, VH CDR2 and VH CDR3 (e.g. combinations thereof) have amino acid sequences as defined elsewhere herein.

In some embodiments, the antibody of the invention comprises at least one heavy chain variable region that comprises three CDRs and at least one light chain variable region that comprises three CDRs, wherein said heavy chain variable region comprises a VH CDR2 that has the amino acid sequence of SEQ ID NO:158 (or preferably SEQ ID NO:159). In some such embodiments, preferably the VL CDR1, VL CDR2, VL CDR3, VH CDR1 and VH CDR3 (e.g. combinations thereof) have amino acid sequences as defined elsewhere herein.

In some embodiments, the antibody of the invention comprises at least one heavy chain variable region that comprises three CDRs and at least one light chain variable region that comprises three CDRs, wherein heavy chain variable region comprises a variable heavy (VH) CDR1 that has the amino acid sequence of SEQ ID NO:156 (preferably SEQ ID NO: 157), and/or a VH CDR2 that has the amino acid sequence of SEQ ID NO:158 (preferably SEQ ID NO: 159). In some such embodiments, preferably the VL CDR1, VL CDR2, VL CDR3 and VH CDR3 (e.g. combinations thereof) have amino acid sequences as defined elsewhere herein. For example, in some such embodiments, the VL CDR2 may have the amino acid sequence of SEQ ID NO:96 (or a sequence substantially homologous thereto).

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:32, or a sequence substantially homologous thereto,

(b) a VH CDR2 that has the amino acid sequence of SEQ ID NO:33, or a sequence substantially homologous thereto, and

(c) a VH CDR3 that has the amino acid sequence of SEQ ID NO:34, or a sequence substantially homologous thereto; and/or (preferably “and”) wherein said light chain variable region comprises:

(d) a variable light (VL) CDR1 that has the amino acid sequence of SEQ ID NO:35, or a sequence substantially homologous thereto,

(e) a VL CDR2 that has the amino acid sequence of SEQ ID NO:36, or a sequence substantially homologous thereto, and

(f) a VL CDR3 that has the amino acid sequence of SEQ ID NO:37, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:32, a VH CDR2 of SEQ ID NO:33, and a VH CDR3 of SEQ ID NO:34, and/or (preferably “and”) at least one light chain variable region that comprises a VL CDR1 of SEQ ID NO:35, a VL CDR2 of SEQ ID NO:36, and a VL CDR3 of SEQ ID NO:37.

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:52, or a sequence substantially homologous thereto,

(b) a VH CDR2 that has the amino acid sequence of SEQ ID NO:53, or a sequence substantially homologous thereto, and

(c) a VH CDR3 that has the amino acid sequence of SEQ ID NO:54, or a sequence substantially homologous thereto; and/or (preferably “and”) wherein said light chain variable region comprises:

(d) a variable light (VL) CDR1 that has the amino acid sequence of SEQ ID NO:55, or a sequence substantially homologous thereto,

(e) a VL CDR2 that has the amino acid sequence of SEQ ID NO:56, or a sequence substantially homologous thereto, and

(f) a VL CDR3 that has the amino acid sequence of SEQ ID NO:57, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:52, a VH CDR2 of SEQ ID NO:53, and a VH CDR3 of SEQ ID NO:54, and/or (preferably “and”) at least one light chain variable region that comprises a VL CDR1 of SEQ ID NO:55, a VL CDR2 of SEQ ID NO:56, and a VL CDR3 of SEQ ID NO:57.

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:72, or a sequence substantially homologous thereto,

(b) a VH CDR2 that has the amino acid sequence of SEQ ID NO:73, or a sequence substantially homologous thereto, and

(c) a VH CDR3 that has the amino acid sequence of SEQ ID NO:74, or a sequence substantially homologous thereto; and/or (preferably “and”) wherein said light chain variable region comprises:

(d) a variable light (VL) CDR1 that has the amino acid sequence of SEQ ID NO:75, or a sequence substantially homologous thereto,

(e) a VL CDR2 that has the amino acid sequence of SEQ ID NO:76, or a sequence substantially homologous thereto, and

(f) a VL CDR3 that has the amino acid sequence of SEQ ID NO:77, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:72, a VH CDR2 of SEQ ID NO:73, and a VH CDR3 of SEQ ID NO:74, and/or (preferably “and”) at least one light chain variable region that comprises a VL CDR1 of SEQ ID NO:75, a VL CDR2 of SEQ ID NO:76, and a VL CDR3 of SEQ ID NO:77.

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:92, or a sequence substantially homologous thereto,

(b) a VH CDR2 that has the amino acid sequence of SEQ ID NO:93, or a sequence substantially homologous thereto, and

(c) a VH CDR3 that has the amino acid sequence of SEQ ID NO:94, or a sequence substantially homologous thereto; and/or (preferably “and”) wherein said light chain variable region comprises:

(d) a variable light (VL) CDR1 that has the amino acid sequence of SEQ ID NO:95, or a sequence substantially homologous thereto,

(e) a VL CDR2 that has the amino acid sequence of SEQ ID NO:96, or a sequence substantially homologous thereto, and

(f) a VL CDR3 that has the amino acid sequence of SEQ ID NO:97, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:92, a VH CDR2 of SEQ ID NO:93, and a VH CDR3 of SEQ ID NO:94, and/or (preferably “and”) at least one light chain variable region that comprises a VL CDR1 of SEQ ID NO:95, a VL CDR2 of SEQ ID NO:96, and a VL CDR3 of SEQ ID NO:97.

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO: 112, or a sequence substantially homologous thereto,

(b) a VH CDR2 that has the amino acid sequence of SEQ ID NO:113, or a sequence substantially homologous thereto, and

(c) a VH CDR3 that has the amino acid sequence of SEQ ID NO:114, or a sequence substantially homologous thereto; and/or (preferably “and”) wherein said light chain variable region comprises:

(d) a variable light (VL) CDR1 that has the amino acid sequence of SEQ ID NO:115 or a sequence substantially homologous thereto,

(e) a VL CDR2 that has the amino acid sequence of SEQ ID NO:116, or a sequence substantially homologous thereto, and

(f) a VL CDR3 that has the amino acid sequence of SEQ ID NO:117, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:112, a VH CDR2 of SEQ ID NO:113, and a VH CDR3 of SEQ ID NO:114, and/or (preferably “and”) at least one light chain variable region that comprises a VL CDR1 of SEQ ID NO:115, a VL CDR2 of SEQ ID NO:116, and a VL CDR3 of SEQ ID NO:117.

In some embodiments, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:132, or a sequence substantially homologous thereto,

(b) a VH CDR2 that has the amino acid sequence of SEQ ID NO:133, or a sequence substantially homologous thereto, and

(c) a VH CDR3 that has the amino acid sequence of SEQ ID NO:134, or a sequence substantially homologous thereto; and/or (preferably “and”) wherein said light chain variable region comprises:

(d) a variable light (VL) CDR1 that has the amino acid sequence of SEQ ID NO:135, or a sequence substantially homologous thereto,

(e) a VL CDR2 that has the amino acid sequence of SEQ ID NO:136, or a sequence substantially homologous thereto, and

(f) a VL CDR3 that has the amino acid sequence of SEQ ID NO:137, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:132, a VH CDR2 of SEQ ID NO:133, and a VH CDR3 of SEQ ID NO:134, and/or (preferably “and”) at least one light chain variable region that comprises a VL CDR1 of SEQ ID NO:135, a VL CDR2 of SEQ ID NO:136, and a VL CDR3 of SEQ ID NO:137.

In one embodiment the invention provides an antibody comprising a VH domain that has the amino acid sequence of SEQ ID NO: 30 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 31 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:31 and/or (preferably “and”) wherein the heavy chain variable region has the amino acid sequence of SEQ ID NO:30.

In one embodiment the invention provides an antibody comprising a VH domain that has the amino acid sequence of SEQ ID NO: 50 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 51 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:51 and/or (preferably “and”) wherein the heavy chain variable region has the amino acid sequence of SEQ ID NO:50.

In one embodiment the invention provides an antibody comprising a VH domain that has the amino acid sequence of SEQ ID NO: 70 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 71 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:71 and/or (preferably “and”) wherein the heavy chain variable region has the amino acid sequence of SEQ ID NO:70.

In one embodiment the invention provides an antibody comprising a VH domain that has the amino acid sequence of SEQ ID NO: 90 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 91 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:91 and/or (preferably “and”) wherein the heavy chain variable region has the amino acid sequence of SEQ ID NO:90.

In one embodiment the invention provides an antibody comprising a VH domain that has the amino acid sequence of SEQ ID NO: 110 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 111 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO: 111 and/or (preferably “and”) wherein the heavy chain variable region has the amino acid sequence of SEQ ID NO:110.

In one embodiment the invention provides an antibody comprising a VH domain that has the amino acid sequence of SEQ ID NO: 130 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 131 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:131 and/or (preferably “and”) wherein the heavy chain variable region has the amino acid sequence of SEQ ID NO:130.

Other preferred embodiments are Ig (e.g. IgG) forms of antibodies described herein, e.g. IgG forms of the 11D6-1, 4B8-1, 7D11-1, 7G2-1, 6B2-1-1 or 6B2-1-2 antibodies (or antibodies based thereon, e.g. substantially homologous antibodies), preferably full length IgG forms. In some embodiments, the IgG is IgG₁ or IgG₂.

Thus, in some embodiments the antibody is an Ig antibody comprising CDR sequences and/or a heavy chain variable region and/or a light chain variable region as described herein. Full IgG antibodies will typically comprise two substantially identical heavy chains and two substantially identical light chains. However, as described elsewhere herein, in some cases antibodies (e.g. full IgG antibodies) may comprise two substantially identical heavy chains and two non-identical (i.e. two different light chains.

In some embodiments, antibodies based on the 11D6-1, 4B8-1, 7D11-1, 7G2-1, 6B2-1-1 or 6B2-1-2 antibody sequences set forth in Tables A, B, C, D, E and F herein are preferred.

Some examples of antibodies of the present invention are the monoclonal antibodies 11D6-1, 4B8-1, 7D11-1, 7G2-1, 6B2-1-1 or 6B2-1-2, sequences of which are shown in Tables A, B, C, D, E and F herein. The monoclonal antibodies 11D6-1, 4B8-1, 7D11-1, 7G2-1, 6B2-1-1 or 6B2-1-2 were identified using hybridoma technology, with the peptide of SEQ ID NO:5 as the immunogen peptide. The CDR domains, VH and VL domains are shown in Tables A-F herein. Antibodies comprising these CDR domains or VH and VL domains (or sequences substantially homologous thereto) are preferred aspects of the invention.

In preferred embodiments, antibodies based on the 11D6-1, 4B8-1, 7D11-1, 7G2-1, 6B2-1-1 or 6B2-1-2 antibodies (e.g. antibodies comprising CDR domains or VH and VL domains of these antibodies as set forth in Tables A-F herein, or CDR domains or VH and VL domains substantially homologous thereto) bind to (or are capable of binding to) a peptide (e.g. an isolated peptide) comprising (or consisting of) the amino acid sequence of SEQ ID NO:5 (or a conjugate comprising such a peptide).

In preferred embodiments, antibodies based on the 11D6-1, 4B8-1, 7D11-1, 7G2-1, 6B2-1-1 or 6B2-1-2 antibodies (e.g. antibodies comprising CDR domains or VH and VL domains of these antibodies as set forth in Tables A-F herein, or CDR domains or VH and VL domains substantially homologous thereto) bind to (or are capable of binding to), and preferably inhibit (or preferably are capable of inhibiting) an oncogenic mutant form of KRAS comprising a G13D mutation, for example an oncogenic mutant form of KRAS having an amino acid sequence of SEQ ID NO:9 or SEQ ID NO:12.

Certain examples of substantially homologous sequences are sequences that have at least 65% identity to the amino acid sequences disclosed. In certain embodiments, the antibodies of the invention comprise at least one light chain variable region that includes an amino acid sequence region of at least about 65%, 70% or 75%, more preferably at least about 80%, more preferably at least about 85%, more preferably at least about 90% or 95% and most preferably at least about 97%, 98% or 99% amino acid sequence identity to the amino acid sequence of SEQ ID NO:31, 51, 71, 91, 111 or 131; and/or at least one heavy chain variable region that includes an amino acid sequence region of at least about 65%, 70% or 75%, more preferably at least about 80%, more preferably at least about 85%, more preferably at least about 90% or 95% and most preferably at least about 97%, 98% or 99% amino acid sequence identity to the amino acid sequence of SEQ ID NO:30, 50, 70, 90, 110 or 130.

Other preferred examples of substantially homologous sequences are sequences containing conservative amino acid substitutions of the amino acid sequences disclosed.

Other preferred examples of substantially homologous sequences are sequences containing 1, 2 or 3, preferably 1 or 2 (more preferably 1), altered amino acids in one or more of the CDR regions disclosed. Such alterations might be conserved or non-conserved amino acid substitutions, or a mixture thereof.

In some such embodiments, preferred alterations are conservative amino acid substitutions.

In all embodiments, antibodies containing substantially homologous sequences retain the ability to bind to an oncogenic mutant form of KRAS in accordance with the invention. Preferably, antibodies containing substantially homologous sequences retain one or more (preferably all) of the properties of (e.g. described in relation to) the 11D6-1, 4B8-1, 7D11-1, 7G2-1, 6B2-1-1 or 6B2-1-2 antibodies.

Preferably, antibodies the 11D6-1, 4B8-1, 7D11-1, 7G2-1, 6B2-1-1 or 6B2-1-2, or antibodies based thereon, bind to (and preferably inhibit activity of) a G13D and/or a G12D oncogenic mutant form of KRAS (e.g. one or more of the oncogenic mutant forms of KRAS as set forth in SEQ ID NOs: 8, 9, 11 or 12). Binding and/or inhibition may be as described elsewhere herein (e.g. preferred inhibitory activities and/or preferred levels (or amounts or degrees) of inhibition, etc.).

Typically and preferably, the 11D6-1, 4B8-1, 7D11-1, 7G2-1, 6B2-1-1 or 6B2-1-2 antibodies (or antibodies based thereon) bind to an isolated peptide of SEQ ID NO:5 and/or SEQ ID NO:2. Such binding may be assessed by any suitable method (e.g. ELISA).

Further examples of substantially homologous amino acid sequences in accordance with the present invention are described elsewhere herein.

The CDRs of antibodies of the invention are preferably separated by appropriate framework regions such as those found in naturally occurring antibodies and/or effective engineered antibodies. Thus, the V_{H}, V_{L} and individual CDR sequences of the invention are preferably provided within or incorporated into an appropriate framework or scaffold to enable antigen binding. Such framework sequences or regions may correspond to naturally occurring framework regions, FR1, FR2, FR3 and/or FR4, as appropriate to form an appropriate scaffold, or may correspond to consensus framework regions, for example identified by comparing various naturally occurring framework regions. Alternatively, non-antibody scaffolds or frameworks, e.g. T cell receptor frameworks can be used.

Appropriate sequences that can be used for framework regions are well known and documented in the art and any of these may be used. Preferred sequences for framework regions are one or more of the framework regions making up the V_{H} and/or V_{L} domains of the invention, i.e. one or more of the framework regions of the 11D6-1, 4B8-1, 7D11-1, 7G2-1, 6B2-1-1 or 6B2-1-2 antibodies, as disclosed in Tables A-F, herein, or framework regions substantially homologous thereto, and in particular framework regions that allow the maintenance of antigen specificity, for example framework regions that result in substantially the same or the same 3D structure of the antibody.

In certain preferred embodiments, all four of the variable light chain (SEQ ID NOs:42, 43, 44 and 45) and/or variable heavy chain (SEQ ID NOs:38, 39, 40 and 41) framework regions (FR), as appropriate, or FR regions substantially homologous thereto, are found in the antibodies of the invention.

In other preferred embodiments, all four of the variable light chain (SEQ ID NOs:62, 63, 64 and 65) and/or variable heavy chain (SEQ ID NOs:58, 59, 60 and 61) framework regions (FR), as appropriate, or FR regions substantially homologous thereto, are found in the antibodies of the invention.

In other preferred embodiments, all four of the variable light chain (SEQ ID NOs:82, 83, 84 and 85) and/or variable heavy chain (SEQ ID NOs:78, 79, 80 and 81) framework regions (FR), as appropriate, or FR regions substantially homologous thereto, are found in the antibodies of the invention.

In other preferred embodiments, all four of the variable light chain (SEQ ID NOs:102, 103, 104 and 105) and/or variable heavy chain (SEQ ID NOs:98, 99, 100 and 101) framework regions (FR), as appropriate, or FR regions substantially homologous thereto, are found in the antibodies of the invention.

In other preferred embodiments, all four of the variable light chain (SEQ ID NOs:122, 123, 124 and 125) and/or variable heavy chain (SEQ ID NOs:118, 119, 120 and 121) framework regions (FR), as appropriate, or FR regions substantially homologous thereto, are found in the antibodies of the invention.

In other preferred embodiments, all four of the variable light chain (SEQ ID NOs:142, 143, 144 and 145) and/or variable heavy chain (SEQ ID NOs:138, 139, 140 and 141) framework regions (FR), as appropriate, or FR regions substantially homologous thereto, are found in the antibodies of the invention.

In some embodiments, VH domains and/or VL domains of the invention may additionally comprise a signal peptide at their N-terminal end (e.g. positioned immediately N-terminally with respect to the VH or VL domain). However, such signal peptides are typically absent from the antibody itself (e.g. absent from the mature antibody or isolated antibody product) as they are typically cleaved off.

In some embodiments, a VH domain comprising SEQ ID NO:30 (or sequence substantially homologous thereto) further comprises at its N-terminal end a signal peptide of SEQ ID NO:46. In some embodiments, a VL domain comprising SEQ ID NO:31 (or sequence substantially homologous thereto) further comprises at its N-terminal end a signal peptide of SEQ ID NO:47.

In some embodiments, a VH domain comprising SEQ ID NO:50 (or sequence substantially homologous thereto) further comprises at its N-terminal end a signal peptide of SEQ ID NO:66. In some embodiments, a VL domain comprising SEQ ID NO:51 (or sequence substantially homologous thereto) further comprises at its N-terminal end a signal peptide of SEQ ID NO:67.

In some embodiments, a VH domain comprising SEQ ID NO:70 (or sequence substantially homologous thereto) further comprises at its N-terminal end a signal peptide of SEQ ID NO:86. In some embodiments, a VL domain comprising SEQ ID NO:71 (or sequence substantially homologous thereto) further comprises at its N-terminal end a signal peptide of SEQ ID NO:87.

In some embodiments, a VH domain comprising SEQ ID NO:90 (or sequence substantially homologous thereto) further comprises at its N-terminal end a signal peptide of SEQ ID NO:106. In some embodiments, a VL domain comprising SEQ ID NO:91 (or sequence substantially homologous thereto) further comprises at its N-terminal end a signal peptide of SEQ ID NO:107.

In some embodiments, a VH domain comprising SEQ ID NO:110 (or sequence substantially homologous thereto) further comprises at its N-terminal end a signal peptide of SEQ ID NO:126. In some embodiments, a VL domain comprising SEQ ID NO: 111 (or sequence substantially homologous thereto) further comprises at its N-terminal end a signal peptide of SEQ ID NO:127.

In some embodiments, a VH domain comprising SEQ ID NO:130 (or sequence substantially homologous thereto) further comprises at its N-terminal end a signal peptide of SEQ ID NO:146. In some embodiments, a VL domain comprising SEQ ID NO:131 (or sequence substantially homologous thereto) further comprises at its N-terminal end a signal peptide of SEQ ID NO:147.

The binding of an antibody of the invention to an oncogenic mutant form of KRAS in accordance with the invention may be assessed by any suitable means, and the skilled person will be familiar with suitable methods (e.g. ELISA assay or using a functional assay such as a GTPase assay or a phospho-ERK inhibition assay or an apoptosis assay or a necrosis assay, e.g. as described elsewhere herein).

As indicated above, antibodies of the present invention typically inhibit activity of at least one oncogenic mutant form of KRAS in accordance with the present invention.

Preferred oncogenic mutant forms of KRAS in accordance with the present invention (and preferred groups thereof) discussed above in the context of antibody binding may also represent preferred oncogenic mutant forms of KRAS in accordance with the present invention (and preferred groups thereof) in the context of KRAS inhibition in accordance with the invention.

The inhibition of activity (inhibition of functional activity) of an oncogenic mutant form of KRAS in accordance with the present invention may be as determined by any suitable means and the skilled person is familiar with suitable assays and methods. For example, the inhibition of activity may be as determined in a GTPase assay, or a phospho-ERK inhibition assay or an apoptosis assay or a necrosis assay, for example as described elsewhere herein.

In some embodiments, inhibition of activity is any measurable or significant inhibition, more preferably a statistically significant inhibition (e.g. as compared to a control such as a control with no antibody (e.g. as compared to a “vehicle” control or as compared to a control with an antibody that does not bind to KRAS or as compared to a “no antibody” control or as compared to an isotype control).

In some embodiments, the level of inhibition (or amount of inhibition) of activity observed with (or caused by or elicited by) a control (e.g. a “vehicle” control or a control antibody that does not bind to KRAS or a “no antibody” control or an isotype control) represents (or is set as) the zero inhibition level (or zero inhibition value or 0% inhibition level or value). Thus, in some embodiments, the % inhibitions of activity discussed elsewhere herein are as compared to (or relative to) the inhibition observed with (or caused by or elicited by) a “vehicle” control (or “vehicle only” control) or with a control antibody (e.g. a control antibody that does not bind to KRAS) or with a “no antibody” control or with an isotype control.

In some embodiments, inhibition of activity is an inhibition of at least 2%, at least 5%, at least 10%, at least 15%, preferably at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100%.

In some embodiments, inhibition of activity is an inhibition of up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95% or up to 100%.

Thus, in some embodiments, inhibition of activity is an inhibition of 2%-100%, 5%-100%, 10%-100%, 15%-100%, 20%-100%, 25%-100%, 30%-100%, 35%-100%, 40%-100%, 45%-100%, 50%-100%, 55%-100%, 60%-100%, 65%-100%, 70%-100%, 75%-100%, 80%-100%, 85%-100%, 90%-100% or 95%-100%.

In some embodiments, inhibition of activity is an inhibition of 2%-75%, 5%-75%, 10%-75%, 15%-75%, 20%-75%, 25%-75%, 30%-75%, 35%-75%, 40%-75%, 45%-75%, 50%-75%, 55%-75%, 60%-75%, 65%-75% or 70%-75%.

In some embodiments, inhibition of activity is an inhibition of 2%-75%, 5%-50%, 10%-50%, 15%-50%, 20%-50%, 25%-50%, 30%-50%, 35%-50%, 40%-50% or 45%-50%.

In some embodiments, inhibition of activity is an inhibition of 2%-75%, 5%-25%, 10%-25%, 15%-25% or 20%-25%.

In some preferred embodiments, inhibition of activity is an inhibition of at least 20%, or at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100%.

As indicated above, antibodies of the invention typically inhibit activity (or are capable of inhibiting activity) of at least once oncogenic mutant form of KRAS in accordance with the invention (e.g. at least once oncogenic mutant form of KRAS having an amino acid sequence of SEQ ID NO:8, 9, 11, 12, 13, 14, 15 or 16, preferably 8, 9, 11 or 12).

In some embodiments, antibodies of the present invention inhibit activity (or are capable of inhibiting activity) of at least one oncogenic mutant form of KRAS having an amino acid sequence of SEQ ID NO:13 or 15 and at least one oncogenic mutant form of KRAS having an amino acid sequence of SEQ ID NO:14 or 16.

In some embodiments, antibodies of the present invention inhibit activity (or are capable of inhibiting activity) of at least one oncogenic mutant form of KRAS having an amino acid sequence of SEQ ID NO:8 or 11 and at least one oncogenic mutant form of KRAS having an amino acid sequence of SEQ ID NO:9 or 12.

In some embodiments, the inhibitions (e.g. % inhibitions) discussed herein are as determined when the antibody is used at a concentration in the micromolar (μM) or nanomolar (nM) range, preferably the nanomolar (nM) range. Thus, in some embodiments, the above inhibitions are as determined when the antibody is used at a concentration of ≤10 μM, ≤55 μM, ≤1 μM, ≤900 nM, ≤800 nM, ≤700 nM, ≤600 nM, ≤500 nM, ≤400 nM, ≤300 nM, ≤200 nM, ≤100 nM, ≤75 nM, ≤50 nM, ≤25 nM, ≤10 nM or ≤5 nM. Thus, in some embodiments, the inhibitions (e.g. % inhibitions) are as determined when the antibody is used at a concentration of 1 nM-≤10 μM, for example 1 nM-1 μM, 1 nM-500 nM, 1 nM to 200 nM, 1 nM-100 nM, 1 nM-50 nM. In some embodiments, the inhibitions (e.g. % inhibitions) are as determined when the antibody is used at a concentration of 20 nM≤10 μM, for example 20 nM-1 μM, 20 nM-500 nM, 20 nM to 200 nM, 20 nM-100 nM, 20 nM-≤50 nM. In some embodiments, the inhibitions (e.g. % inhibitions) are as determined when the antibody is used at a concentration of 100 nM-≤10 μM, for example 100 nM-1 μM, 100 nM-500 nM, 100 nM to 200 nM or 100 nM to 250 nM.

In some embodiments, the inhibitions (e.g. % inhibitions) discussed herein are as determined when the antibody is used at a concentration of ≤100 μg/ml, ≤50 μg/ml, ≤25 μg/ml, ≤20 μg/ml, ≤15 μg/ml, ≤10 μg/ml, ≤5 μg/ml, ≤4 μg/ml, ≤3 μg/ml, ≤2 μg/ml or ≤1 μg/ml. In some embodiments, the inhibitions (e.g. % inhibitions) are as determined when the antibody is used at a concentration of 1 μg/ml-100 μg/ml, 5 μg/ml-100 μg/ml, 10 μg/ml-100 μg/ml, 20 μg/ml-100 μg/ml 25 μg/ml-100 μg/ml, 50 μg/ml-100 μg/ml. In some embodiments, the inhibitions (e.g. % inhibitions) are as determined when the antibody is used at a concentration of 1 μg/ml-25 μg/ml, 5 μg/ml-25 μg/ml, 10 μg/ml-25 μg/ml or 15 μg/ml-25 μg/ml. In some embodiments, the inhibitions (e.g. % inhibitions) are as determined when the antibody is used at a concentration of 4 μg/ml-25 μg/ml (e.g. 4 μg/ml or 10 μg/ml or 25 μg/ml).

In some embodiments, the inhibitions (e.g. % inhibitions) and concentrations apply when the antibody is a polyclonal antibody (e.g. a rabbit polyclonal antibody). In some embodiments, the inhibitions (e.g. % inhibitions) and concentrations apply when the antibody is a monoclonal antibody.

In some embodiments, the inhibition of activity of an oncogenic mutant form of KRAS in accordance with the invention is the inhibition of GTPase activity (i.e. inhibition of the ability of an oncogenic mutant form of KRAS in accordance with the invention to convert (or hydrolyze) GTP to GDP). Thus, in some embodiments, an antibody of the invention inhibits (or is capable of inhibiting) GTPase activity of an oncogenic mutant form of KRAS in accordance with the invention. Thus, inhibition of activity by an antibody of the invention may be as determined (or as assessed) in a GTPase assay, e.g. as described elsewhere herein.

KRAS is a small GTPase. Wild-type KRAS typically acts as a molecular switch, alternating between an active (GTP-bound) form and an inactive (GDP-bound) form. Wild-type KRAS binds and hydrolyses GTP to GDP to switch between the active and inactive forms. Oncogenic mutant forms of KRAS in accordance with the present invention (G12 or G13 mutant forms of KRAS) are typically fixed in a constitutive “active” or “on” state due to an impaired ability to cycle to the inactive form. Without wishing to be bound by theory, this leads to persistent protein-protein interactions with effector proteins in the KRAS-effector signalling pathways (e.g. the MAPK pathway) that promote tumourigenesis, cancer cell survival and proliferation.

By inhibiting the GTPase activity of an oncogenic mutant form of KRAS in accordance with the present invention with an antibody of the invention, the downstream effector signalling pathways (e.g. the MAPK pathway) are typically inhibited (e.g. signalling is reduced). As will be evident from the above discussion, a reduction (or inhibition) of GTPase activity of an oncogenic mutant form of KRAS would be of benefit (e.g. therapeutic benefit) in the context of a cancer treatment.

As discussed above, antibodies of the invention may inhibit (or be capable of inhibiting) GTPase activity of an oncogenic mutant form of KRAS in accordance with the invention (e.g. as compared to a control, such as a “vehicle only” control, or such as a “no antibody” control, or such as a control antibody that does not bind to KRAS). Thus, the activity of said oncogenic mutant form of KRAS inhibited in accordance with the present invention may be GTPase activity.

In some embodiments, GTPase activity may be inhibited by at least 5%, at least 10%, at least 20%, at least 30%, at least 40%, at least 50%, at least 60%, at least 70%, at least 80%, at least 90% or at least 95%, or even 100% (e.g. as compared to a control, such as a “vehicle only” control, or such as a “no antibody” control, or such as a control antibody that does not bind to KRAS, or such as an isotype control).

GTPase activity (and inhibition of GTPase activity) may be assessed (or be as assessed) by any appropriate method or assay and the skilled person will be familiar with suitable methods and assays. GTPase assays are well-known in the art and also described herein.

For example, GTPase activity of a relevant KRAS protein (and inhibition of GTPase activity) may be assessed (or be as assessed) by an assay in which a relevant KRAS protein (e.g. an oncogenic mutant form of KRAS in accordance with the invention) is contacted with (or incubated with) a phosphate sensor (phosphate sensors are well-known in the art), GTP and an antibody under investigation, and the conversion of GTP to GDP is assessed (or monitored).

In some embodiments, in a GTPase assay the KRAS protein is a recombinant protein (e.g. a bacterially expressed, and purified, recombinant KRAS protein). Typically, the phosphate sensor is a phosphate binding molecule (e.g. a phosphate binding protein) that is modified with (or comprises or carries) a fluorophore. Typically, as a phosphate sensor binds free inorganic phosphate (e.g. as released by the conversion of GTP (guanosine triphosphate) to GDP (guanosine diphosphate) its fluorescence (or fluorescence intensity) increases. Thus, the fluorescence of the phosphate sensor can provide a report of GTPase activity (or inhibition thereof). An exemplary phosphate sensor is the phosphate sensor of Thermo Fisher Scientific, Cat No. #PV4407.

Typically, the KRAS protein, GTP, phosphate sensor and antibody under investigation (or relevant control) may be incubated together in wells of a microtitre plate (e.g. a 384 well plate) and fluorescence measured after, or over (or during), a given period of time (e.g. 4 hours) in a microtitre plate reader (e.g. excitation: 430 nm; emission: 450 nm).

In a preferred GTPase assay, an antibody under investigation is incubated with (or brought into contact with or mixed with) 1 g/l bacterially produced and purified KRAS protein, 1 μM phosphate sensor (preferably Thermo Fisher Scientific phosphate sensor, Cat No. #PV4407) and 6 μM GTP, in an appropriate buffer (e.g. a buffer having the composition 50 mM Tris, 100 mM NaCl, 10 mM MgCl₂, 1 mM EDTA, 0.01% Triton X-100 and 1 mM DTT) in wells of a microtitre plate and fluorescence (and thus KRAS activity) is measured after, or over (or during), 4 hours in a microtitre plate reader (e.g. excitation: 430 nm; emission: 450 nm).

A particularly preferred GTPase assay is described in the Example section herein.

A reduction (or inhibition or lowering) of KRAS GTPase activity by an antibody of the invention, e.g. as compared to control (such as a “vehicle only” control, or such as a “no antibody” control, or such as a control antibody that does not bind to KRAS, or such as an isotype control) is typically indicative that the antibody inhibits GTPase activity of the relevant KRAS protein.

In some embodiments, the inhibition of activity is characterised by (or results in, or leads to, or is evidenced by, or is reported by, or is as determined by) the inhibition of ERK (extracellular signal-regulated kinase) phosphorylation in a cell (or cell line) that expresses an oncogenic mutant form of KRAS in accordance with the invention. Alternatively viewed, a reduction in ERK phosphorylation in cells (or in a cell line) that express an oncogenic mutant form of KRAS in accordance with the invention caused by an antibody of the invention is typically indicative that said antibody inhibits (inhibits activity of) an oncogenic mutant form of KRAS in accordance with the invention. Thus, in some embodiments, an antibody of the invention inhibits or reduces (or is capable of inhibiting or reducing) ERK phosphorylation in cells that express an oncogenic mutant form of KRAS in accordance with the invention. Thus, inhibition of activity by an antibody of the invention may be as determined (or as assessed) in phospho-ERK assay, e.g. as described elsewhere herein.

The MAPK (MAP kinase) pathway is a major signal transduction cascade that regulates cell growth. Activity of the MAPK pathway is often upregulated in cancer. Under normal physiology, activation of the MAPK pathway occurs following the binding of growth factors to their cognate receptors, and the recruitment of a small Ras GTPase, e.g. KRAS, which then recruits the serine threonine kinase RAF. This, in turn, activates MEK, which phosphorylates ERK, the major effector kinase of the pathway. ERK then phosphorylates and activates multiple cytoplasmic substrates and transcription factors. When activated in cancer, signaling through the MAPK pathway contributes to tumour progression. For example, when activated in cancer the MAPK pathway contributes to tumour progression by the maintenance of activated cyclin D1/CDK4 complexes (which increases cell growth), the suppression of pro-apoptotic molecules such as BIM (which increases cell survival), and through direct regulation of the cytoskeleton (which contributes to invasion and metastasis).

By inhibiting the activity of an oncogenic mutant form of KRAS in accordance with the present invention with an antibody of the invention, the downstream phosphorylation of ERK is typically inhibited (i.e. levels or amounts of ERK phosphorylation, i.e. phospho-ERK, may be reduced). As will be evident from the above discussion, a reduction in ERK phosphorylation (a reduction in phosphor-ERK) would be of benefit (e.g. therapeutic benefit) in the context of a cancer treatment.

Thus, in some embodiments, antibodies of the invention may inhibit or reduce (or be capable of inhibiting or reducing) ERK phosphorylation in cancer cells or cancer cell lines that express an oncogenic mutant form of KRAS that has a G12 mutation in accordance with the invention (e.g. an oncogenic mutant form of KRAS having an amino acid sequence of SEQ ID NO:13 or SEQ ID NO:15) or may inhibit or reduce (or be capable of inhibiting or reducing) ERK phosphorylation in cancer cells or cancer cell lines that express an oncogenic mutant form of KRAS that has a G13 mutation in accordance with the invention (e.g. an oncogenic mutant form of KRAS having an amino acid sequence of SEQ ID NO:14 or SEQ ID NO:16). In some preferred embodiments, antibodies of the invention may inhibit or reduce (or be capable of inhibiting or reducing) ERK phosphorylation in cancer cells or cancer cell lines that express an oncogenic mutant form of KRAS that has a G13 mutation in accordance with the invention (e.g. an oncogenic mutant form of KRAS having an amino acid sequence of SEQ ID NO:14 or SEQ ID NO:16).

In some embodiments, antibodies of the invention may inhibit or reduce (or be capable of inhibiting or reducing) ERK phosphorylation in cancer cells or cancer cell lines that express an oncogenic mutant form of KRAS that has a G13 mutation in accordance with the invention (e.g. an oncogenic mutant form of KRAS having an amino acid sequence of SEQ ID NO:14 or SEQ ID NO:16) and in cancer cells or cancer cell lines that express an oncogenic mutant form of KRAS that has a G12 mutation in accordance with the invention (e.g. an oncogenic mutant form of KRAS having an amino acid sequence of SEQ ID NO:13 or SEQ ID NO:15).

In some embodiments, antibodies of the invention may inhibit or reduce (or be capable of inhibiting or reducing) ERK phosphorylation in cancer cells or cancer cell lines that express an oncogenic mutant form of KRAS that has a G12D mutation in accordance with the invention (e.g. an oncogenic mutant form of KRAS having an amino acid sequence of SEQ ID NO:8 or SEQ ID NO:11) or may inhibit or reduce (or be capable of inhibiting or reducing) ERK phosphorylation in cancer cells or cancer cell lines that express an oncogenic mutant form of KRAS that has a G13D mutation in accordance with the invention (e.g. an oncogenic mutant form of KRAS having an amino acid sequence of SEQ ID NO:9 or SEQ ID NO:12).

In some preferred embodiments, antibodies of the invention may inhibit or reduce (or be capable of inhibiting or reducing) ERK phosphorylation in cancer cells or cancer cell lines that express an oncogenic mutant form of KRAS that has a G13D mutation in accordance with the invention (e.g. an oncogenic mutant form of KRAS having an amino acid sequence of SEQ ID NO:9 or SEQ ID NO:12).

In some embodiments, antibodies of the invention may inhibit or reduce (or be capable of inhibiting or reducing) ERK phosphorylation in cancer cells or cancer cell lines that express an oncogenic mutant form of KRAS that has a G12D mutation in accordance with the invention (e.g. an oncogenic mutant form of KRAS having an amino acid sequence of SEQ ID NO:8 or SEQ ID NO:11) and inhibit or reduce (or be capable of inhibiting or reducing) ERK phosphorylation in cancer cells or cancer cell lines that express an oncogenic mutant form of KRAS that has a G13D mutation in accordance with the invention (e.g. an oncogenic mutant form of KRAS having an amino acid sequence of SEQ ID NO:9 or SEQ ID NO:12).

In some embodiments, the inhibition (or reduction) of ERK phosphorylation in cells (e.g. cancer cells or cancer cell lines) that express an oncogenic mutant form of KRAS in accordance with the invention may be any measurable inhibition (or reduction or decrease), preferably a significant or statistically significant inhibition (e.g. as compared to a control (e.g. as compared to the level of ERK phosphorylation caused by, or in the presence of, a negative control), such as a “vehicle only” control, or such as a “no antibody” control, or such as a control antibody or isotype control that does not bind to KRAS). In some embodiments, a preferred control is an isotype control (e.g. in an embodiment where an antibody of the invention is rabbit polyclonal antibody the isotype control can be rabbit IgG). The skilled person would be familiar with appropriate controls (e.g. isotype controls) and any appropriate control could be used.

In some embodiments, the inhibition (or reduction or decrease) in ERK phosphorylation in cells (e.g. cancer cells or cancer cell lines) that express an oncogenic mutant form of KRAS in accordance with the invention may be an inhibition (or reduction or decrease) of at least 5%, at least 10%, at least 15%, preferably at least 20%, at least 25%, at least 30%, at least 40%, at least 50%, or at least 60%, at least 70%, at least 80%, at least 90% or even 100% (e.g. as compared to a control (e.g. as compared to the level or amount of inhibition of ERK-phosphorylation caused by, or in the presence of, a control), such as a “vehicle only” control, or such as a “no antibody” control, or such as a control antibody or isotype control that does not bind to KRAS). Isotype controls are typically preferred.

In some embodiments, the % inhibition of ERK phosphorylation may be an inhibition of up to 20%, or up to 25%, or up to 30%, or up to 40%, or up to 50%, or up to 60%, or up to 70%, or up to 80%, or up to 90%, or up to 100%. In some embodiments, the % inhibition of ERK phosphorylation may be an inhibition of 5%-100%, 5%-90%, 5%-80%, 5%-70%, 5%-60%, 5%-50%, 5%-40%, 5%-30%, 5%-20%, 10%-100%, 10%-90%, 10%-80%, 10%-70%, 10%-60%, 10%-50%, 10%-40%, 10%-30%, 10%-20%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70% or 50%-60%.

In some embodiments, the above-mentioned % inhibitions of ERK phosphorylation are as determined when said antibody (e.g. a polyclonal antibody such as a rabbit polyclonal antibody) is used at a concentration of 10 nM to 500 nM, e.g. 10 nM to 200 nM or 20 nM to 175 nM, e.g. 167 nM or 67 nM or 27 nM.

In some embodiments, the above-mentioned % inhibitions of ERK phosphorylation are as compared to the inhibition of ERK phosphorylation caused by (or elicited by or observed with) an isotype control.

Thus, in some embodiments, the amount (or level) of ERK phosphorylation observed with (or measured for or determined for) a negative control (e.g. an isotype control) may represent (or be considered or be set as) the 0% inhibition of ERK phosphorylation level (or amount or value). Thus, % inhibitions of ERK phosphorylation caused by antibodies of the invention as discussed herein may be considered % inhibitions relative to such a 0% inhibition (or control level of ERK phosphorylation).

As indicated above, the inhibition of ERK phosphorylation in cancer cells or cancer cell lines that express an oncogenic mutant form of KRAS is inhibition of ERK phosphorylation in cancer cells or cancer cell lines that express an oncogenic mutant form of KRAS that has a G13 mutation (e.g. G13D mutation) in accordance with the invention and/or inhibition of ERK phosphorylation in cancer cells or cancer cell lines that express an oncogenic mutant form of KRAS that has a G12 mutation (e.g. G12D mutation) in accordance with the invention. The skilled person is familiar with such cell lines. In some embodiments, the inhibition of ERK phosphorylation is inhibition of ERK phosphorylation in the cancer cell line MDA-MB-231 (a breast cancer cell line that which expresses the G13D oncogenic mutant form of KRAS), inhibition of ERK phosphorylation in the cancer cell line SK-LU-1 (which expresses the G12D oncogenic mutant form of KRAS), inhibition of ERK phosphorylation in the cell line HCT116 (which expresses the G13D oncogenic mutant form of KRAS) and/or inhibition of ERK phosphorylation in the cancer cell line LS174T (which expresses the G12D oncogenic mutant form of KRAS). In some preferred embodiments, the inhibition of ERK phosphorylation is inhibition of ERK phosphorylation in the cancer cell line MDA-MB-231.

ERK phosphorylation, and inhibition of ERK phosphorylation, may be assessed (or be as assessed) by any appropriate method or assay and the skilled person will be familiar with suitable methods and assays. ERK phosphorylation (Phospho-ERK) assays and methods are well-known in the art and also described herein.

In some embodiments, the assay to determine (or measure) inhibition of ERK phosphorylation involves determining the phosphorylation (or phosphorylation status) at residue Thr202 and/or Tyr204 of ERK1/2.

In some embodiments, the assay to determine (or measure) ERK phosphorylation, or inhibition of ERK phosphorylation, comprises culturing cancer cells or cancer cell lines that express an oncogenic mutant form of KRAS in accordance with the invention in culture media, treating (or contacting) the cultured cells with an antibody under investigation (e.g. an antibody of the invention) or a relevant control (e.g. an isotype control), incubating the antibody (or control) treated cells in serum containing media (e.g. for 14-16 hours), replacing the media with serum-free media containing an antibody under investigation (e.g. an antibody of the invention) or a relevant control (e.g. an isotype control) and culturing the cells (e.g. for 4 hours), treating the cells (or contacting the cells) with EGF (epidermal growth factor, e.g. 10.9 ng/ml, e.g. for 10 minutes), lysing the cells (e.g. by applying lysis buffer for e.g. 10 minutes) and detecting (typically quantitatively detecting or measuring) the amount of (or level of) phosphorylated ERK (phospho-ERK1/2; phosphorylated on Thr202/Tyr204) in the cell lysate using a reagent (e.g. an antibody or antibody-based reagent) that binds to phosphorylated ERK. EGF stimulates ERK phosphorylation. Total ERK levels may also be determined in such assays. A decreased (or reduced) amount (or level) of phosphorylated ERK by (or with or after) treatment with an antibody of the invention, e.g. as compared to by (or with or after) treatment with a negative control (e.g. an isotype control), is typically indicative that the antibody of the invention inhibits ERK phosphorylation and thus is typically indicative that the antibody inhibits an oncogenic mutant form of KRAS in accordance with the invention.

In some embodiments, the assay to determine (or measure) ERK phosphorylation, or inhibition of ERK phosphorylation, comprises:

(i) plating cancer cells or cells of a cancer cell line that express an oncogenic mutant form of KRAS in accordance with the invention (e.g. MDA-MB-231 cells or cells of other cell lines described herein) at a density of 10,000 cells/well of a 384 well plate and incubating overnight at 37° C./5% CO₂;

(ii) treating (or contacting) the incubated cells of (i) with an antibody under investigation (e.g. an antibody of the invention) (e.g. 27 nM, 67 nM or 167 nM antibody) or a relevant control (e.g. an isotype control, e.g. 167 nM) and incubating the antibody (or control) treated cells in serum-containing media for 14-16 hours at 37° C./5% CO₂;

(iii) replacing the media of (ii) with serum-free media containing an antibody under investigation (e.g. an antibody of the invention) (e.g. 27 nM, 67 nM or 167 nM antibody) or a relevant control (e.g. an isotype control, e.g. 167 nM) and culturing the cells for 4 hours:

(iv) treating the cells (or contacting the cells) of (iii) with 10.9 ng/ml EGF for 10 minutes;

(v) lysing the treated cells of (iv) cells with a lysis buffer (e.g. the lysis buffer provided with the AlphaScreen® SureFire®) p-ERK1/2 (Thr202/Tyr204) Assay kit from Perkin Elmer, Cat No. TGRESB500) for 10 minutes;

(vi) quantitatively detecting (or measuring) the amount of (or level of) phosphorylated ERK (phospho-ERK1/2; phosphorylated on Thr202/Tyr204) using a reagent (e.g. an antibody or antibody-based reagent) that binds to phosphorylated ERK. Total ERK levels may also be determined in such assays (and phosphorylated ERK may be quantified as a proportion of the total ERK level). In some preferred embodiments, phosphorylated ERK is quantitatively detected using the AlphaScreen® SureFire®) p-ERK1/2 (Thr202/Tyr204) Assay kit from Perkin Elmer, Cat No. TGRESB500, according to the manufacturer's instructions. Phosphorylated ERK may quantitatively detected using a plate reader.

A decreased (or reduced) amount (or level) of phosphorylated ERK by (or with or after) treatment with an antibody of the invention as compared to by (or with or after) treatment with a negative control (e.g. an isotype control) is typically indicative that the antibody of the invention inhibits ERK phosphorylation and thus is typically indicative that the antibody inhibits an oncogenic mutant form of KRAS in accordance with the invention.

Kits and reagents for determining (or measuring) ERK phosphorylation are well-known in the art and are commercially available. As indicated above, an exemplary and preferred kit is the “AlphaScreen® SureFire®) p-ERK1/2 (Thr202/Tyr204) Assay kit” from Perkin Elmer, Cat No. TGRESB500. Preferably this kit is used according to the manufacturer's instruction.

Particularly preferred assays for determining ERK phosphorylation (and inhibition thereof) are described in the Example section herein.

In some embodiments, an assay to determine (or measure) ERK phosphorylation, or inhibition of ERK phosphorylation by an antibody of the invention, may also include a positive control (a positive control compound known to inhibit KRAS activity). In some embodiments, the positive control compound is SAH-SOS1 (stabilized alpha helices of son of sevenless 1, e.g. at 5 μM concentration). SAH-SOS1 is a polypeptide that is a known KRAS inhibitor. In some embodiments, the level (or amount) of inhibition of ERK phosphorylation by an antibody of the invention may be at least 20%, preferably at least 30%, at least 40%, at least 50%, at least 60%, at least 70%, at least 80%, at least 90%, or at least 100% or more, e.g. at least 125% or at least 150%, of the level (or amount) of inhibition of ERK phosphorylation caused by (or conferred by) SAH-SOS1.

In some embodiments, the inhibition of activity of an oncogenic mutant form of KRAS in accordance with the invention is characterised by (or results in, or leads to, or is evidenced by, or is reported by, or is as determined by) an increase in, or induction of, apoptosis in cells (or in a cell line) that express an oncogenic mutant form of KRAS in accordance with the invention. Alternatively viewed, an increase in, or induction of, apoptosis in cells (or in a cell line) that express an oncogenic mutant form of KRAS in accordance with the invention caused by an antibody of the invention is typically indicative that said antibody inhibits (inhibits activity of) an oncogenic mutant form of KRAS in accordance with the invention. Thus, in some embodiments, an antibody of the invention induces or increases (or promotes or elevates) apoptosis in cells (or in a cell line) that expresses an oncogenic mutant form of KRAS in accordance with the invention (when such cells are contacted with an antibody of the invention). Thus, inhibition of KRAS activity by an antibody of the invention may be as determined (or as assessed) in an apoptosis assay, e.g. as described elsewhere herein.

As indicated above, in cancer, activation the MAPK pathway by oncogenic mutant forms of KRAS (e.g. G12 or G13 mutant forms of KRAS in accordance with the present invention) contributes to the suppression of proapoptotic molecules such as BIM (which increases cell survival). Thus, by inhibiting an oncogenic mutant form of KRAS in accordance with the invention, MAPK pathway activation can be reduced or inhibited which can lead to the relief (or reduction) of the suppression of proapoptotic molecules, meaning that apoptosis is elevated (or increased). As will be evident from the above discussion, increasing apoptosis of cancer cells expressing an oncogenic mutant form of KRAS would be of benefit (e.g. therapeutic benefit) in the context of a cancer treatment.

In some embodiments, antibodies of the invention induce or increase (or are capable of inducing or increasing) apoptosis in cells (e.g. cancer cells or cancer cell lines) that express an oncogenic mutant form of KRAS in accordance with the invention. Typically, such an induction or increase in apoptosis would be as compared to a control, such as a “vehicle only” control, or such as a “no antibody” control, or such as a control antibody or isotype control that does not bind to KRAS.

Thus, in some embodiments, antibodies of the invention may increase or induce (or be capable of increasing or inducing) apoptosis in cancer cells or cancer cell lines that express an oncogenic mutant form of KRAS that has a G12 mutation in accordance with the invention (e.g. an oncogenic mutant form of KRAS having an amino acid sequence of SEQ ID NO:13 or SEQ ID NO:15) or may increase or induce (or be capable of increasing or inducing) apoptosis in cancer cells or cancer cell lines that express an oncogenic mutant form of KRAS that has a G13 mutation in accordance with the invention (e.g. an oncogenic mutant form of KRAS having an amino acid sequence of SEQ ID NO:14 or SEQ ID NO:16).

In some preferred embodiments, antibodies of the invention increase or induce (or are capable of increasing or inducing) apoptosis in cancer cells or cancer cell lines that express an oncogenic mutant form of KRAS that has a G12 mutation in accordance with the invention (e.g. an oncogenic mutant form of KRAS having an amino acid sequence of SEQ ID NO:13 or SEQ ID NO:15) and increase or induce (or are capable of increasing or inducing) apoptosis in cancer cells or cancer cell lines that express an oncogenic mutant form of KRAS that has a G13 mutation in accordance with the invention (e.g. an oncogenic mutant form of KRAS having an amino acid sequence of SEQ ID NO:14 or SEQ ID NO:16).

Thus, in some embodiments, antibodies of the invention may increase or induce (or be capable of increasing or inducing) apoptosis in cancer cells or cancer cell lines that express an oncogenic mutant form of KRAS that has a G12D mutation in accordance with the invention (e.g. an oncogenic mutant form of KRAS having an amino acid sequence of SEQ ID NO:8 or SEQ ID NO: 11) or may increase or induce (or be capable of increasing or inducing) apoptosis in cancer cells or cancer cell lines that express an oncogenic mutant form of KRAS that has a G13D mutation in accordance with the invention (e.g. an oncogenic mutant form of KRAS having an amino acid sequence of SEQ ID NO:9 or SEQ ID NO:12).

In some preferred embodiments, antibodies of the invention increase or induce (or are capable of increasing or inducing) apoptosis in cancer cells or cancer cell lines that express an oncogenic mutant form of KRAS that has a G12D mutation in accordance with the invention (e.g. an oncogenic mutant form of KRAS having an amino acid sequence of SEQ ID NO:8 or SEQ ID NO: 11) and increase or induce (or are capable of increasing or inducing) apoptosis in cancer cells or cancer cell lines that express an oncogenic mutant form of KRAS that has a G13D mutation in accordance with the invention (e.g. an oncogenic mutant form of KRAS having an amino acid sequence of SEQ ID NO:9 or SEQ ID NO:12).

In some embodiments, the increase in apoptosis in cells (e.g. cancer cells or cancer cell lines) that express an oncogenic mutant form of KRAS in accordance with the invention may be any measurable increase, preferably a significant or statistically significant increase (e.g. as compared to a control (e.g as compared to the level or amount of apoptosis caused by or in the presence of a control), such as a “vehicle only” control, or such as a “no antibody” control, or such as a control antibody or isotype control that does not bind to KRAS). The skilled person would be familiar with appropriate controls and any appropriate control could be used.

In some embodiments, the increase in apoptosis in cells (e.g. cancer cells or cancer cell lines) that express an oncogenic mutant form of KRAS in accordance with the invention may be an increase of at least 5%, at least 10%, at least 15%, at least, 20%, at least 25%, preferably at least 30%, at least 40%, at least 50%, at least 60%, at least 70%, at least 80%, at least 90%, at least 100%, at least 125%, at least 150%, at least 175% or at least 200% (e.g. as compared to a control (e.g. as compared to the level or amount of apoptosis caused by or in the presence of a control), such as a “vehicle only” control, or such as a “no antibody” control, or such as a control antibody or isotype control that does not bind to KRAS). In some embodiments, the % increase in apoptosis may be an increase of up to 20%, or up to 50%, or up to 100%, or up to 200% or up to 500%. In some embodiments, the % increase in apoptosis may be an increase of 5%-500%, 5%-250%, 5%-200%, 5%-100%, 5%-50%, 5%-30%, 5%-20%, 10%-500%, 10%-250%, 10%-200%, 10%-100%, 10%-50%, 10%-30%, 10%-20%, 20%-500%, 20%-250%, 20%-200%, 20%-100%, 20%-50%, 20%-30%, 30%-500%, 30%-250%, 30%-200%, 30%-100%, 30%-50%, 50%-500%, 50%-250%, 50%-200% or 50%-100%.

In some embodiments, the above-mentioned % increases in apoptosis are as determined when said antibody (e.g. a polyclonal antibody such as a rabbit polyclonal antibody) is used at a concentration of 50 nM to 500 nM, e.g. 100 nM to 300 nM, or about 200 nM (e.g. 220 nM).

In some embodiments, the above-mentioned % increases in apoptosis are as compared to the apoptosis caused by (or observed with) an isotype control. In some embodiments, the above-mentioned % increases in apoptosis are as compared to the apoptosis caused by (or observed with) a “vehicle only” control.

As indicated above, the apoptosis is apoptosis of cancer cells or cancer cell lines that express an oncogenic mutant form of KRAS that has a G12 mutation (e.g. G12D mutation) in accordance with the invention and/or apoptosis of cancer cells or cancer cell lines that express an oncogenic mutant form of KRAS that has a G13 mutation (e.g. G13D mutation) in accordance with the invention. The skilled person is familiar with such cell lines. In some embodiments, the apoptosis is apoptosis of the cancer cell line SK-LU-1 (which expresses the G12D oncogenic mutant form of KRAS), apoptosis of the cell line HCT116 (which expresses the G13D oncogenic mutant form of KRAS) and/or apoptosis of the cell line LS174T (which expresses the G12D oncogenic mutant form of KRAS). In some embodiments, the apoptosis is apoptosis of the cancer cell line SK-LU-1 or apoptosis of the cell line HCT116. In some embodiments, the apoptosis is apoptosis of the cancer cell line SK-LU-1 and apoptosis of the cell line HCT116.

Apoptosis (and increases in, or induction of, apoptosis) may be assessed (or be as assessed) by any appropriate method or assay and the skilled person will be familiar with suitable methods and assays. Apoptosis assays are well-known in the art and also described herein.

For example, in some embodiments, the apoptosis assay involves determining the exposure of phosphatidylserine (PS) to the outer leaflet of the cell membrane after the cells have been contacted with (or treated with) an antibody under investigation (e.g. an antibody of the invention) or during the cells' contact with (or treatment with) an antibody under investigation (e.g. an antibody of the invention). PS is an integral component of the plasma membrane that is actively confined to the inner membrane leaflet in healthy cells, but PS translocates to the outer leaflet of the plasma membrane during apoptosis where it can be measured.

The translocated PS may be measured by an Annexin V based molecule (e.g. a fluorescently-labelled Annexin V conjugate). Annexin V-based molecules are typically preferred probes for detecting (or determining or measuring) PS exposure to the outer leaflet because of their high, calcium-dependent affinity and selectivity for the lipid.

Thus, in some embodiments, the apoptosis caused by an antibody may be determined by (or is as determined by) a method which determines (or measures) the exposure (or translocation) of phosphatidylserine (PS) to the outer leaflet of the cell membrane, typically by detecting (or measuring) the exposed or translocated PS using an Annexin V-based reagent (e.g. a fluorescently-labelled Annexin V conjugate).

In some embodiments, the apoptosis (or level or amount of apoptosis) is the apoptosis that occurs over (or during) a set period of time (e.g. 10 hours, 24 hours, 48 hours, 52 hours, 72 hours) of contact with (or treatment with or exposure to) an antibody under investigation. In some embodiments, the apoptosis (or level or amount of apoptosis) is the apoptosis that occurs over (or during) a 52 hour period.

In some embodiments, the apoptosis (or level or amount of apoptosis) is the apoptosis that occurs over (or during) 24 hours or 48 hours of contact with (or treatment with or exposure to) an antibody under investigation.

An increase in the amount (or level) of exposed (or translocated) PS caused by an antibody of the invention (e.g. as determined or measured using an Annexin V reagent) is typically indicative that the antibody causes (or induces or increases) apoptosis and thus is typically indicative that the antibody inhibits an oncogenic mutant form of KRAS in accordance with the invention.

In some embodiments, the apoptosis assay is performed in a microtitre plate, with the apoptosis being determined (e.g. based on binding of a fluorescently labelled Annexin V conjugate to exposed PS) in a microplate reader. Such an assay may be a real-time assay.

In some other embodiments, the apoptosis assay is a flow cytometry (e.g. FACS) based assay, in which cells in medium are treated with (or contacted with or exposed to) an antibody (e.g. 3 ng/ml) under investigation (e.g. for 24 hours, 48 hours or 72 hours), washed and stained with a fluorescently labelled Annexin V molecule (e.g. Annex V Pacific Blue, ThermoFisher Cat. No. A35122), and flow cytometry (e.g. FACS) is used to quantify cell-associated fluorescence. An increase in the amount cell associated fluorescence caused by (or after treatment with) an antibody of the invention (e.g. as compared to a control (e.g as compared cell associated fluorescence caused by or in the presence of a control), such as a “vehicle only” control, or such as a “no antibody” control, or such as a control antibody or isotype control that does not bind to KRAS) is typically indicative that the antibody causes (or induces or increases) apoptosis and thus typically indicative that the antibody inhibits an oncogenic mutant form of KRAS in accordance with the invention.

In some embodiments, the apoptosis assay may be a real-time assay.

Kits and reagents for determining (or measuring) apoptosis are well-known in the art and are commercially available. An exemplary and preferred kit is the “RealTime-Glo™ Annexin V Apoptosis and Necrosis Kit” (Promega, Cat. No. JA1011). Preferably this kit is used according to the manufacturer's instruction. Particularly preferred apoptosis assays are described in the Example section herein.

In some embodiments, the inhibition of activity of an oncogenic mutant form of KRAS in accordance with the invention is characterised by (or results in, or leads to, or is evidenced by, or is reported by, or is as determined by) an increase in, or induction of, necrosis in cells (or in a cell line) that express an oncogenic mutant form of KRAS in accordance with the invention. Alternatively viewed, an increase in, or induction of, necrosis in cells (or in a cell line) that express an oncogenic mutant form of KRAS in accordance with the invention caused by an antibody of the invention is typically indicative that said antibody inhibits (inhibits activity of) an oncogenic mutant form of KRAS in accordance with the invention. Thus, in some embodiments, an antibody of the invention induces or increases (or promotes or elevates) necrosis in cells (or in a cell line) that expresses an oncogenic mutant form of KRAS in accordance with the invention (when such cells are contacted with an antibody of the invention). Thus, inhibition of KRAS activity by an antibody of the invention may be as determined (or as assessed) in a necrosis assay, e.g. as described elsewhere herein.

It is clear that increasing necrosis of cancer cells expressing an oncogenic mutant form of KRAS could be of benefit (e.g. therapeutic benefit) in the context of a cancer treatment.

In some embodiments, antibodies of the invention increase or induce (or are capable of increasing or inducing) necrosis in cells (e.g. cancer cells or cancer cell lines) that express an oncogenic mutant form of KRAS in accordance with the invention. Typically, such an increase in, or induction of, necrosis would be as compared to a control, such as a “vehicle only” control, or such as a “no antibody” control, or such as a control antibody or isotype control that does not bind to KRAS.

Thus, in some embodiments, antibodies of the invention may increase or induce (or be capable of increasing or inducing) necrosis in cancer cells or cancer cell lines that express an oncogenic mutant form of KRAS that has a G12 mutation in accordance with the invention (e.g. an oncogenic mutant form of KRAS having an amino acid sequence of SEQ ID NO:13 or SEQ ID NO:15) or may increase or induce (or be capable of increasing or inducing) necrosis in cancer cells or cancer cell lines that express an oncogenic mutant form of KRAS that has a G13 mutation in accordance with the invention (e.g. an oncogenic mutant form of KRAS having an amino acid sequence of SEQ ID NO:14 or SEQ ID NO:16).

In some preferred embodiments, antibodies of the invention increase or induce (or are capable of increasing or inducing) necrosis in cancer cells or cancer cell lines that express an oncogenic mutant form of KRAS that has a G12 mutation in accordance with the invention (e.g. an oncogenic mutant form of KRAS having an amino acid sequence of SEQ ID NO:13 or SEQ ID NO:15) and increase or induce (or are capable of increasing or inducing) necrosis in cancer cells or cancer cell lines that express an oncogenic mutant form of KRAS that has a G13 mutation in accordance with the invention (e.g. an oncogenic mutant form of KRAS having an amino acid sequence of SEQ ID NO:14 or SEQ ID NO:16).

Thus, in some embodiments, antibodies of the invention may increase or induce (or be capable of increasing or inducing) necrosis in cancer cells or cancer cell lines that express an oncogenic mutant form of KRAS that has a G12D mutation in accordance with the invention (e.g. an oncogenic mutant form of KRAS having an amino acid sequence of SEQ ID NO:8 or SEQ ID NO: 11) or may increase or induce (or be capable of increasing or inducing) necrosis in cancer cells or cancer cell lines that express an oncogenic mutant form of KRAS that has a G13D mutation in accordance with the invention (e.g. an oncogenic mutant form of KRAS having an amino acid sequence of SEQ ID NO:9 or SEQ ID NO:12).

In some preferred embodiments, antibodies of the invention increase or induce (or are capable of increasing or inducing) necrosis in cancer cells or cancer cell lines that express an oncogenic mutant form of KRAS that has a G12D mutation in accordance with the invention (e.g. an oncogenic mutant form of KRAS having an amino acid sequence of SEQ ID NO:8 or SEQ ID NO:11) and increase or induce (or are capable of increasing or inducing) necrosis in cancer cells or cancer cell lines that express an oncogenic mutant form of KRAS that has a G13D mutation in accordance with the invention (e.g. an oncogenic mutant form of KRAS having an amino acid sequence of SEQ ID NO:9 or SEQ ID NO:12).

In some embodiments, the increase in necrosis in cells (e.g. cancer cells or cancer cell lines) that express an oncogenic mutant form of KRAS in accordance with the invention may be any measurable increase, preferably a significant or statistically significant increase (e.g. as compared to a control (e.g as compared to the level or amount of necrosis caused by or in the presence of a control), such as a “vehicle only” control, or such as a “no antibody” control, or such as a control antibody or isotype control that does not bind to KRAS). The skilled person would be familiar with appropriate controls and any appropriate control could be used. In some embodiments, the increase in necrosis in cells (e.g. cancer cells or cancer cell lines) that express an oncogenic mutant form of KRAS in accordance with the invention may be an increase of at least 5%, at least 10%, at least 15%, at least, 20%, at least 25%, preferably, at least 30%, at least 40%, at least 50%, at least 60%, at least 70%, at least 80%, at least 90%, at least 100%, at least 125%, at least 150%, at least 175% or at least 200% (e.g. as compared to a control (e.g. as compared to the level or amount of necrosis caused by or in the presence of a control), such as a “vehicle only” control, or such as a “no antibody” control, or such as a control antibody or isotype control that does not bind to KRAS). In some embodiments, the % increase in necrosis may be an increase of up to 20%, or up to 50%, or up to 100%, or up to 200% or up to 500%. In some embodiments, the % increase in necrosis may be an increase of 5%-500%, 5%-250%, 5%-200%, 5%-100%, 5%-50%, 5%-30%, 5%-20%, 10%-500%, 10%-250%, 10%-200%, 10%-100%, 10%-50%, 10%-30%, 10%-20%, 20%-500%, 20%-250%, 20%-200%, 20%-100%, 20%-50%, 20%-30%, 30%-500%, 30%-250%, 30%-200%, 30%-100%, 30%-50%, 50%-500%, 50%-250%, 50%-200% or 50%-100%.

In some embodiments, the above-mentioned % increases in necrosis are as determined when said antibody (e.g. a polyclonal antibody such as a rabbit polyclonal antibody) is used at a concentration of 50 nM to 500 nM, e.g. 100 nM to 300 nM, or about 200 nM (e.g. 220 nM).

In some embodiments, the above-mentioned % increases in necrosis are as compared to the necrosis caused by (or observed with) a “vehicle only” control.

As indicated above, the necrosis is necrosis of cancer cells or cancer cell lines that express an oncogenic mutant form of KRAS that has a G12 mutation (e.g. G12D mutation) in accordance with the invention and/or necrosis of cancer cells or cancer cell lines that express an oncogenic mutant form of KRAS that has a G13 mutation (e.g. G13D mutation) in accordance with the invention. The skilled person is familiar with such cell lines. In some embodiments, the necrosis is necrosis of the cancer cell line SK-LU-1 (which expresses the G12D oncogenic mutant form of KRAS) or necrosis of the cell line HCT116 (which expresses the G13D oncogenic mutant form of KRAS). In some embodiments, the necrosis is necrosis of the cancer cell line SK-LU-1 and necrosis of the cell line HCT116.

Necrosis (and increases in necrosis) may be assessed (or be as assessed) by any appropriate method or assay and the skilled person will be familiar with suitable methods and assays. Necrosis assays are well-known in the art and also described herein.

For example, in some embodiments, the necrosis caused by an antibody may be determined by (or is as determined by) a method which determines (or measures) the ability of a cell-impermeant pro-fluorescent dye to enter cells. During necrosis the cell membrane disintegrates thus allowing such a cell-impermeant pro-fluorescent dye to enter the cell.

In some embodiments, the necrosis (or level or amount of necrosis) is the necrosis that occurs over (or during) a set period of time (e.g. 24 hours, 48 hours, 52 hours, 72 hours) of contact with (or treatment with or exposure to) an antibody under investigation. In some embodiments, the necrosis (or level or amount of apoptosis) is the necrosis that occurs over (or during) a 52 hour period.

An increase in the amount of a cell-impermeant pro-fluorescent dye that enters a cell caused by an antibody of the invention is typically indicative that the antibody causes (or induces or increases) necrosis and thus is typically indicative that the antibody inhibits an oncogenic mutant form of KRAS in accordance with the invention.

In some embodiments, the necrosis assay is performed in a microtitre plate, with the necrosis being determined (e.g. based on entry of a cell-impermeant pro-fluorescent dye into cells) in a microplate reader. Such an assay may be a real-time assay.

In some embodiments, the necrosis assay may be a real-time assay.

Kits and reagents for determining (or measuring) necrosis are well-known in the art and are commercially available. An exemplary and preferred kit is the “RealTime-Glo™ Annexin V Apoptosis and Necrosis Kit” (Promega, Cat. No. JA1011). Preferably this kit is used according to the manufacturer's instruction.

A particularly preferred necrosis assay is described in the Example section herein.

In some embodiments, antibodies of the invention may additionally bind to wild-type KRAS (e.g. KRAS having an amino acid sequence of SEQ ID NO:7 or SEQ ID NO:10). In other embodiments, antibodies of the invention do not bind additionally to wild-type KRAS (or do not bind significantly to wild-type KRAS).

In some embodiments, antibodies preferentially bind to an oncogenic mutant form of KRAS in accordance with the invention as compared to wild-type KRAS. Put another way, in some embodiments, antibodies selectively bind to an oncogenic mutant form of KRAS in accordance with the invention as compared to wild-type KRAS. For example, in some embodiments, antibodies may preferentially bind to (or selectively bind to) an oncogenic mutant form of KRAS comprising a G13D mutation (e.g. an oncogenic mutant form of KRAS having an amino acid sequence as set forth in SEQ ID NO:9 or SEQ ID NO:12) as compared to (or as opposed to) wild-type KRAS (e.g. wild-type KRAS having an amino acid sequence as set forth in SEQ ID NO:7 or SEQ ID NO:10). For example, in some embodiments, antibodies based on the 11D6-1, 4B8-1 or 7D11-1 antibodies (e.g. antibodies comprising CDR sequences, VH domain sequences or VL domain sequences of these antibodies or having substantially homologous sequences) may preferentially bind to (or selectively bind to) an oncogenic mutant form of KRAS comprising a G13D mutation (e.g. an oncogenic mutant form of KRAS having an amino acid sequence as set forth in SEQ ID NO:9 or SEQ ID NO:12) as compared to (or as opposed to) wild-type KRAS (e.g. wild-type KRAS having an amino acid sequence as set forth in SEQ ID NO:7 or SEQ ID NO:10).

In some preferred embodiments, antibodies of the present invention preferentially inhibit activity of at least one oncogenic mutant form of KRAS in accordance with the present invention as compared to wild-type KRAS. In some preferred embodiments, antibodies of the present invention selectively inhibit activity of at least one oncogenic mutant form of KRAS in accordance with the present invention as compared to wild-type KRAS. For example, in some embodiments, antibodies may preferentially inhibit (or selectively inhibit) an oncogenic mutant form of KRAS comprising a G13D mutation (e.g. an oncogenic mutant form of KRAS having an amino acid sequence as set forth in SEQ ID NO:9 or SEQ ID NO:12) as compared to (or as opposed to) wild-type KRAS (e.g. wild-type KRAS having an amino acid sequence as set forth in SEQ ID NO:7 or SEQ ID NO:10). In some embodiments, antibodies based on the 11D6-1, 4B8-1, 7D11-1, 6B2-1-1, 6B2-1-2 or 7G2-1 (preferably 11D6-1, 4B8-1 or 7D11-1) antibodies (e.g. antibodies comprising CDR sequences, VH domain sequences or VL domain sequences of these antibodies or having substantially homologous sequences) may preferentially inhibit (or selectively inhibit) an oncogenic mutant form of KRAS comprising a G13D mutation (e.g. an oncogenic mutant form of KRAS having an amino acid sequence as set forth in SEQ ID NO:9 or SEQ ID NO:12) as compared to (or as opposed to) wild-type KRAS (e.g. wild-type KRAS having an amino acid sequence as set forth in SEQ ID NO:7 or SEQ ID NO:10).

In some embodiments, antibodies of the present invention do not (or do not significantly) inhibit the activity of wild-type KRAS.

Activity may be an activity as described elsewhere herein (e.g. GTPase activity, apoptosis inducing activity, necrosis inducing activity or ERK phosphorlyation inhibitory activity). Thus, such activity may be determined (or be as determined) as described elsewhere herein (e.g. in a GTPase assay, apoptosis assay, necrosis assay and/or phospho-ERK inhibition assay).

Thus, in preferred embodiments, antibodies of the invention preferentially inhibit activity of one or more oncogenic mutant forms of KRAS in accordance with the invention as opposed to the activity of wild-type KRAS. Put another way, in some preferred embodiments, an antibody of the invention inhibits (or is capable of inhibiting) activity of one or more oncogenic mutant forms of KRAS in accordance with the invention to a greater extent than it inhibits wild-type KRAS activity. Thus, for example, in some embodiments, if a given antibody inhibits activity of one or more oncogenic mutant forms of KRAS in accordance with the invention by X %, that antibody will inhibit wild-type KRAS activity by <X %.

In some embodiments, the % inhibition of one or more oncogenic mutant forms of KRAS is at least 5% higher, but typically at least 10% higher, preferably at least 20% higher, at least 30% higher, at least 40% higher, at least 50% higher, at least 60% higher, at least 70% higher, at least 80% higher, at least 90%, at least 100%, at least, 150% or at least 200% higher than the % inhibition of wild-type KRAS.

By way of example, in some embodiments, an antibody of the invention causes (or elicits) a level (or amount) of apoptosis and/or (preferably “and”) necrosis (which as discussed elsewhere herein is indicative of KRAS inhibition) of cells (e.g. cancer cells or cancer cell lines) that express an oncogenic mutant form of KRAS that has a G12 mutation in accordance with the invention (e.g. an oncogenic mutant form of KRAS having an amino acid sequence of SEQ ID NO:13 or SEQ ID NO:15) or of cells (e.g. cancer cells or cancer cell lines) that express an oncogenic mutant form of KRAS that has a G13 mutation in accordance with the invention (e.g. an oncogenic mutant form of KRAS having an amino acid sequence of SEQ ID NO:14 or SEQ ID NO:16) that is higher (preferably significantly higher) than the level (or amount) of apoptosis and/or (preferably “and”) necrosis that said antibody causes (or elicits) in cells that express wild-type KRAS (SEQ ID NO:7 or SEQ ID NO:10).

In some embodiments, an antibody of the invention causes (or elicits) a level (or amount) of apoptosis and/or (preferably “and”) necrosis of cells (e.g. cancer cells or cancer cell lines) that express an oncogenic mutant form of KRAS that has a G12 mutation in accordance with the invention (e.g. an oncogenic mutant form of KRAS having an amino acid sequence of SEQ ID NO:13 or SEQ ID NO:15) and of cells (e.g. cancer cells or cancer cell lines) that express an oncogenic mutant form of KRAS that has a G13 mutation in accordance with the invention (e.g. an oncogenic mutant form of KRAS having an amino acid sequence of SEQ ID NO:14 or SEQ ID NO:16) that is higher (preferably significantly higher) than the level (or amount) of apoptosis and/or (preferably “and”) necrosis that said antibody causes (or elicits) in cells that express wild-type KRAS (SEQ ID NO:7 or SEQ ID NO:10).

In some embodiments, an antibody of the invention causes (or elicits) a level (or amount) of apoptosis and/or (preferably “and”) necrosis of cells (e.g. cancer cells or cancer cell lines) that express an oncogenic mutant form of KRAS that has a G12D mutation in accordance with the invention (e.g. an oncogenic mutant form of KRAS having an amino acid sequence of SEQ ID NO:8 or SEQ ID NO:11) or of cells (e.g. cancer cells or cancer cell lines) that express an oncogenic mutant form of KRAS that has a G13D mutation in accordance with the invention (e.g. an oncogenic mutant form of KRAS having an amino acid sequence of SEQ ID NO:9 or SEQ ID NO:12) that is higher (preferably significantly higher) than the level (or amount) of apoptosis and/or (preferably “and”) necrosis that said antibody causes (or elicits) in cells that express wild-type KRAS (SEQ ID NO:7 or SEQ ID NO:10).

In some embodiments, an antibody of the invention causes (or elicits) a level (or amount) of apoptosis and/or (preferably “and”) necrosis of cells (e.g. cancer cells or cancer cell lines) that express an oncogenic mutant form of KRAS that has a G13D mutation in accordance with the invention (e.g. an oncogenic mutant form of KRAS having an amino acid sequence of SEQ ID NO:9 or SEQ ID NO:12) that is higher (preferably significantly higher) than the level (or amount) of apoptosis and/or (preferably “and”) necrosis that said antibody causes (or elicits) in cells that express wild-type KRAS (SEQ ID NO:7 or SEQ ID NO:10).

In some embodiments, an antibody of the invention causes (or elicits) a level (or amount) of apoptosis and/or (preferably “and”) necrosis of cells (e.g. cancer cells or cancer cell lines) that express an oncogenic mutant form of KRAS that has a G12D mutation in accordance with the invention (e.g. an oncogenic mutant form of KRAS having an amino acid sequence of SEQ ID NO:8 or SEQ ID NO:11) and of cells (e.g. cancer cells or cancer cell lines) that express an oncogenic mutant form of KRAS that has a G13D mutation in accordance with the invention (e.g. an oncogenic mutant form of KRAS having an amino acid sequence of SEQ ID NO:9 or SEQ ID NO:12) that is higher (preferably significantly higher) than the level (or amount) of apoptosis and/or (preferably “and”) necrosis that said antibody causes (or elicits) in cells that express wild-type KRAS (SEQ ID NO:7 or SEQ ID NO:10).

In some embodiments, the higher level of apoptosis and/or necrosis caused in cells that express an oncogenic mutant form of KRAS is a level that is at least 5% higher, but typically at least 10% higher, preferably at least 20% higher, at least 30% higher, at least 40% higher, at least 50% higher, at least 60% higher, at least 70% higher, at least 80% higher, at least 90%, at least 100%, at least, 150% or at least 200% higher than the level of apoptosis and/or necrosis caused in cells that express wild-type KRAS. In some embodiments, the higher level of apoptosis and/or necrosis in cells that express an oncogenic mutant form of KRAS is a level that is up to 500% higher, up to 400% higher, up to 300% higher, up to 200% higher, up to 100% higher or up to 50% higher than the level of apoptosis and/or necrosis in cells that express wild-type KRAS. In some embodiments, the higher level of apoptosis and/or necrosis in cells that express an oncogenic mutant form of KRAS is a level that is up to 5-500% higher, 5-400% higher, 5-300% higher, 5-200% higher, 5-100% higher, 5-50% higher, 5-20% higher, 10-500% higher, 10-400% higher, 10-300% higher, 10-200% higher, 10-100% higher, 10-50% higher, 10-20% higher, 20-500% higher, 20-400% higher, 20-300% higher, 20-200% higher, 20-100% higher, 20-50% higher, 50-500% higher, 50-400% higher, 50-300% higher, 50-200% higher, or 50-100% higher than the level of apoptosis and/or necrosis in cells that express wild-type KRAS.

As discussed elsewhere herein, in some embodiments, a cancer cell line that expresses an oncogenic G12 mutant form of KRAS may be the cancer cell line SK-LU-1 (which expresses the G12D oncogenic mutant form of KRAS). In some embodiments, a cancer cell line that expresses an oncogenic G13 mutant form of KRAS may be the cancer cell line HCT116 (which expresses the G13D oncogenic mutant form of KRAS). In some embodiments, cells that express wild-type KRAS are non-malignant cells, e.g. cell line CRL-1831 (non-malignant colon epithelial cells). In some embodiments, cells that express wild-type KRAS are cancer cells (or a cancer cell line) that express wild-type KRAS, e.g. cell line NCI-H1975 (lung adenocarcinoma cells).

In some embodiments, the higher level of apoptosis and/or necrosis caused in cells (or in a cell line) that express an oncogenic mutant form of KRAS in accordance with the invention is higher than (or higher in comparison to) the level of apoptosis and/or necrosis caused in cells (or a cell line) of (or derived from) the same tissue or organ (or tissue-type or organ-type) that express wild-type KRAS.

In some embodiments, antibodies preferentially (or selectively) bind to a given oncogenic mutant form of KRAS in accordance with the invention having a given oncogenic mutant residue (e.g. a given mutant residue described elsewhere herein) at a given position in KRAS (position 12 or 13) as opposed to a different (i.e. another) oncogenic mutant form of KRAS that has a different oncogenic mutant residue (e.g. a different mutant amino acid residue as described elsewhere herein) at the corresponding position (position 12 or 13) in KRAS.

In some embodiments, antibodies of the invention may preferentially (or selectively) bind an oncogenic mutant form of KRAS having a D (aspartic acid) residue at 12 or 13 as opposed to an oncogenic mutant form of KRAS that has a different oncogenic mutant residue at the corresponding position (position 12 or 13) in KRAS. For example, in some embodiments, antibodies of the invention may preferentially bind an oncogenic mutant form of KRAS having a D (aspartic acid) residue at 12 as opposed to an oncogenic mutant form of KRAS that has a V (valine) residue or C (cysteine) residue at the corresponding position (position 12) in KRAS.

In some embodiments, an antibody of the invention may preferentially (or selectively) bind to KRAS on (or in) cells (e.g. cancer cells or cancer cell lines) that express an oncogenic mutant form of KRAS that has a given G12 mutation (mutation at position 12) in accordance with the invention (e.g. an oncogenic mutant form of KRAS having one of the potential mutant residues set forth in the amino acid sequence of SEQ ID NO:13 or SEQ ID NO:15) as opposed to cells that express an oncogenic mutant form of KRAS having a different oncogenic mutant residue at G12 (mutation at position 12) (e.g. having a different one of the potential mutant residues set forth in the amino acid sequence of SEQ ID NO:13 or SEQ ID NO:15). For example, an antibody of the invention may preferentially (or selectively) bind to KRAS on (or in) cells (e.g. cancer cells or cancer cell lines) that express an oncogenic mutant form of KRAS that has a G12D mutation (e.g. SEQ ID NO:8 or SEQ ID NO:11) as opposed to cells that express an oncogenic mutant form of KRAS having a different oncogenic mutant residue at G12 (different, i.e. non-D, mutation at position 12, (e.g. having a different, i.e. non-D, one of the potential mutant residues set forth in the amino acid sequence of SEQ ID NO:13 or SEQ ID NO:15). For example, an antibody of the invention may bind to KRAS on (or in) cells (e.g. cancer cells or cancer cell lines) that express an oncogenic mutant form of KRAS that has a G12D mutation (e.g. SEQ ID NO:8 or SEQ ID NO:11) as opposed to KRAS on (or in) cells that express an oncogenic mutant form of KRAS having a G12V or G12C mutation.

In some embodiments, an antibody of the invention may preferentially (or selectively) bind to KRAS on (or in) cells (e.g. cancer cells or cancer cell lines) that express an oncogenic mutant form of KRAS that has a given G13 mutation (mutation at position 13) in accordance with the invention (e.g. an oncogenic mutant form of KRAS having one of the potential mutant residues set forth in the amino acid sequence of SEQ ID NO:14 or SEQ ID NO:16) as opposed to cells that express an oncogenic mutant form of KRAS having a different oncogenic mutant residue at G13 (mutation at position 13). For example, an antibody of the invention may preferentially (or selectively) bind to KRAS on (or in) cells (e.g. cancer cells or cancer cell lines) that express an oncogenic mutant form of KRAS that has a G13D mutation (e.g. SEQ ID NO:9 or SEQ ID NO:12) as opposed to cells that express an oncogenic mutant form of KRAS having a different oncogenic mutant residue at G13 (different, i.e. non-D, mutation at position 13). For example, an antibody of the invention may bind to KRAS on (or in) cells (e.g. cancer cells or cancer cell lines) that express an oncogenic mutant form of KRAS that has a G13D mutation (e.g. SEQ ID NO:9 or SEQ ID NO:12) as opposed to KRAS on cells that express an oncogenic mutant form of KRAS having a G13C mutation.

In some embodiments, an antibody of the invention may preferentially (or selectively) bind to KRAS on (or in) cells (e.g. cancer cells or cancer cell lines) that express an oncogenic mutant form of KRAS that has a given G12 mutation (mutation at position 12) in accordance with the invention (e.g. an oncogenic mutant form of KRAS having one of the potential mutant residues set forth in the amino acid sequence of SEQ ID NO:13 or SEQ ID NO:15) as opposed to cells that express an oncogenic mutant form of KRAS having a different oncogenic mutant residue at G13 (mutation at position 13). For example, an antibody of the invention may preferentially (or selectively) bind to KRAS on (or in) cells (e.g. cancer cells or cancer cell lines) that express an oncogenic mutant form of KRAS that has a G12D mutation (e.g. SEQ ID NO:8 or SEQ ID NO: 11) as opposed to cells that express an oncogenic mutant form of KRAS having a different (i.e. non-D) oncogenic mutant residue at G13 (different, i.e. non-D, mutation at position 13). For example, an antibody of the invention may preferentially bind to KRAS on (or in) cells (e.g. cancer cells or cancer cell lines) that express an oncogenic mutant form of KRAS that has a G12D mutation (e.g. SEQ ID NO:8 or SEQ ID NO: 11) as opposed to KRAS on (or in) cells that express an oncogenic mutant form of KRAS having a G13X mutation (wherein X is a oncogenic mutant residue other than D, e.g. wherein X is C).

In some embodiments, an antibody of the invention may preferentially (or selectively) bind to KRAS on (or in) cells (e.g. cancer cells or cancer cell lines) that express an oncogenic mutant form of KRAS that has a given G13 mutation (mutation at position 13) in accordance with the invention (e.g. an oncogenic mutant form of KRAS having one of the potential mutant residues set forth in the amino acid sequence of SEQ ID NO:14 or SEQ ID NO:16) as opposed to cells that express an oncogenic mutant form of KRAS having a different oncogenic mutant residue at G12 (mutation at position 12). For example, an antibody of the invention may preferentially (or selectively) bind to KRAS on (or in) cells (e.g. cancer cells or cancer cell lines) that express an oncogenic mutant form of KRAS that has a G13D mutation (e.g. SEQ ID NO:9 or SEQ ID NO:12) as opposed to cells that express an oncogenic mutant form of KRAS having a different (i.e. non-D) oncogenic mutant residue at G12 (different, i.e. non-D, mutation at position 12). For example, an antibody of the invention may preferentially bind to KRAS on (or in) cells (e.g. cancer cells or cancer cell lines) that express an oncogenic mutant form of KRAS that has a G13D mutation (e.g. SEQ ID NO:9 or SEQ ID NO:12) as opposed to KRAS on (or in) cells that express an oncogenic mutant form of KRAS having a G12X mutation (wherein X is a oncogenic mutant residue other than D, e.g. wherein X is V or C or A or S or R).

Binding may be as determined by any appropriate means (e.g. as described elsewhere herein).

In some embodiments, antibodies of the invention preferentially inhibit activity of a given oncogenic mutant form of KRAS in accordance with the invention having a given oncogenic mutant residue (e.g. a mutant residue described elsewhere herein) at a given position in KRAS (position 12 or 13) as opposed to the activity of a different (i.e. another) oncogenic mutant form of KRAS that has a different oncogenic mutant residue (e.g. a different mutant amino acid residue as described elsewhere herein) at the corresponding position (position 12 or 13) in KRAS. By way of example, in some embodiments, antibodies of the invention preferentially inhibit activity of an oncogenic mutant form of KRAS having a D (aspartic acid) residue at 12 or 13 as opposed to the activity of an oncogenic mutant form of KRAS that has a different oncogenic mutant residue at the corresponding position (position 12 or 13) in KRAS. For example, in some embodiments, antibodies of the invention preferentially inhibit activity of an oncogenic mutant form of KRAS having a D (aspartic acid) residue at 12 as opposed to the activity of an oncogenic mutant form of KRAS that has a V (valine) residue or C (cysteine) residue at the corresponding position (position 12) in KRAS.

Put another way, in some preferred embodiments, an antibody of the invention inhibits (or is capable of inhibiting) activity of an oncogenic mutant form having a given oncogenic mutant residue (e.g. a mutant residue described elsewhere herein) at a given position in KRAS (position 12 or 13) to a greater extent than it inhibits a different (i.e. another) oncogenic mutant form of KRAS that has a different oncogenic mutant residue (e.g. a different mutant amino acid residue as described elsewhere herein) at the corresponding position (position 12 or 13) in KRAS. Thus, for example, in some embodiments, if a given antibody inhibits activity of an oncogenic mutant form of KRAS in accordance with the invention having a given oncogenic mutant residue (e.g. a mutant residue described elsewhere herein) at a given position in KRAS (position 12 or 13) by X %, that antibody may inhibit a different (i.e. another) oncogenic mutant form of KRAS that has a different oncogenic mutant residue (e.g. a different mutant amino acid residue as described elsewhere herein) at the corresponding position (position 12 or 13) activity by <X %.

In some embodiments, the % inhibition may be at least 5% higher, but typically at least 10% higher, preferably at least 20% higher, at least 30% higher, at least 40% higher, at least 50% higher, at least 60% higher, at least 70% higher, at least 80% higher, at least 90%, at least 100%, at least, 150% or at least 200% higher than the % inhibition of a different (i.e. another) oncogenic mutant form of KRAS that has a different oncogenic mutant residue (e.g. a different mutant amino acid residue as described elsewhere herein) at the corresponding position (position 12 or 13).

Inhibition of activity may be as described elsewhere herein (e.g. may be characterised by causing or inducing or increasing apoptosis, which may be determined as described elsewhere herein).

In some embodiments, antibodies of the invention may preferentially inhibit activity of a given oncogenic mutant form of KRAS in accordance with the invention having a given oncogenic mutant residue (e.g. a mutant residue described elsewhere herein) at position 12 in KRAS as opposed to the activity of a different (i.e. another) oncogenic mutant form of KRAS that has a different oncogenic mutant residue (e.g. a different mutant amino acid residue as described elsewhere herein) at position 13 in KRAS. By way of example, in some embodiments, antibodies of the invention may preferentially inhibit activity of an oncogenic mutant form of KRAS having a D (aspartic acid) residue at 12 as opposed to the activity of an oncogenic mutant form of KRAS that has a different (i.e. non-D) oncogenic mutant residue (e.g. C) at position 13 in KRAS. The preferential inhibition may be an at least 5% higher, but typically at least 10% higher, preferably at least 20% higher, at least 30% higher, at least 40% higher, at least 50% higher, at least 60% higher, at least 70% higher, at least 80% higher, at least 90%, at least 100%, at least, 150% or at least 200% higher level (or amount) of inhibition.

In some embodiments, antibodies of the invention may preferentially inhibit activity of a given oncogenic mutant form of KRAS in accordance with the invention having a given oncogenic mutant residue (e.g. a mutant residue described elsewhere herein) at position 13 in KRAS as opposed to the activity of a different (i.e. another) oncogenic mutant form of KRAS that has a different oncogenic mutant residue (e.g. a different mutant amino acid residue as described elsewhere herein) at position 12 in KRAS. By way of example, in some embodiments, antibodies of the invention may preferentially inhibit activity of an oncogenic mutant form of KRAS having a D (aspartic acid) residue at 13 as opposed to the activity of an oncogenic mutant form of KRAS that has a different (i.e. non-D) oncogenic mutant residue (e.g. V or C or A or S or R, preferably V or C) at position 12 in KRAS. The preferential inhibition may be an at least 5% higher, but typically at least 10% higher, preferably at least 20% higher, at least 30% higher, at least 40% higher, at least 50% higher, at least 60% higher, at least 70% higher, at least 80% higher, at least 90%, at least 100%, at least, 150% or at least 200% higher level (or amount) of inhibition.

Inhibition of activity may be as described elsewhere herein (e.g. may be characterised by causing or inducing or increasing apoptosis, which may be determined as described elsewhere herein).

In some embodiments, an antibody of the invention causes (or elicits) a level (or amount) of apoptosis (which as discussed elsewhere herein is indicative of KRAS inhibition) of cells (e.g. cancer cells or cancer cell lines) that express an oncogenic mutant form of KRAS that has a given G12 mutation (mutation at position 12) in accordance with the invention (e.g. an oncogenic mutant form of KRAS having one of the potential mutant residues set forth in the amino acid sequence of SEQ ID NO:13 or SEQ ID NO:15) that is higher (e.g. significantly higher) than the level (or amount) of apoptosis that said antibody causes (or elicits) in cells that express an oncogenic mutant form of KRAS having a different oncogenic mutant residue at G12 mutation (mutation at position 12) (e.g. having a different one of the potential mutant residues set forth in the amino acid sequence of SEQ ID NO:13 or SEQ ID NO:15). For example, an antibody of the invention may cause (or elicit) a level (or amount) of apoptosis of cells (e.g. cancer cells or cancer cell lines) that express an oncogenic mutant form of KRAS that has a G12D mutation (e.g. SEQ ID NO:8 or SEQ ID NO: 11) that is higher (e.g. significantly higher) than the level (or amount) of apoptosis that said antibody causes (or elicits) in cells that express an oncogenic mutant form of KRAS having a different oncogenic mutant residue at G12 (different, i.e. non-D, mutation at position 12, (e.g. having a different, i.e. non-D, one of the potential mutant residues set forth in the amino acid sequence of SEQ ID NO:13 or SEQ ID NO:15). For example, an antibody of the invention may cause (or elicit) a level (or amount) of apoptosis of cells (e.g. cancer cells or cancer cell lines) that express an oncogenic mutant form of KRAS that has a G12D mutation (e.g. SEQ ID NO:8 or SEQ ID NO: 11) that is higher (e.g. significantly higher) than the level (or amount) of apoptosis that said antibody causes (or elicits) in cells that express an oncogenic mutant form of KRAS having a G12V or G12C mutation.

By way of another example, in some embodiments, an antibody of the invention causes (or elicits) a level (or amount) of apoptosis (which as discussed elsewhere herein is indicative of KRAS inhibition) of cells (e.g. cancer cells or cancer cell lines) that express an oncogenic mutant form of KRAS that has a given G13 mutation (mutation at position 13) in accordance with the invention (e.g. an oncogenic mutant form of KRAS having one of the potential mutant residues set forth in the amino acid sequence of SEQ ID NO:14 or SEQ ID NO:16) that is higher (e.g. significantly higher) than the level (or amount) of apoptosis that said antibody causes (or elicits) in cells that express an oncogenic mutant form of KRAS having a different oncogenic mutant residue at G13 (mutation at position 13) (e.g. having a different one of the potential mutant residues set forth in the amino acid sequence of SEQ ID NO:14 or SEQ ID NO:16). For example, an antibody of the invention may cause (or elicit) a level (or amount) of apoptosis of cells (e.g. cancer cells or cancer cell lines) that express an oncogenic mutant form of KRAS that has a G13D mutation (e.g. SEQ ID NO:9 or SEQ ID NO:12) that is higher (e.g. significantly higher) than the level (or amount) of apoptosis that said antibody causes (or elicits) in cells that express an oncogenic mutant form of KRAS having a different oncogenic mutant residue at G13 mutation (different, i.e. non-D, mutation at position 13). For example, an antibody of the invention may cause (or elicit) a level (or amount) of apoptosis of cells (e.g. cancer cells or cancer cell lines) that express an oncogenic mutant form of KRAS that has a G13D mutation (e.g. SEQ ID NO:9 or SEQ ID NO:12) that is higher (e.g. significantly higher) than the level (or amount) of apoptosis that said antibody causes (or elicits) in cells that express an oncogenic mutant form of KRAS having a G13C mutation.

In some such embodiments, the higher level of apoptosis caused in cells is a level that is at least 5% higher, but typically at least 10% higher, preferably at least 20% higher, at least 30% higher, at least 40% higher, at least 50% higher, at least 60% higher, at least 70% higher, at least 80% higher, at least 90%, at least 100%, at least, 150% or at least 200% higher. In some embodiments, the higher level of apoptosis is a level that is up to 500% higher, up to 400% higher, up to 300% higher, up to 200% higher, up to 100% higher or up to 50% higher. In some embodiments, the higher level of apoptosis caused in cells is a level that is up to 5-500% higher, 5-400% higher, 5-300% higher, 5-200% higher, 5-100% higher, 5-50% higher, 5-20% higher, 10-500% higher, 10-400% higher, 10-300% higher, 10-200% higher, 10-100% higher, 10-50% higher, 10-20% higher, 20-500% higher, 20-400% higher, 20-300% higher, 20-200% higher, 20-100% higher, 20-50% higher, 50-500% higher, 50-400% higher, 50-300% higher, 50-200% higher, or 50-100% higher.

As discussed elsewhere herein, in some embodiments, a cancer cell line that expresses an oncogenic G12 mutant form of KRAS may be the cancer cell line SK-LU-1 (which expresses the G12D oncogenic mutant form of KRAS). In some embodiments, a cancer cell line that expresses an oncogenic G13 mutant form of KRAS may be the cancer cell line HCT116 (which expresses the G13D oncogenic mutant form of KRAS). In some embodiments, a cancer cell line that expresses an oncogenic G12 mutant form of KRAS may be the cancer cell line SW620 (which expresses the G12V oncogenic mutant form of KRAS). In some embodiments, a cancer cell line that expresses an oncogenic G12 mutant form of KRAS may be the cancer cell line MIA-PA-CA-2 (which expresses the G12C oncogenic mutant form of KRAS).

Without wishing to be bound by theory, it is believed that antibodies of the present invention are able to inhibit activity of one or more oncogenic mutant forms of KRAS in accordance with the invention in cells after being internalized into cells by a process of macropinocytosis. Alternatively viewed, without wishing to be bound by theory, it believed that antibodies of the present invention are able to be internalized into cells expressing an oncogenic mutant form of KRAS in accordance with the invention by a process of macropinocytosis.

In another aspect, the present invention provides an antibody, for example an isolated antibody, which binds to an oncogenic mutant form of KRAS that has an amino acid sequence selected from the group consisting of SEQ ID NO:13, 14, 15, 16, 8, 9, 11 and 12, wherein said antibody binds to an epitope in the region of said oncogenic mutant form of KRAS that is defined by amino acid residues 10-21 of 13, 14, 15, 16, 8, 9, 11 or 12, and wherein said antibody inhibits activity of said oncogenic mutant form of KRAS. Discussion of various features of the antibodies of other aspects of the invention and preferred embodiments apply mutatis mutandis to this aspect of the invention.

In another aspect, the present invention provides an antibody, for example an isolated antibody, which binds to an oncogenic mutant form of KRAS that has an amino acid sequence selected from the group consisting of SEQ ID NO:8, 9, 11 and 12, wherein said antibody binds to an epitope in the region of said oncogenic mutant form of KRAS that is defined by amino acid residues 10-21 of SEQ ID NO:8, 9, 11 or 12, and wherein said antibody inhibits activity of said oncogenic mutant form of KRAS. Discussion of various features of the antibodies of other aspects of the invention and preferred embodiments apply mutatis mutandis to this aspect of the invention.

In another aspect, the present invention provides an antibody, for example an isolated antibody, which binds to an oncogenic mutant form of KRAS that has an amino acid sequence selected from the group consisting of SEQ ID NO:8 and SEQ ID NO:11, wherein said antibody binds to an epitope in the region of said oncogenic mutant form of KRAS that is defined by amino acid residues 10-21 of SEQ ID NO:8 or SEQ ID NO:11, and wherein said antibody inhibits activity of said oncogenic mutant form of KRAS. Discussion of various features of the antibodies of other aspects of the invention and preferred embodiments apply mutatis mutandis to this aspect of the invention.

In another aspect, the present invention provides an antibody, for example an isolated antibody, which binds to an oncogenic mutant form of KRAS that has an amino acid sequence selected from the group consisting of SEQ ID NO:9 and SEQ ID NO:12, wherein said antibody binds to an epitope in the region of said oncogenic mutant form of KRAS that is defined by amino acid residues 10-21 of SEQ ID NO:9 or SEQ ID NO:12, and wherein said antibody inhibits activity of said oncogenic mutant form of KRAS. Discussion of various features of the antibodies of other aspects of the invention and preferred embodiments apply mutatis mutandis to this aspect of the invention.

In another aspect, the present invention provides an antibody which binds to an oncogenic mutant form of KRAS, said oncogenic mutant form of KRAS comprising an amino acid substitution at the position corresponding to position G12 or G13 of wild-type KRAS (e.g. SEQ ID NO:7 or SEQ ID NO:10), wherein said antibody binds to an epitope that is in the region of said oncogenic mutant form of KRAS that is defined by the amino acid residues that correspond to amino acid residues 10-21 of wild-type KRAS (e.g. SEQ ID NO:7 or SEQ ID NO:10). Discussion of various features of the antibodies of other aspects of the invention and preferred embodiments apply mutatis mutandis to this aspect of the invention.

In another aspect, the present invention provides an antibody which binds to an oncogenic mutant form of KRAS that comprises an amino acid sequence selected from the group consisting of SEQ ID NO:17, 18, 1 and 2, wherein said antibody binds to an epitope of said oncogenic mutant form of KRAS in the region defined by the amino acid sequence of SEQ ID NO:17, 18, 1 or 2, and wherein said antibody inhibits activity of said oncogenic mutant form of KRAS. Discussion of various features of the antibodies of other aspects of the invention and preferred embodiments apply mutatis mutandis to this aspect of the invention.

In another aspect, the present invention provides an antibody which binds to an oncogenic mutant form of KRAS that comprises an amino acid sequence selected from the group consisting of SEQ ID NO:1 and 2, wherein said antibody binds to an epitope of said oncogenic mutant form of KRAS in the region defined by the amino acid sequence of SEQ ID NO:1 or 2, and wherein said antibody inhibits activity of said oncogenic mutant form of KRAS. Discussion of various features of the antibodies of other aspects of the invention and preferred embodiments apply mutatis mutandis to this aspect of the invention.

In another aspect, the present invention provides an antibody, for example an isolated antibody, which binds to an oncogenic mutant form of KRAS that has an amino acid sequence of selected from the group consisting of SEQ ID NO:13, 14, 15, 16, 8, 9, 11 and 12, wherein said antibody binds in the region of said oncogenic mutant form of KRAS that is defined by amino acid residues 10-21 of 13, 14, 15, 16, 8, 9, 11 or 12, and wherein said antibody inhibits activity of said oncogenic mutant form of KRAS. Discussion of various features of the antibodies of other aspects of the invention and preferred embodiments apply mutatis mutandis to this aspect of the invention.

In another aspect, the present invention provides an antibody, for example an isolated antibody, which binds to an oncogenic mutant form of KRAS (e.g. SEQ ID NO:13, 14, 15, 16, 8, 9, 11 and 12) at an epitope that comprises an amino acid substitution at the position corresponding to position G12 or G13 of wild-type KRAS (preferably a G12D or G13D substitution). Preferably, said antibody inhibits activity of said oncogenic mutant form of KRAS. Discussion of various features of the antibodies of other aspects of the invention and preferred embodiments apply mutatis mutandis to this aspect of the invention.

In another aspect, the present invention provides an antibody which binds to an oncogenic mutant form of KRAS, said oncogenic mutant form of KRAS comprising an amino acid substitution at the position corresponding to position G12 or G13 of wild-type KRAS (SEQ ID NO:7), wherein said antibody binds to an epitope that is in the region of said oncogenic mutant form of KRAS that is defined by the amino acid residues that correspond to amino acid residues 10-21 of wild-type KRAS (SEQ ID NO:7), and wherein said antibody inhibits (or is capable of inhibiting) GTPase activity of said oncogenic mutant form of KRAS. Discussion of various features of the antibodies of other aspects of the invention and preferred embodiments apply mutatis mutandis to this aspect of the invention.

In another aspect, the present invention provides an antibody which binds to an oncogenic mutant form of KRAS, said oncogenic mutant form of KRAS comprising an amino acid substitution at the position corresponding to position G12 or G13 of wild-type KRAS (SEQ ID NO:7), wherein said antibody binds to an epitope that is in the region of said oncogenic mutant form of KRAS that is defined by the amino acid residues that correspond to amino acid residues 10-21 of wild-type KRAS (SEQ ID NO:7), and wherein said antibody inhibits (or is capable of inhibiting) ERK phosphorylation in cells that express said oncogenic mutant form of KRAS. Discussion of various features of the antibodies of other aspects of the invention and preferred embodiments apply mutatis mutandis to this aspect of the invention.

In another aspect, the present invention provides an antibody which binds to an oncogenic mutant form of KRAS, said oncogenic mutant form of KRAS comprising an amino acid substitution at the position corresponding to position G12 or G13 of wild-type KRAS (SEQ ID NO:7), wherein said antibody binds to an epitope that is in the region of said oncogenic mutant form of KRAS that is defined by the amino acid residues that correspond to amino acid residues 10-21 of wild-type KRAS (SEQ ID NO:7), and wherein said antibody induces or increases (or is capable of inducing or increasing) apoptosis in cells that express said oncogenic mutant form of KRAS. Discussion of various features of the antibodies of other aspects of the invention and preferred embodiments apply mutatis mutandis to this aspect of the invention.

In another aspect, the present invention provides an antibody which binds to an oncogenic mutant form of KRAS, said oncogenic mutant form of KRAS comprising an amino acid substitution at the position corresponding to position G12 or G13 of wild-type KRAS (SEQ ID NO:7), wherein said antibody binds to an epitope that is in the region of said oncogenic mutant form of KRAS that is defined by the amino acid residues that correspond to amino acid residues 10-21 of wild-type KRAS (SEQ ID NO:7), and wherein said antibody induces or increases (or is capable of inducing or increasing) necrosis in cells that express said oncogenic mutant form of KRAS. Discussion of various features of the antibodies of other aspects of the invention and preferred embodiments apply mutatis mutandis to this aspect of the invention.

In another aspect, the present invention provides an antibody which preferentially (or selectively) binds to an oncogenic mutant form of KRAS as compared to wild-type KRAS, wherein said oncogenic mutant form of KRAS comprises an amino acid substitution at the position corresponding to position G12 or G13 of wild-type KRAS (SEQ ID NO:7), wherein said antibody binds to an epitope that is in the region of said oncogenic mutant form of KRAS that is defined by the amino acid residues that correspond to amino acid residues 10-21 of wild-type KRAS (SEQ ID NO:7), and wherein said antibody inhibits activity of said oncogenic mutant form of KRAS. Discussion of various features of the antibodies of other aspects of the invention and preferred embodiments apply mutatis mutandis to this aspect of the invention.

In another aspect, the present invention provides an antibody which preferentially (or selectively) binds to an oncogenic mutant form of KRAS as compared to wild-type KRAS, wherein said oncogenic mutant form of KRAS has an amino acid sequence of SEQ ID NO:9 or SEQ ID NO:12, wherein said antibody binds to an epitope that is in the region of said oncogenic mutant form of KRAS that is defined by the amino acid residues 10-21 of SEQ ID NO:9 or SEQ ID NO:12, and wherein said antibody inhibits activity of said oncogenic mutant form of KRAS. Discussion of various features of the antibodies of other aspects of the invention and preferred embodiments apply mutatis mutandis to this aspect of the invention.

In another aspect, the present invention provides an antibody which binds to an oncogenic mutant form of KRAS, wherein said oncogenic mutant form of KRAS comprises an amino acid substitution at the position corresponding to position G12 or G13 of wild-type KRAS (SEQ ID NO:7), wherein said antibody binds to an epitope that is in the region of said oncogenic mutant form of KRAS that is defined by the amino acid residues that correspond to amino acid residues 10-21 of wild-type KRAS (SEQ ID NO:7), and wherein said antibody preferentially (or selectively) inhibits activity of said oncogenic mutant form of KRAS as compared to wild-type KRAS. Discussion of various features of the antibodies of other aspects of the invention and preferred embodiments apply mutatis mutandis to this aspect of the invention.

In another aspect, the present invention provides an antibody which binds to an oncogenic mutant form of KRAS having an amino acid sequence of SEQ ID NO:9 or SEQ ID NO:12, wherein said antibody binds to an epitope that is in the region of said oncogenic mutant form of KRAS that is defined by amino acid residues 10-21 of SEQ ID NO:9 or SEQ ID NO:12, and wherein said antibody preferentially (or selectively) inhibits activity of said oncogenic mutant form of KRAS as compared to wild-type KRAS. Discussion of various features of the antibodies of other aspects of the invention and preferred embodiments apply mutatis mutandis to this aspect of the invention.

In another aspect, the present invention provides an antibody (e.g. a monoclonal antibody), for example an isolated antibody, which binds to (or is capable of binding to) an oncogenic mutant form of KRAS that has an amino acid sequence selected from the group consisting of SEQ ID NO:9 and SEQ ID NO:12, wherein said antibody binds to an epitope in the region of said oncogenic mutant form of KRAS that is defined by amino acid residues 10-21 of SEQ ID NO:9 or SEQ ID NO:12, and wherein said antibody has CDR domain amino acid sequences (or combinations thereof) and/or a at least one VH domain amino acid sequence and/or at least one VL domain amino acid sequence (or combinations of VH and VL domain sequences) as described elsewhere herein in connection with other aspects of the invention. For example, in some such embodiments, the antibody may have CDR domain amino acid sequences (or combinations thereof) and/or at least one VH domain amino acid sequence and/or at least one VL domain amino acid sequence (or combinations of VH and VL domain sequences) as described elsewhere herein in connection with the 11D6-1, 4B8-1, 7D11-1, 7G2-1, 6B2-1-1 or 6B2-1-2 antibodies (or sequences substantially homologous thereto, or sequences based on the sequences the 11D6-1, 4B8-1, 7D11-1, 7G2-1, 6B2-1-1 or 6B2-1-2 antibodies). In some embodiments, the antibody preferably inhibits activity of said oncogenic mutant form of KRAS (e.g. one or more activity as described elsewhere herein). In some embodiments, the antibody preferentially (or selectively) binds to said oncogenic mutant form of KRAS (e.g. as described elsewhere herein) and/or preferentially (or selectively) inhibits said oncogenic mutant form of KRAS (e.g. as described elsewhere herein). In some embodiments, the antibody preferentially (or selectively) binds to said oncogenic mutant form of KRAS as compared to wild-type KRAS and/or preferentially (or selectively) inhibits said oncogenic mutant form of KRAS as compared to wild-type KRAS. Discussion of various features of the antibodies of other aspects of the invention and preferred embodiments apply mutatis mutandis to this aspect of the invention.

In another aspect, the present invention provides an antibody (e.g. a monoclonal antibody), for example an isolated antibody, which binds to (or is capable of binding to) an isolated peptide that has the amino acid sequence of SEQ ID NO:5, and wherein said antibody has CDR domain amino acid sequences (or combinations thereof) and/or a at least one VH domain amino acid sequence and/or at least one VL domain amino acid sequence (or combinations of VH and VL domain sequences) as described elsewhere herein in connection with other aspects of the invention. For example, in some such embodiments, the antibody may have CDR domain amino acid sequences (or combinations thereof) and/or at least one VH domain amino acid sequence and/or at least one VL domain amino acid sequence (or combinations of VH and VL domain sequences) as described elsewhere herein in connection with the 11D6-1, 4B8-1, 7D11-1, 7G2-1, 6B2-1-1 or 6B2-1-2 antibodies (or sequences substantially homologous thereto, or sequences based on the sequences the 11D6-1, 4B8-1, 7D11-1, 7G2-1, 6B2-1-1 or 6B2-1-2 antibodies). In some embodiments, the antibody preferentially (or selectively) binds to an isolated peptide of SEQ ID NO:5 as compared to an isolated peptide of SEQ ID NO:160. In preferred embodiments of this aspect of the invention, the antibody binds (or is capable of binding to) an oncogenic mutant form of KRAS that has an amino acid sequence selected from the group consisting of SEQ ID NO:9 and SEQ ID NO:12, wherein said antibody binds to an epitope in the region of said oncogenic mutant form of KRAS that is defined by amino acid residues 10-21 of SEQ ID NO:9 or SEQ ID NO:12. In some embodiments, the antibody preferably inhibits activity of said oncogenic mutant form of KRAS (e.g. one or more activity as described elsewhere herein). In some embodiments, the antibody preferentially (or selectively) binds to said oncogenic mutant form of KRAS (e.g. as described elsewhere herein) and/or preferentially (or selectively) inhibits said oncogenic mutant form of KRAS (e.g. as described elsewhere herein). In some embodiments, the antibody preferentially (or selectively) binds to said oncogenic mutant form of KRAS as compared to wild-type KRAS and/or preferentially (or selectively) inhibits said oncogenic mutant form of KRAS as compared to wild-type KRAS. Discussion of various features of the antibodies of other aspects of the invention and preferred embodiments apply mutatis mutandis to this aspect of the invention.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, an “antibody”, as used herein, means “at least a first antibody”. The operable limits and parameters of combinations, as with the amounts of any single agent, will be known to those of ordinary skill in the art in light of the present disclosure.

In addition, where the terms “comprise”, “comprises”, “has” or “having”, or other equivalent terms are used herein, then in some more specific embodiments these terms include the term “consists of” or “consists essentially of”, or other equivalent terms.

Nucleic acid molecules comprising nucleotide sequences that encode the antibodies of the present invention as defined herein or parts or fragments thereof, or nucleic acid molecules substantially homologous thereto, form yet further aspects of the invention.

Preferred nucleic acid molecules are those encoding a VH region of an antibody of the present invention (e.g., those encoding SEQ ID NOs:30 or 50 or 70 or 90 or 110 or 130, such as SEQ ID NOs:28 or 48 or 68 or 88 or 108 or 128, respectively). Other preferred nucleic acid molecules are those encoding a VL region of an antibody of the present invention (e.g., those encoding SEQ ID NOs:31 or 51 or 71 or 91 or 111 or 131, such as SEQ ID NOs:29 or 49 or 69 or 89 or 109 or 129, respectively).

Thus, preferred nucleic acid molecules comprise sequences which encode a heavy chain variable region (VH) that has the amino acid sequence of SEQ ID NO: or 50 or 70 or 90 or 110 or 130 (which is preferably encoded by 28 or 48 or 68 or 88 or 108 or 128, respectively) and/or comprise sequences which encode a light chain variable region (VL) which has the amino acid sequence of SEQ ID NO: 31 or 51 or 71 or 91 or 111 or 131 (which is preferably encoded by SEQ ID NO: 29 or 49 or 69 or 89 or 109 or 129, respectively).

Also preferred are nucleic acids which encode the following combinations: SEQ ID NOs: 30 and 31; or SEQ ID NOs: 50 and 51; or SEQ ID NOs 70 and 71 or SEQ ID NOs: 90 and 91; or SEQ ID NOs: 110 and 111; or SEQ ID NOs: 130 and 131. Also preferred are nucleic acid molecules which comprise the following combinations: SEQ ID NOs: 28 and 29; or SEQ ID NOs: 48 and 49; or SEQ ID NOs: 68 and 69; or SEQ ID NOs: 88 and 89; or SEQ ID NOs: 108 and 109; or SEQ ID NOs: 128 and 129.

Other preferred nucleic acid molecules comprise sequences that encode IgG forms of the antibodies of the invention.

In another aspect, the present invention provides a set (or plurality) of nucleic acid molecules each comprising a nucleotide sequence, wherein said set of nucleic acid molecules together (or collectively) encode an antibody in accordance with the invention. Such a set of nucleic acid molecules may be characterised in that when the set is expressed (i.e. expressed together) (e.g. in a host cell) an entire antibody of the present invention is expressed and preferably assembled.

Nucleic acid sequences that a substantially homologous to the sequences described above may also be used.

The term “substantially homologous” as used herein in connection with an amino acid or nucleic acid sequence includes sequences having at least 65%, 70% or 75%, preferably at least 80%, and even more preferably at least 85%, 90%, 95%, 96%, 97%, 98% or 99%, sequence identity to the amino acid or nucleic acid sequence disclosed. Substantially homologous sequences of the invention thus include single or multiple base or amino acid alterations (additions, substitutions, insertions or deletions) to the sequences of the invention. At the amino acid level preferred substantially homologous sequences contain up to 5, e.g. only 1, 2, 3, 4 or 5, preferably 1, 2 or 3, more preferably 1 or 2, altered amino acids, in one or more of the framework regions and/or one or more of the CDRs making up the sequences of the invention. Said alterations can be with conservative or non-conservative amino acids. Preferably said alterations are conservative amino acid substitutions.

In certain embodiments, if a given starting sequence is relatively short (e.g. five amino acids in length), then fewer amino acid substitutions may be present in sequences substantially homologous thereto as compared with the number of amino acid substitutions that might optionally be made in a sequence substantially homologous to a longer starting sequence. For example, in certain embodiments, a sequence substantially homologous to a starting VH CDR1 sequence in accordance with the present invention, e.g. a starting VH CDR1 sequence which in some embodiments may be five amino acid residues in length, preferably has 1 or 2 (more preferably 1) altered amino acids in comparison with the starting sequence. Accordingly, in some embodiments the number of altered amino acids in substantially homologous sequences (e.g. in substantially homologous CDR sequences) can be tailored to the length of a given starting CDR sequence. For example, different numbers of altered amino acids can be present depending on the length of a given starting CDR sequence such as to achieve a particular % sequence identity in the CDRs, for example a sequence identity of at least 75%, 80%, 85%, 90% or 95%.

Routine methods in the art such as alanine scanning mutagenesis and/or analysis of crystal structure of the antigen-antibody complex can be used in order to determine which amino acid residues of the CDRs do not contribute or do not contribute significantly to antigen binding and therefore are good candidates for alteration or substitution in the embodiments of the invention involving substantially homologous sequences.

The term “substantially homologous” also includes modifications or chemical equivalents of the amino acid and nucleotide sequences of the present invention that perform substantially the same function as the proteins or nucleic acid molecules of the invention in substantially the same way. For example, any substantially homologous antibody should retain the ability to bind to a given oncogenic mutant form of KRAS in accordance with the invention (e.g. as described elsewhere herein). Preferably, any substantially homologous antibody should retain one or more (or all) of the functional capabilities of the starting antibody.

Substantially homologous sequences of antibodies of the invention also include, without limitation, for example alterations that do not affect the VH, VL or CDR domains of the antibodies, e.g. antibodies where tag sequences or other components are added that do not contribute to the binding of antigen, or alterations to convert one type or format of antibody molecule or fragment to another type or format of antibody molecule or fragment (e.g. conversion from Fab to scFv or whole antibody or vice versa), or the conversion of an antibody molecule to a particular class or subclass of antibody molecule (e.g. the conversion of an antibody molecule to IgG or a subclass thereof, e.g. IgG₂ or IgG₄).

Preferably, any substantially homologous antibody should retain the ability to bind (or specifically bind) to the same (or substantially the same) epitope of a given oncogenic mutant form of KRAS as recognized by the antibody in question, for example, the same epitope recognized by the CDR domains of the invention or the VH and VL domains of the invention as described herein. Thus, preferably, any substantially homologous antibody should retain the ability to compete with one or more of the various antibodies of the invention (e.g. one or more of the described polyclonal antibodies or one or more of the described monoclonal antibodies 11D6-1, 4B8-1, 7D11-1, 7G2-1, 6B2-1-1 or 6B2-1-2) for binding to a given oncogenic mutant form of KRAS. Binding to the same epitope/antigen can be readily tested by methods well known and described in the art, e.g. using binding assays, e.g. a competition assay. Retention of other functional properties can also readily be tested by methods well known and described in the art or herein.

Thus, a person skilled in the art will appreciate that binding assays can be used to test whether “substantially homologous” antibodies have the same binding specificities as the antibodies and antibody fragments of the invention, for example, binding assays such as competition assays or ELISA assays as described elsewhere herein. BIAcore assays could also readily be used to establish whether “substantially homologous” antibodies can bind to a given oncogenic mutant form of KRAS. The skilled person will be aware of other suitable methods and variations.

As outlined below, a competition binding assay can be used to test whether “substantially homologous” antibodies retain the ability to bind (or specifically bind) to substantially the same epitope (or the same epitope) of an oncogenic mutant form of KRAS as recognized by the antibodies of the invention (e.g. antibodies 11D6-1, 4B8-1, 7D11-1, 7G2-1, 6B2-1-1 or 6B2-1-2, or antibodies based on these antibodies), or have the ability to compete with one or more of the various antibodies of the invention (e.g. antibodies 11D6-1, 4B8-1, 7D11-1, 7G2-1, 6B2-1-1 or 6B2-1-2, or antibodies based on these antibodies). The method described below is only one example of a suitable competition assay. The skilled person will be aware of other suitable methods and variations.

An exemplary competition assay involves assessing the binding of various effective concentrations of an antibody of the invention to a given oncogenic mutant form of KRAS in the presence of varying concentrations of a test antibody (e.g. a substantially homologous antibody). The amount of inhibition of binding induced by the test antibody can then be assessed. A test antibody that shows increased competition with an antibody of the invention at increasing concentrations (i.e. increasing concentrations of the test antibody result in a corresponding reduction in the amount of antibody of the invention binding to an oncogenic mutant form of KRAS) is evidence of binding to substantially the same epitope. Preferably, the test antibody significantly reduces the amount of antibody of the invention that binds to an oncogenic mutant form of KRAS. Preferably, the test antibody reduces the amount of antibody of the invention that binds to an oncogenic mutant form of KRAS by at least about 60%, 65%, 70%, 75%, 80%, 85%, 90% or 95%. ELISA assays may be used for assessing inhibition of binding in such a competition assay but other suitable techniques would be well known to a person skilled in the art.

In some embodiments, “substantially homologous” antibodies which retain the ability to bind (or specifically bind) to substantially the same (or the same) epitope of a given oncogenic form of KRAS as recognized by antibodies of the invention (e.g. antibodies 11D6-1, 4B8-1, 7D11-1, 7G2-1, 6B2-1-1 or 6B2-1-2, or antibodies based on these antibodies) or which have the ability to compete with one or more of the various antibodies of the invention (e.g. antibodies 11D6-1, 4B8-1, 7D11-1, 7G2-1, 6B2-1-1 or 6B2-1-2, or antibodies based on these antibodies) are preferred.

The term “competing antibodies”, as used herein, refers to antibodies that bind to about, substantially or essentially the same, or even the same, epitope as a “reference antibody”. “Competing antibodies” include antibodies with overlapping epitope specificities. Competing antibodies are thus able to effectively compete with a reference antibody for binding to an oncogenic mutant form of KRAS in accordance with the invention. Preferably, the competing antibody can bind to the same epitope as the reference antibody. Alternatively viewed, the competing antibody preferably has the same epitope specificity as the reference antibody.

“Reference antibodies” as used herein include antibodies (e.g. the polyclonal rabbit antibodies or monoclonal antibodies described herein) that bind to an isolated peptide of the invention or to an epitope of an oncogenic mutant form of KRAS in accordance with the invention. “Reference antibodies” also include antibodies which preferably have a VH and a VL domain as defined herein, more preferably a VH domain of SEQ ID NO: 30 and a VL domain of SEQ ID NO: 31, or a VH domain of SEQ ID NO: 50 and a VL domain of SEQ ID NO: 51, a VH domain of SEQ ID NO: 70 and a VL domain of SEQ ID NO: 71, a VH domain of SEQ ID NO: 90 and a VL domain of SEQ ID NO: 91, a VH domain of SEQ ID NO: 110 and a VL domain of SEQ ID NO: 111, or a VH domain of SEQ ID NO: 130 and a VL domain of SEQ ID NO: 131. Certain preferred reference antibodies are selected from antibodies 11D6-1, 4B8-1, 7D11-1, 7G2-1, 6B2-1-1 or 6B2-1-2, or antibodies based on these antibodies.

As the identification of competing antibodies is determined in comparison to a reference antibody, it will be understood that actually determining the epitope to which either or both antibodies bind is not in any way required in order to identify a competing antibody. However, epitope mapping can be performed using standard techniques, if desired.

In the following descriptions of the compositions, immunoconjugates, pharmaceuticals, combinations, cocktails, kits, first and second medical uses and all methods in accordance with this invention, the terms “antibody” and “immunoconjugate”, or an antigen-binding region or fragment thereof, unless otherwise specifically stated or made clear from the scientific terminology, refer to a range of antibodies that bind to an oncogenic mutant form of KRAS in accordance with the invention as well as to the specific antibodies described in the Example section herein.

The terms “antibody” and “immunoglobulin”, as used herein, refer broadly to any immunological binding agent that comprises an antigen binding domain, including polyclonal and monoclonal antibodies.

Thus, the term “antibody” includes immunological binding agents that comprise an antigen binding domain obtained from or derived from an antibody (or based on an antigen binding domain of an antibody), e.g. obtained from or derived from an Ig (e.g. IgG) antibody (or based on an antigen binding domain of an Ig (e.g. IgG) antibody).

In some embodiments, polyclonal antibodies are preferred (e.g. polyclonal antibodies that are generated in (or raised in or isolated from) an animal (e.g. a rabbit such as a specific pathogen free (SPF) rabbit) immunized with an isolated peptide or conjugate (preferably a conjugate) of the present invention. Preferred isolated peptides and conjugates are described elsewhere herein.

In some embodiments, monoclonal antibodies are preferred (e.g. mouse monoclonal or human monoclonal antibodies or humanized monoclonal antibodies or rabbit monoclonal antibodies).

Depending on the type of constant domain in the heavy chains, whole antibodies are assigned to one of five major classes: IgA, IgD, IgE, IgG, and IgM and the antibodies of the invention may be in any one of these classes. Several of these are further divided into subclasses or isotypes, such as IgG1, IgG2, IgG3, IgG4, and the like. The heavy-chain constant domains that correspond to the difference classes of immunoglobulins are termed α, δ, ε, γ and μ respectively. The subunit structures and three-dimensional configurations of different classes of immunoglobulins are well known.

Generally, where whole antibodies rather than antigen binding regions are used in the invention, IgG (e.g. IgG₂ or IgG₄) and/or IgM are preferred because they are the most common antibodies in the physiological situation and because they are most easily made in a laboratory setting.

The “light chains” of mammalian antibodies are assigned to one of two clearly distinct types: kappa (κ) and lambda (λ), based on the amino acid sequences of their constant domains and some amino acids in the framework regions of their variable domains.

As will be understood by those in the art, the immunological binding reagents encompassed by the term “antibody” includes or extends to all antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any antibody-like molecule that has an antigen binding region, and this term includes antibody fragments that comprise an antigen binding domain such as Fab′, Fab, F(ab′)₂, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like.

The techniques for preparing and using various antibody-based constructs and fragments are well known in the art. Diabodies, in particular, are further described in EP 404 097 and WO 93/11161; whereas linear antibodies are further described in the art.

In some embodiments, the antibodies of the invention are non-human antibodies (e.g. rabbit or rat or mouse antibodies). In some embodiments, the antibodies of the invention are rabbit antibodies. In some embodiments, the antibodies are mouse antibodies.

In some embodiments, the antibodies of the invention are human antibodies, more preferably fully human antibodies. In this regard, human antibodies generally have at least two potential advantages for use in human therapy. First, the human immune system should not recognize the antibody as foreign. Second, the half-life in the human circulation will be similar to naturally occurring human antibodies, allowing smaller and less frequent doses to be given.

The term “human” as used herein in connection with antibody molecules and binding proteins first refers to antibodies and binding proteins having variable regions (e.g., V_{H}, V_{L}, CDR or FR regions) and, optionally, constant antibody regions, isolated or derived from a human repertoire or derived from or corresponding to sequences found in humans or a human repertoire, e.g., in the human germline or somatic cells.

“Human” antibodies and binding proteins further include amino acid residues not encoded by human sequences, e.g., mutations introduced by random or site directed mutations in vitro, for example mutations introduced by in vitro cloning or PCR. Particular examples of such mutations are mutations that involve conservative substitutions or other mutations in a small number of residues of the antibody or binding protein, e.g., in up to 5, 4, 3, 2 or 1 of the residues of the antibody or binding protein, preferably e.g., in up to 5, 4, 3, 2 or 1 of the residues making up one or more of the CDRs of the antibody or binding protein. Certain examples of such “human” antibodies include antibodies and variable regions that have been subjected to standard modification techniques to reduce the amount of potentially immunogenic sites.

Thus, “human” antibodies include sequences derived from and related to sequences found in humans, but which may not naturally exist within the human antibody germline repertoire in vivo. In addition, human antibodies and binding proteins include proteins comprising human consensus sequences identified from human sequences, or sequences substantially homologous to human sequences.

In addition, human antibodies and binding proteins are not limited to combinations of V_{H}, V_{L}, CDR or FR regions that are themselves found in combination in human antibody molecules. Thus, human antibodies and binding proteins can include or correspond to combinations of such regions that do not necessarily exist naturally in humans (e.g. are not naturally occurring antibodies).

In some embodiments, human antibodies will be fully human antibodies. “Fully human” antibodies, as used herein, are antibodies comprising “human” variable region domains and/or CDRs, without substantial non-human antibody sequences or without any non-human antibody sequences. For example, antibodies comprising human variable region domains and/or CDRs “without substantial non-human antibody sequences” are antibodies, domains and/or CDRs in which only up to 5, 4, 3, 2 or 1 amino acids are amino acids that are not encoded by human antibody sequences. Thus, “fully human” antibodies are distinguished from “humanized” antibodies, which are based on substantially non-human variable region domains, e.g., mouse variable region domains, in which certain amino acids have been changed to better correspond with the amino acids typically present in human antibodies.

The “fully human” antibodies of the invention may be human variable region domains and/or CDRs without any other substantial antibody sequences, such as being single chain antibodies. Alternatively, the “fully human” antibodies of the invention may be human variable region domains and/or CDRs integral with or operatively attached to one or more human antibody constant regions. Certain preferred fully human antibodies are IgG antibodies with the full complement of IgG constant regions.

In other embodiments, “human” antibodies of the invention will be part-human chimeric antibodies. “Part-human chimeric” antibodies, as used herein, are antibodies comprising “human” variable region domains and/or CDRs operatively attached to, or grafted onto, a constant region of a non-human species, such as rat or mouse. Such part-human chimeric antibodies may be used, for example, in pre-clinical studies, wherein the constant region will preferably be of the same species of animal used in the pre-clinical testing. These part-human chimeric antibodies may also be used, for example, in ex vivo diagnostics, wherein the constant region of the non-human species may provide additional options for antibody detection.

In some embodiments, the antibodies of the invention will be humanized antibodies. “Humanized” antibodies, which are based on substantially non-human variable region domains are antibodies in which certain amino acids have been changed to better correspond with the amino acids typically present in human antibodies. Methods for generating humanized antibodies are well known in the art.

For example, humanized antibodies can be accomplished by inserting the appropriate CDRs (e.g. murine CDRs) into a human antibody “scaffold”. In some cases, one or more CDR residues may be changed to better correspond with the amino acids typically present in human antibodies.

The term “heavy chain complementarity determining region” (“heavy chain CDR”) as used herein refers to regions of hypervariability within the heavy chain variable region (V_{H} domain) of an antibody molecule. The heavy chain variable region has three CDRs termed heavy chain CDR1, heavy chain CDR2 and heavy chain CDR3 from the amino terminus to carboxy terminus. The heavy chain variable region also has four framework regions (FR1, FR2, FR3 and FR4 from the amino terminus to carboxy terminus). These framework regions separate the CDRs.

The term “heavy chain variable region” (V_{H} domain) as used herein refers to the variable region of a heavy chain of an antibody molecule.

The term “light chain complementarity determining region” (“light chain CDR”) as used herein refers to regions of hypervariability within the light chain variable region (V_{L} domain) of an antibody molecule. Light chain variable regions have three CDRs termed light chain CDR1, light chain CDR2 and light chain CDR3 from the amino terminus to the carboxy terminus. The light chain variable region also has four framework regions (FR1, FR2, FR3 and FR4 from the amino terminus to carboxy terminus). These framework regions separate the CDRs.

The term “light chain variable region” (V_{L} domain) as used herein refers to the variable region of a light chain of an antibody molecule.

The CDRs of the antibodies of the invention are preferably separated by appropriate framework regions such as those found in naturally occurring antibodies and/or effective engineered antibodies. Thus, the V_{H}, V_{L} and individual CDR sequences are preferably provided within or incorporated into an appropriate framework or scaffold to enable antigen binding. Such framework sequences or regions may correspond to naturally occurring framework regions, FR1, FR2, FR3 and/or FR4, as appropriate to form an appropriate scaffold, or may correspond to consensus framework regions, for example identified by comparing various naturally occurring framework regions. Alternatively, non-antibody scaffolds or frameworks, e.g. T cell receptor frameworks can be used. Appropriate sequences that can be used for framework regions are well known and documented in the art and any of these may be used. In particular, framework regions that allow the maintenance of antigen specificity, for example framework regions that result in substantially the same or the same 3D structure of the antibody.

CDR sequences of certain antibodies of the invention are set forth herein in Tables A-F. In some other embodiments, CDR sequences of antibodies of the invention may be CDR sequences in the VH domains and VL domains of antibodies of the invention as identified using any suitable method (or tool), for example as identified according to the well-known methods of Kabat (e.g. Kabat, et al., “Sequences of Proteins of Immunological Interest”, 5th Ed. Public Health Service, National Institutes of Health, Bethesda, Md., 647-669, 1991) or Chothia (e.g. Chothia C, et al. (1989) Nature, 342:877-883, or Al-Lazikani et al., (1997) JMB 273, 927-948).

Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

In certain embodiments, the antibody or antibody fragment of the present invention comprises all or a portion of a heavy chain constant region, such as an IgG1, IgG2, IgG3, IgG4, IgA1, IgA2, IgE, IgM or IgD constant region. Preferably, the heavy chain constant region is an IgG heavy chain constant region, e.g. an IgG2 or an IgG4 heavy chain constant region, or a portion thereof. Furthermore, the antibody or antibody fragment can comprise all or a portion of a kappa light chain constant region or a lambda light chain constant region, or a portion thereof. All or part of such constant regions may be produced naturally or may be wholly or partially synthetic. Appropriate sequences for such constant regions are well known and documented in the art. When a full complement of constant regions from the heavy and light chains are included in the antibodies of the invention, such antibodies are typically referred to herein as “full length” antibodies or “whole” antibodies. Thus, in some embodiments, the antibodies of the invention are Ig (e.g. IgG) antibodies.

Preferably, in some embodiments, antibodies of the present invention (e.g. Ig antibodies) comprise two identical VH domains and two identical VL domains. Thus, in the case of Ig (e.g. IgG) antibodies for example, Ig antibodies typically comprise (or consist of) two identical heavy chains and two identical light chains, which together form the Ig antibody. However, in some embodiments, antibodies of the present invention (e.g. IgG antibodies) may comprise two identical VH domains and two non-identical VL domains (i.e. two different VL domains). Thus, in some embodiments, each antibody (e.g. IgG) molecule may comprise two identical VH domains and two non-identical VL domains (i.e. two different VL domains each having a different a different amino acid sequence). For example, an Ig (e.g. IgG) antibody may comprise (or consist of) two identical heavy chains and two light chains wherein the two light chains are not identical in sequence to each other (i.e. they are different).

By way of example, in some embodiments, an antibody may comprise at least one (preferably two) heavy chain variable region (VH domain) comprising the amino acid sequences of the three VH CDRs of the 6B2-1-1 (or 6B2-1-2) antibody (or CDR sequences substantially homologous thereto) and at least one (preferably one) light chain variable region (VL domain) comprising the amino acid sequences of the three VL CDRs of the 6B2-1-1 antibody (or CDR sequences substantially homologous thereto) and at least one (preferably one) light chain variable region (VL domain) comprising the amino acid sequences of three VL CDRs of the 6B2-1-2 antibody CDR sequences substantially homologous thereto. Thus, in some embodiments, an antibody may comprise at least one (preferably two) heavy chain variable region (VH domain) comprising the amino acid sequence of SEQ ID NO:110 (or a sequence substantially homologous thereto) and at least one (preferably one) light chain variable region (VL domain) comprising the amino acid sequence of SEQ ID NO:111 (or a sequence substantially homologous thereto) and at least one (preferably one) light chain variable region (VL domain) comprising the amino acid sequence of SEQ ID NO:131 (or a sequence substantially homologous thereto). In some embodiments, such antibodies may be Ig (e.g. IgG) antibodies.

The antibodies or antibody fragments can be produced naturally or can be wholly or partially synthetically produced. Thus the antibody may be from any appropriate source, for example recombinant sources and/or produced in transgenic animals or transgenic plants, or in eggs using the IgY technology. Thus, the antibody molecules can be produced in vitro or in vivo.

Preferably, the antibody or antibody fragment comprises an antibody light chain variable region (V_{L}) that comprises three CDR domains and an antibody heavy chain variable region (V_{H}) that comprises three CDR domains. Said VL and VH generally form the antigen binding site.

An “Fv” fragment is the minimum antibody fragment that contains a complete antigen-recognition and binding site. This region has a dimer of one heavy chain and one light chain variable domain in tight, non-covalent association. It is in this configuration that the three hypervariable regions (CDRs) of each variable domain interact to define an antigen-binding site on the surface of the V_{H}-V_{L} dimer. Collectively, the six hypervariable regions (CDRs) confer antigen-binding specificity to the antibody.

However, it is well documented in the art that the presence of three CDRs from the light chain variable domain and three CDRs from the heavy chain variable domain of an antibody is not always necessary for antigen binding. Thus, constructs smaller than the above classical antibody fragment are known to be effective.

For example, camelid antibodies have an extensive antigen binding repertoire but are devoid of light chains. Also, results with single domain antibodies comprising VH domains alone or VL domains alone show that these domains can bind to antigen with acceptably high affinities. Thus, three CDRs can effectively bind antigen.

Thus, although preferred antibodies of the invention might comprise six CDR regions (three from a light chain and three from a heavy chain), antibodies with fewer than six CDR regions (e.g. 3 CDR regions) are encompassed by the invention. Antibodies with CDRs from only the heavy chain or light chain are also contemplated.

A yet further aspect of the invention provides an antibody, preferably an isolated antibody, which binds to an oncogenic mutant form of KRAS in accordance with the invention and which has the ability to compete with (i.e. bind to the same or substantially the same epitope as) an antibody of the invention for binding to said oncogenic mutant form of KRAS. For example, antibodies that can compete with antibodies (e.g. polyclonal antibodies such as those described in the Example section herein) that have been generated against isolated peptides (or conjugates) of the invention for binding to an oncogenic mutant form of KRAS in accordance with the invention represent a further aspect of the invention. Other features and properties of other aspects of the invention apply, mutatis mutandis, to this aspect of the invention.

Binding to the same epitope/antigen can be readily tested by methods well known and described in the art, e.g. using binding assays such as a competition assay.

An exemplary competition assay involves assessing the binding of various effective concentrations of an antibody of the invention to an oncogenic mutant form of KRAS in accordance with the invention (e.g. a rabbit polyclonal antibody that binds to an isolated peptide or epitope of an oncogenic mutant form of KRAS in accordance with the invention of the invention) in the presence of varying concentrations of a test antibody (e.g. a substantially homologous antibody). The amount of inhibition of binding induced by the test antibody can then be assessed. A test antibody that shows increased competition with an antibody of the invention at increasing concentrations (i.e. increasing concentrations of the test antibody result in a corresponding reduction in the amount of antibody of the invention binding to an oncogenic mutant form of KRAS in accordance with the invention) is evidence of binding to substantially the same epitope. Preferably, the test antibody significantly reduces the amount of antibody of the invention that binds to an oncogenic mutant form of KRAS in accordance with the invention. Preferably, the test antibody reduces the amount of antibody of the invention that binds to an oncogenic mutant form of KRAS in accordance with the invention by at least about 60%, 65%, 70%, 75%, 80%, 85% or 95%. ELISA may, for example, be used for assessing inhibition of binding in such a competition assay but other suitable techniques would be well known to a person skilled in the art.

The term “competing antibodies”, as used herein, refers to antibodies that bind to about, substantially or essentially the same, or even the same, epitope as a “reference antibody”. “Competing antibodies” include antibodies with overlapping epitope specificities. Competing antibodies are thus able to effectively compete with a reference antibody for binding to an oncogenic mutant form of KRAS in accordance with the invention. Preferably, the competing antibody can bind to the same epitope as the reference antibody. Alternatively viewed, the competing antibody preferably has the same epitope specificity as the reference antibody.

“Reference antibodies” as used herein are antibodies (e.g. the polyclonal rabbit antibodies described herein) that bind to an isolated peptide or epitope of an oncogenic mutant form of KRAS in accordance with the invention.

As the identification of competing antibodies is determined in comparison to a reference antibody, it will be understood that actually determining the epitope to which either or both antibodies bind is not in any way required in order to identify a competing antibody. However, epitope mapping can be performed using standard techniques, if desired.

In the following descriptions of the compositions, immunoconjugates, pharmaceuticals, combinations, cocktails, kits, first and second medical uses and all methods in accordance with this invention, the terms “antibody” and “immunoconjugate”, or an antigen-binding region or fragment thereof, unless otherwise specifically stated or made clear from the scientific terminology, refer to a range of antibodies that bind to an oncogenic mutant form of KRAS in accordance with the invention as well as to the specific antibodies described in the Example section herein.

Preferably, the above described abilities and properties are observed at a measurable or significant level and more preferably at a statistically significant level, when compared to appropriate control levels. Appropriate significance levels are discussed elsewhere herein. More preferably, one or more of the above described abilities and properties are observed at a level which is measurably better, or more preferably significantly better, when compared to the abilities observed for prior art antibodies.

In any statistical analysis referred to herein, preferably the statistically significant difference over a relevant control or other comparative entity or measurement has a probability value of <0.1, preferably <0.05 or <0.01 or <0.001 or <0.0001. Appropriate methods of determining statistical significance are well known and documented in the art and any of these may be used.

In another aspect, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:32 or a sequence substantially homologous thereto,

(b) a VH CDR2 that has the amino acid sequence of SEQ ID NO:33 or a sequence substantially homologous thereto, and

(c) a VH CDR3 that has the amino acid sequence of SEQ ID NO:34 or a sequence substantially homologous thereto; and/or (preferably “and”) wherein said light chain variable region comprises:

(d) a variable light (VL) CDR1 that has the amino acid sequence of SEQ ID NO:35 or a sequence substantially homologous thereto,

(e) a VL CDR2 that has the amino acid sequence of SEQ ID NO:36 or a sequence substantially homologous thereto, and

(f) a VL CDR3 that has the amino acid sequence of SEQ ID NO:37 or a sequence substantially homologous thereto;

wherein said substantially homologous sequence is a sequence containing 1, 2 or 3 amino acid substitutions compared to the given CDR sequence, or wherein said substantially homologous sequence is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: or a sequence substantially homologous thereto, and/or (preferably “and”) a VL domain that has the amino acid sequence of SEQ ID NO: 31 or a sequence substantially homologous thereto.

In another aspect, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:52 or a sequence substantially homologous thereto,

(b) a VH CDR2 that has the amino acid sequence of SEQ ID NO:53 or a sequence substantially homologous thereto, and

(c) a VH CDR3 that has the amino acid sequence of SEQ ID NO:54 or a sequence substantially homologous thereto; and/or (preferably “and”) wherein said light chain variable region comprises:

(d) a variable light (VL) CDR1 that has the amino acid sequence of SEQ ID NO:55 or a sequence substantially homologous thereto,

(e) a VL CDR2 that has the amino acid sequence of SEQ ID NO:56 or a sequence substantially homologous thereto, and

(f) a VL CDR3 that has the amino acid sequence of SEQ ID NO:57 or a sequence substantially homologous thereto;

wherein said substantially homologous sequence is a sequence containing 1, 2 or 3 amino acid substitutions compared to the given CDR sequence, or wherein said substantially homologous sequence is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 50 or a sequence substantially homologous thereto, and/or (preferably “and”) a VL domain that has the amino acid sequence of SEQ ID NO: 51 or a sequence substantially homologous thereto.

In another aspect, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:72 or a sequence substantially homologous thereto,

(b) a VH CDR2 that has the amino acid sequence of SEQ ID NO:73 or a sequence substantially homologous thereto, and

(c) a VH CDR3 that has the amino acid sequence of SEQ ID NO:74 or a sequence substantially homologous thereto; and/or (preferably “and”) wherein said light chain variable region comprises:

(d) a variable light (VL) CDR1 that has the amino acid sequence of SEQ ID NO:75 or a sequence substantially homologous thereto,

(e) a VL CDR2 that has the amino acid sequence of SEQ ID NO:76 or a sequence substantially homologous thereto, and

(f) a VL CDR3 that has the amino acid sequence of SEQ ID NO:77 or a sequence substantially homologous thereto;
wherein said substantially homologous sequence is a sequence containing 1, 2 or 3 amino acid substitutions compared to the given CDR sequence, or wherein said substantially homologous sequence is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 70 or a sequence substantially homologous thereto, and/or (preferably “and”) a VL domain that has the amino acid sequence of SEQ ID NO: 71 or a sequence substantially homologous thereto.

In another aspect, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:92 or a sequence substantially homologous thereto,

(b) a VH CDR2 that has the amino acid sequence of SEQ ID NO:93 or a sequence substantially homologous thereto, and

(c) a VH CDR3 that has the amino acid sequence of SEQ ID NO:94 or a sequence substantially homologous thereto; and/or (preferably “and”) wherein said light chain variable region comprises:

(d) a variable light (VL) CDR1 that has the amino acid sequence of SEQ ID NO:95 or a sequence substantially homologous thereto,

(e) a VL CDR2 that has the amino acid sequence of SEQ ID NO:96 or a sequence substantially homologous thereto, and

(f) a VL CDR3 that has the amino acid sequence of SEQ ID NO:97 or a sequence substantially homologous thereto;

wherein said substantially homologous sequence is a sequence containing 1, 2 or 3 amino acid substitutions compared to the given CDR sequence, or wherein said substantially homologous sequence is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 90 or a sequence substantially homologous thereto, and/or (preferably “and”) a VL domain that has the amino acid sequence of SEQ ID NO: 91 or a sequence substantially homologous thereto.

In another aspect, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO: 112 or a sequence substantially homologous thereto,

(b) a VH CDR2 that has the amino acid sequence of SEQ ID NO:113 or a sequence substantially homologous thereto, and

(c) a VH CDR3 that has the amino acid sequence of SEQ ID NO:114 or a sequence substantially homologous thereto; and/or (preferably “and”) wherein said light chain variable region comprises:

(d) a variable light (VL) CDR1 that has the amino acid sequence of SEQ ID NO:115 or a sequence substantially homologous thereto,

(e) a VL CDR2 that has the amino acid sequence of SEQ ID NO:116 or a sequence substantially homologous thereto, and

(f) a VL CDR3 that has the amino acid sequence of SEQ ID NO:117 or a sequence substantially homologous thereto;

wherein said substantially homologous sequence is a sequence containing 1, 2 or 3 amino acid substitutions compared to the given CDR sequence, or wherein said substantially homologous sequence is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 110 or a sequence substantially homologous thereto, and/or (preferably “and”) a VL domain that has the amino acid sequence of SEQ ID NO: 111 or a sequence substantially homologous thereto.

In another aspect, the present invention provides an antibody, for example an isolated antibody, that binds to an oncogenic mutant form of KRAS and that comprises at least one heavy chain variable region that comprises three CDRs and at least one light chain variable region that comprises three CDRs, wherein said heavy chain variable region comprises:

(a) a variable heavy (VH) CDR1 that has the amino acid sequence of SEQ ID NO:132 or a sequence substantially homologous thereto,

(b) a VH CDR2 that has the amino acid sequence of SEQ ID NO:133 or a sequence substantially homologous thereto, and

(c) a VH CDR3 that has the amino acid sequence of SEQ ID NO:134 or a sequence substantially homologous thereto; and/or (preferably “and”) wherein said light chain variable region comprises:

(d) a variable light (VL) CDR1 that has the amino acid sequence of SEQ ID NO:135 or a sequence substantially homologous thereto,

(e) a VL CDR2 that has the amino acid sequence of SEQ ID NO:136 or a sequence substantially homologous thereto, and

(f) a VL CDR3 that has the amino acid sequence of SEQ ID NO:137 or a sequence substantially homologous thereto;

wherein said substantially homologous sequence is a sequence containing 1, 2 or 3 amino acid substitutions compared to the given CDR sequence, or wherein said substantially homologous sequence is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 130 or a sequence substantially homologous thereto, and/or (preferably “and”) a VL domain that has the amino acid sequence of SEQ ID NO: 131 or a sequence substantially homologous thereto.

The antibodies, peptides, binding proteins and nucleic acid molecules of the invention are generally “isolated” or “purified” molecules insofar as they are distinguished from any such components that may be present in situ within a human or animal body or a tissue sample derived from a human or animal body. The sequences may, however, correspond to or be substantially homologous to sequences as found in a human or animal body. Thus, the term “isolated” or “purified” as used herein in reference to nucleic acid molecules or sequences and proteins, peptides or polypeptides, e.g. antibodies, refers to such molecules when isolated from, purified from, or substantially free of their natural environment, e.g. isolated from or purified from the human or animal body (if indeed they occur naturally), or refers to such molecules when produced by a technical process, i.e. includes recombinant and synthetically produced molecules.

Thus, when used in connection with a protein or polypeptide molecule such as isolated peptides, light chain CDRs 1, 2 and 3, heavy chain CDRs 1, 2 and 3, light chain variable regions, heavy chain variable regions, and binding proteins or antibodies of the invention, including full length antibodies, the term “isolated” or “purified” typically refers to a protein substantially free of cellular material or other proteins from the source from which it is derived. In some embodiments, particularly where the protein is to be administered to humans or animals, such isolated or purified proteins are substantially free of culture medium when produced by recombinant techniques, or chemical precursors or other chemicals when chemically synthesized.

The term “fragment” as used herein refers to fragments of biological relevance, e.g. fragments that contribute to antigen binding, e.g. form part of the antigen binding site, and/or contribute to the functional properties of the KRAS antibody in accordance with the invention. Certain preferred fragments comprise a heavy chain variable region (V_{H} domain) and/or a light chain variable region (V_{L} domain) of the antibodies of the invention.

A person skilled in the art will appreciate that the antibodies, antibody fragments, and immunoconjugates of the invention may be prepared in any of several ways well known and described in the art. For example, polyclonal antibodies may be prepared by immunizing an animal (non-human animal e.g. a rabbit) with an isolated peptide or conjugate of the invention and isolating (and optionally purifying) antibodies to the isolated peptide or conjugate that have been generated by the animal. In other embodiments, antibodies, antibody fragments, and immunoconjugates of the invention may be prepared by recombinant methods.

Nucleic acid fragments encoding the light and heavy chain variable regions of the antibodies of the invention can be derived or produced by any appropriate method, e.g. by cloning or synthesis.

Once nucleic acid fragments encoding the light and heavy chain variable regions of the antibodies of the invention have been obtained, these fragments can be further manipulated by standard recombinant DNA techniques, for example to convert the variable region fragments into full length antibody molecules with appropriate constant region domains, or into particular formats of antibody fragment discussed elsewhere herein, e.g. Fab fragments, scFv fragments, etc. Typically, or as part of this further manipulation procedure, the nucleic acid fragments encoding antibody molecules of the invention are generally incorporated into one or more appropriate expression vectors in order to facilitate production of the antibodies of the invention.

Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner that allows expression of the nucleic acid.

The invention therefore contemplates a recombinant expression vector containing a nucleic acid molecule of the invention, or a fragment thereof, and the necessary regulatory sequences for the transcription and translation of the protein sequence encoded by the nucleic acid molecule of the invention.

Suitable regulatory sequences may be derived from a variety of sources, including bacterial, fungal, viral, mammalian, or insect genes and are well known in the art. Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors of the invention may also contain a selectable marker gene that facilitates the selection of host cells transformed or transfected with a recombinant molecule of the invention.

The recombinant expression vectors may also contain genes that encode a fusion moiety that provides increased expression of the recombinant protein; increased solubility of the recombinant protein; and aid in the purification of the target recombinant protein by acting as a ligand in affinity purification (for example appropriate “tags” to enable purification and/or identification may be present, e.g., His tags or myc tags).

Recombinant expression vectors can be introduced into host cells to produce a transformed host cell. The terms “transformed with”, “transfected with”, “transformation” and “transfection” are intended to encompass introduction of nucleic acid (e.g., a vector) into a cell by one of many possible techniques known in the art. Suitable methods for transforming and transfecting host cells can be found in Sambrook et al., 1989 (Sambrook, Fritsch and Maniatis, Molecular Cloning: A Laboratory Manual, 2nd Ed., Cold Spring Harbor Press, Cold Spring Harbor, N.Y., 1989) and other laboratory textbooks.

Suitable host cells include a wide variety of eukaryotic host cells and prokaryotic cells. For example, the proteins (e.g. antibodies) of the invention may be expressed in yeast cells or mammalian cells. In addition, the proteins of the invention may be expressed in prokaryotic cells, such as Escherichia coli.

Given the teachings provided herein, promoters, terminators, and methods for introducing expression vectors of an appropriate type into plant, avian, and insect cells may also be readily accomplished.

Alternatively, the proteins (e.g. antibodies) of the invention may also be expressed in non-human transgenic animals such as, rats, rabbits, sheep and pigs.

The proteins of the invention may also be prepared by chemical synthesis using techniques well known in the chemistry of proteins such as solid phase synthesis.

N-terminal or C-terminal fusion proteins comprising the antibodies and proteins (e.g. isolated peptides) of the invention conjugated to other molecules, such as proteins, may be prepared by fusing through recombinant techniques. The resultant fusion proteins contain an antibody or protein of the invention fused to the selected protein or marker protein, or tag protein as described herein. The antibodies and proteins of the invention may also be conjugated to other proteins by known techniques. For example, the proteins may be coupled using heterobifunctional thiol-containing linkers as described in WO 90/10457, N-succinimidyl-3-(2-pyridyldithio-proprionate) or N-succinimidyl-5 thioacetate.

A yet further aspect provides an expression construct or expression vector comprising one or more of the nucleic acid fragments or segments or molecules of the invention. Preferably the expression constructs or vectors are recombinant. Also provided is a set of expression vectors (or a set of expression constructs) which, together (collectively), encode an antibody of the invention. Such a set of expression vectors may be characterised in that when the set is expressed (i.e. expressed together) (e.g. in a host cell) an antibody (an entire antibody) of the present invention is expressed and preferably assembled.

Preferably said constructs or vectors further comprise the necessary regulatory sequences for the transcription and translation of the protein sequence encoded by the nucleic acid molecule of the invention.

A yet further aspect provides a host cell or virus comprising one or more expression constructs or expression vectors of the invention. Also provided are host cells or viruses comprising one or more of the nucleic acid molecules of the invention. A host cell (e.g. a mammalian host cell) or virus expressing an antibody of the invention forms a yet further aspect.

A yet further aspect of the invention provides a method of producing (or manufacturing or isolating or identifying or generating) an antibody of the present invention, said method employing an isolated peptide or conjugate of the invention Alternatively viewed, the present invention provides the use of an isolated peptide or conjugate of the invention for the identification (or isolation or generation or production) of an antibody of the invention.

A yet further aspect of the invention provides a method of producing (or manufacturing or isolating or identifying or generating) an antibody of the present invention comprising a step of immunizing an animal (non-human animal e.g. a rabbit) with an isolated peptide (or conjugate) of the invention. Preferred methods include a step of obtaining from said animal antibodies that have been generated (or raised) against the isolated peptide (or conjugate) of the invention, and optionally a step of purification of the antibody product and/or formulating the antibody or product into a composition including at least one additional component, such as a pharmaceutically acceptable carrier or excipient.

A yet further aspect of the invention provides a method of producing (or manufacturing or isolating or identifying or generating) an antibody of the present invention by employing an isolated peptide or conjugate of the invention in hybridoma technology (e.g. conventional hybridoma technology). Alternatively viewed, the present invention provides the use of an isolated peptide or conjugate of the invention for the identification (or isolation or generation or production) of an antibody of the invention using hybridoma technology. In some embodiments, a non-human animal (e.g. mouse) is immunized with an isolated peptide or conjugate of the invention, spleen cells are isolated from said immunized animal (e.g. mouse) and fused with myeloma cells (e.g. mouse myeloma cells) lacking HGPRT expression (such myeloma cells are unable to grow in HAT containing media) and hybrid (i.e. fused or hybridoma) cells are selected using hypoxanthine, aminopterin and thymine (HAT) containing media. Only fused cells grow in HAT containing media.

A yet further aspect of the invention provides a method of identifying (or isolating or generating) an antibody of the invention which employs phage display technology (with a phage display antibody library). Alternatively viewed, the present invention provides the use of an isolated peptide or conjugate of the invention for the identification (or isolation or generation or production) of an antibody of the invention using phage display technology (with a phage display antibody library). In some embodiments, an isolated peptide or conjugate of the invention (typically immobilised on a solid support such as a bead or microbead or plate or microtitre plate) is contacted with phage library (bacteriophage library typically a filamentous bacteriophage library such as an M13 of fd phage library) which displays (or presents or expresses) on the phage surface a library of antibodies or antibody fragments such as scFv or Fab fragments. Any suitable phage display antibody library may be used and the skilled person is familiar with these (and e.g. there are commercially available phage display antibody libraries). The bound phage is then eluted and the identity of the displayed antibody may be readily determined by isolating and sequencing the phage's nucleic acid (or at least the portion of the nucleic acid that encodes the displayed antibody). In some embodiments, after elution of the bound phage, one or more (e.g. 1, 2, 3, 4, 5 or more) additional rounds of contacting and eluting is performed prior to identifying the displayed antibody of the bound phage. Such additional rounds typically further enrich the library. In some typical embodiments, where the peptide or conjugate of the invention is immobilised on a solid support, the solid support is typically washed after the contacting step (and prior to the eluting step), with those phage displaying antibodies or antibody fragments that bind to the immobilised isolated peptide or conjugate remaining bound (and the others being washed away).

A yet further aspect of the invention provides a method of producing (or manufacturing) an antibody of the present invention comprising a step of culturing the host cells of the invention. Preferred methods comprise the steps of (i) culturing a host cell comprising one or more of the recombinant expression vectors (or a set of expression vectors) or one or more of the nucleic acid sequences or molecules (or a set of nucleic acid molecules) of the invention under conditions suitable for the expression of the encoded antibody; and optionally (ii) isolating or obtaining the antibody from the host cell or from the growth medium/supernatant.

In embodiments when the antibody or protein of the invention is made up of more than one polypeptide chain (e.g. certain fragments such as Fab fragments or whole antibodies), then all the polypeptides are preferably expressed in the host cell, either from the same or a different expression vector, so that the complete proteins, e.g. antibody proteins of the invention, can assemble in the host cell and be isolated or purified therefrom.

In some embodiments, methods of producing (or manufacturing or isolating or identifying or generating) an antibody in accordance with the invention may also comprise a step of purification of the antibody or protein product and/or formulating the antibody or product into a composition including at least one additional component, such as a pharmaceutically acceptable carrier or excipient.

In another aspect, the invention provides a method of binding an oncogenic mutant form of KRAS in accordance with the invention, comprising contacting a composition comprising an oncogenic mutant form of KRAS in accordance with the invention with an antibody of the invention, or an immunoconjugate thereof.

In yet another aspect, the invention provides a method of detecting an oncogenic mutant form of KRAS in accordance with the invention, comprising contacting a composition suspected of containing an oncogenic mutant form of KRAS in accordance with the invention with an antibody of the invention, or an immunoconjugate thereof, under conditions effective to allow the formation of KRAS/antibody complexes and detecting the complexes so formed.

Testing the ability of one or more antibodies to bind to an oncogenic mutant form of KRAS can be carried out by any appropriate method, which are well known and described in the art. Suitable methods are also described in the Examples section.

The invention also provides a range of conjugated antibodies and fragments thereof in which the anti-KRAS antibody in accordance with the invention is operatively attached to at least one other therapeutic agent. The term “immunoconjugate” is broadly used to define the operative association of the antibody with another effective agent (e.g. therapeutic agent) and is not intended to refer solely to any type of operative association, and is particularly not limited to chemical “conjugation”. Recombinant fusion proteins are particularly contemplated. So long as the delivery or targeting agent is able to bind to the target and the therapeutic or diagnostic agent is sufficiently functional upon delivery, the mode of attachment will be suitable.

In some embodiments, antibodies of the invention are used (e.g. used therapeutically) in their “naked” unconjugated form.

Compositions comprising at least a first antibody of the invention or an immunoconjugate thereof constitute a further aspect of the present invention. Formulations (compositions) comprising one or more antibodies of the invention in admixture with a suitable diluent, carrier or excipient constitute a preferred embodiment of the present invention. Such formulations may be for pharmaceutical use and thus compositions of the invention are preferably pharmaceutically acceptable. Suitable diluents, excipients and carriers are known to the skilled man.

In some embodiments, compositions of the invention may comprise more than one different antibody of the invention (e.g. 2 or 3 or more). For example, in some embodiments, compositions may comprise more than one of the monoclonal antibodies described herein.

The compositions according to the invention may be presented, for example, in a form suitable for oral, nasal, parenteral, intraperitoneal, intravenal, topical or rectal administration. In some embodiments, a form suitable for intravenal administration is preferred.

The active compounds defined herein may be presented in the conventional pharmacological forms of administration, such as tablets, coated tablets, nasal sprays, solutions, emulsions, liposomes, powders, capsules or sustained release forms. Conventional pharmaceutical excipients as well as the usual methods of production may be employed for the preparation of these forms.

Injection solutions may, for example, be produced in the conventional manner, such as by the addition of preservation agents, such as p-hydroxybenzoates, or stabilizers, such as EDTA. The solutions may then be filled into injection vials or ampoules.

Nasal sprays may be formulated similarly in aqueous solution and packed into spray containers, either with an aerosol propellant or provided with means for manual compression.

The pharmaceutical compositions (formulations) of the present invention are preferably administered parenterally. Intravenous administration is preferred in some embodiments. Parenteral administration may be performed by subcutaneous, intramuscular or intravenous injection by means of a syringe. Alternatively, parenteral administration can be performed by means of an infusion pump. A further option is a composition which may be a powder or a liquid for the administration of the antibody in the form of a nasal or pulmonal spray. As a still further option, the antibodies of the invention can also be administered transdermally, e.g. from a patch, optionally an iontophoretic patch, or transmucosally, e.g. bucally.

Suitable dosage units can be determined by a person skilled in the art.

The pharmaceutical compositions may additionally comprise further active ingredients in the context of co-administration regimens or combined regimens.

A further aspect of the present invention provides an antibody of the invention for use in therapy.

In particular, the present invention provides the antibodies of the invention for use in the therapy of a disease or condition that is associated with (or caused by, or characterised by) the expression of an oncogenic mutant form of KRAS in accordance with the invention

In preferred embodiments, the present invention provides the antibodies of the invention in particular for use in cancer therapy.

Typically, cancer to be treated (or prevented) in accordance with the present invention is cancer that is associated with (or caused by, or characterised by) the expression of an oncogenic mutant form of KRAS in accordance with the invention.

In some embodiments, the cancer is a solid tumour. In some embodiments the cancer is a carcinoma.

Types of cancer to be treated in accordance with the present invention include, but are not limited to, colorectal cancer (e.g. colorectal carcinoma), lung cancer (e.g. non-small cell lung carcinoma) and pancreatic cancer (e.g. pancreatic ductal adenocarcinoma).

“Therapy” includes treatment and prophylaxis, i.e. in includes both treatment and preventative uses. “Cancer therapy” thus includes the treatment of cancer and the prevention of cancer.

Thus, in some embodiments, the present invention provides the antibodies of the invention for use in the treatment of cancer. In some embodiments, the present invention provides the anti-KRAS antibodies of the invention for use in the prevention of cancer.

A further aspect of the present invention provides the antibodies of the invention for use in inhibiting the activity an oncogenic mutant form of KRAS in accordance with the invention (e.g. in a subject). Activity may be as described elsewhere herein.

In another aspect, the present invention provides immunoconjugates of the invention for use in therapy, in particular for use in the treatment of cancer.

The in vivo methods and uses as described herein are generally carried out in a mammal. Any mammal may be treated, for example humans and any livestock, domestic or laboratory animal. Specific examples include mice, rats, pigs, cats, dogs, sheep, rabbits, cows and monkey. Preferably, however, the mammal is a human.

Thus, the term “animal” or “patient” as used herein includes any mammal, for example humans and any livestock, domestic or laboratory animal. Specific examples include mice, rats, pigs, cats, dogs, sheep, rabbits, cows and monkey. Preferably, however, the animal or patient is a human subject. Thus, subjects or patients treated in accordance with the present invention will preferably be humans.

In some embodiments, subjects or patients will be those having cancer, or those at risk of having or developing cancer, or those suspected of having cancer.

Alternatively viewed, the present invention provides a method of treating cancer which method comprises administering to a patient in need thereof a therapeutically effective amount of an antibody of the invention as defined herein. Embodiments of the therapeutic uses of the invention described herein apply, mutatis mutandis, to this aspect of the invention.

The present invention also provides a method of treating a disease or condition (e.g. cancer) that is associated with (or caused by, or characterised by) the expression of an oncogenic mutant form of KRAS in accordance with the invention, which method comprises administering to a patient in need thereof a therapeutically effective amount of an antibody of the invention as defined herein. Embodiments of the therapeutic uses of the invention described herein apply, mutatis mutandis, to this aspect of the invention.

A therapeutically effective amount will be determined based on the clinical assessment and can be readily monitored.

Further alternatively viewed, the present invention provides the use of an antibody of the invention as defined herein in the manufacture of a medicament for use in therapy. Preferred therapy is treatment of a condition that is associated with (or caused by, or characterised by) the expression of an oncogenic mutant form of KRAS in accordance with the invention. A particularly preferred therapy is cancer therapy (typically cancer that is associated with (or caused by, or characterised by) the expression of an oncogenic mutant form of KRAS in accordance with the invention). Embodiments of the therapeutic uses of the invention described herein apply, mutatis mutandis, to this aspect of the invention.

The antibodies and compositions and methods and uses of the present invention may be used in combination with other therapeutics and diagnostics. In terms of biological agents, preferably diagnostic or therapeutic agents, for use “in combination” with an antibody of the present invention, the term “in combination” is succinctly used to cover a range of embodiments. The “in combination” terminology, unless otherwise specifically stated or made clear from the scientific terminology, thus applies to various formats of combined compositions, pharmaceuticals, cocktails, kits, methods, and first and second medical uses.

The “combined” embodiments of the invention thus include, for example, where an antibody of the invention is a naked antibody and is used in combination with an agent or therapeutic agent that is not operatively attached thereto. In other “combined” embodiments of the invention, an antibody of the invention is an immunoconjugate wherein the antibody is itself operatively associated or combined with an agent or therapeutic agent. The operative attachment includes all forms of direct and indirect attachment as described herein and known in the art.

The “combined” uses, particularly in terms of an anti-KRAS antibody of the invention in combination with therapeutic agents, also include combined compositions, pharmaceuticals, cocktails, kits, methods, and first and second medical uses wherein the therapeutic agent is in the form of a prodrug. In such embodiments, the activating component able to convert the prodrug to the functional form of the drug may again be operatively associated with the anti-KRAS antibodies of the present invention.

Thus, where combined compositions, pharmaceuticals, cocktails, kits, methods, and first and second medical uses are described, preferably in terms of diagnostic agents, and more preferably therapeutic agents, the combinations include anti-KRAS antibodies of the invention that are naked antibodies and immunoconjugates, and wherein practice of the in vivo embodiments of the invention involves the prior, simultaneous or subsequent administration of the naked antibodies or immunoconjugate and the biological, diagnostic or therapeutic agent; so long as, in some conjugated or unconjugated form, the overall provision of some form of the antibody and some form of the biological, diagnostic or therapeutic agent is achieved.

The foregoing and other explanations of the effects of the present invention are made for simplicity to explain the combined mode of operation, type of attached agent(s) and such like. This descriptive approach should not be interpreted as either an understatement or an oversimplification of the beneficial properties of the anti-KRAS antibodies of the invention. It will therefore be understood that such antibodies themselves have anti-KRAS properties in accordance with the invention and that immunoconjugates of such antibodies will maintain these properties and combine them with the properties of the attached agent; and further, that the combined effect of the antibody and any attached agent may be enhanced and/or magnified.

The invention therefore provides compositions, pharmaceutical compositions, therapeutic kits and medicinal cocktails comprising, optionally in at least a first composition or container, a biologically effective amount of at least a first antibody of the invention, or an antigen-binding fragment or immunoconjugate of such an antibody of the invention; and a biologically effective amount of at least a second biological agent, component or system.

The “at least a second biological agent, component or system” will often be a therapeutic or diagnostic agent, component or system, but it need not be. For example, the at least a second biological agent, component or system may comprise components for modification of the antibody and/or for attaching other agents to the antibody.

Where therapeutic or diagnostic agents are included as the at least a second biological agent, component or system, such therapeutics and/or diagnostics will typically be those for use in connection with the treatment or diagnosis of one or more of the disorders defined above.

Thus, in certain embodiments “at least a second therapeutic agent” will be included in the therapeutic kit or cocktail. The term is chosen in reference to the anti-KRAS antibody of the invention being the first therapeutic agent.

In certain embodiments of the present invention, the second therapeutic agent may be a further cancer therapy agent or an agent for the treatment of disease that is associated with (or characterised by) cancer.

In terms of compositions, kits and/or medicaments of the invention, the combined effective amounts of the therapeutic agents may be comprised within a single container or container means, or comprised within distinct containers or container means. The cocktails will generally be admixed together for combined use. Agents formulated for intravenous administration will often be preferred. Imaging components may also be included. The kits may also comprise instructions for using the at least a first antibody and the one or more other biological agents included.

Speaking generally, the at least a second therapeutic agent may be administered to the animal or patient substantially simultaneously with the anti-KRAS antibody of the invention; such as from a single pharmaceutical composition or from two pharmaceutical compositions administered closely together.

Alternatively, the at least a second therapeutic agent may be administered to the animal or patient at a time sequential to the administration of the anti-KRAS antibody of the invention. “At a time sequential”, as used herein, means “staggered”, such that the at least a second therapeutic agent is administered to the animal or patient at a time distinct to the administration of the anti-KRAS antibody of the invention. Generally, the two agents are administered at times effectively spaced apart to allow the two agents to exert their respective therapeutic effects, i.e., they are administered at “biologically effective time intervals”. The at least a second therapeutic agent may be administered to the animal or patient at a biologically effective time prior to the anti-KRAS antibody of the invention, or at a biologically effective time subsequent to that therapeutic.

A yet further aspect is a method of imaging of a subject or sample comprising the administration of an appropriate amount of an antibody or other protein of the invention as defined herein to the subject or sample and detecting the presence and/or amount and/or the location of the antibody or other protein of the invention in the subject or sample.

For use in the imaging applications, the antibodies of the invention may be labeled with a detectable marker such as a radio-opaque or radioisotope, such as ³H, ¹⁴C ³²P, ³⁵S, ¹²³I, ¹²⁵I, ¹³¹I; a radioactive emitter (e.g. α, β or γ emitters); a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion; or a chemical moiety such as biotin which may be detected by binding to a specific cognate detectable moiety, e.g. labelled avidin/streptavidin. Methods of attaching a label to a binding protein, such as an antibody or antibody fragment, are known in the art. Such detectable markers allow the presence, amount or location of binding protein-antigen complexes in the test sample to be examined.

The invention also includes imaging agents comprising an antibody of the invention attached to a label that produces a detectable signal, directly or indirectly. Appropriate labels are described elsewhere herein.

The invention further includes kits comprising one or more of the isolated peptides (isolated epitopes), antibodies, immunoconjugates or compositions of the invention or one or more of the nucleic acid molecules encoding the antibodies of the invention, or one or more recombinant expression vectors comprising the nucleic acid sequences of the invention, or one or more host cells or viruses comprising the recombinant expression vectors or nucleic acid sequences of the invention. Preferably said kits are for use in the methods and uses as described herein, e.g. the therapeutic, methods as described herein, or are for use in the in vitro assays or methods as described herein. The antibody in such kits may be a “naked” antibody or may be an antibody conjugate as described elsewhere herein, e.g. may be an immunoconjugate. Preferably said kits comprise instructions for use of the kit components. Preferably said kits are for treating diseases as described elsewhere herein, and optionally comprise instructions for use of the kit components to treat such diseases.

The antibodies of the invention as defined herein may also be used as molecular tools for in vitro or in vivo applications and assays. As the antibodies have an antigen binding site, these can function as members of specific binding pairs and these molecules can be used in any assay where the particular binding pair member is required.

Thus, yet further aspects of the invention provide a reagent that comprises an antibody of the invention as defined herein and the use of such antibodies as molecular tools, for example in in vitro or in vivo assays.

List and Tables of Amino Acid (Aa) and Nucleotide (Nt) Sequences Disclosed Herein and their Sequence Identifiers (SEQ ID NOS)
Amino acid sequence of isolated G12D Ab1 and G12D Ab2 peptides, not including additional modifications present for the purposes of antibody generation (SEQ ID NO:1)

GADGVGKSALTI

Amino acid sequence of isolated G13D Ab1 and G13D Ab2 peptides, not including additional modifications present for the purposes of antibody generation (SEQ ID NO:2)

GAGDVGKSALTI

Amino acid sequence of isolated G12D Ab peptide, including additional modifications present for the purposes of antibody generation (SEQ ID NO:3)

Ac-GADGVGKSALTIC-CONH₂

As compared to SEQ ID NO:1, this peptide has an additional cysteine residue at the C-terminus, is amidated (—CONH₂) at the C-terminus, and is acetylated (Ac-) at the N-terminus.
Amino acid sequence of isolated G12D Ab2 peptide, including additional modifications present for the purposes of antibody generation (SEQ ID NO:4)

### CGADGVGKSALTI-CONH₂

As compared to SEQ ID NO:1, this peptide has an additional cysteine residue at the N-terminus and is amidated (—CONH₂) at the C-terminus.
Amino acid sequence of isolated G13D Ab1 peptide, including additional modifications present for the purposes of antibody generation (SEQ ID NO:5)

CGAGDVGKSALTI-CONH₂

As compared to SEQ ID NO:2, this peptide has an additional cysteine residue at the N-terminus and is amidated (—CONH₂) at the C-terminus.
Amino acid sequence of isolated G13D Ab2 peptide, including additional modifications present for the purposes of antibody generation (SEQ ID NO:6)

Ac-GAGDVGKSALTIC-CONH₂

As compared to SEQ ID NO:2, this peptide has an additional cysteine residue at the C-terminus, is amidated (—CONH₂) at the C-terminus, and is acetylated (Ac-) at the N-terminus.

Amino acid sequence of human wildtype KRAS (Isoform 2A) (SEQ ID NO:7)

MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM

Amino acid sequence of human G12D mutant KRAS (Isoform 2A) (SEQ ID NO:8)

MTEYKLVVVGADGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM

Amino acid sequence of human G13D mutant KRAS (Isoform 2A) (SEQ ID NO:9)

MTEYKLVVVGAGDVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM

Amino acid sequence of human wildtype KRAS (Isoform 2B) (SEQ ID NO:10)

MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM

Amino acid sequence of human G12D mutant KRAS (Isoform 2B) (SEQ ID NO:11)

MTEYKLVVVGADGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM

Amino acid sequence of human G13D mutant KRAS (Isoform 2B) (SEQ ID NO:12)

MTEYKLVVVGAGDVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM

Amino acid sequence of a human G12X mutant KRAS (Isoform 2A) (SEQ ID NO:13)

MTEYKLVVVGAXGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM

wherein residue “X” is D, V, C, A, S or R

Amino acid sequence of a human G12X mutant KRAS (Isoform 2B) (SEQ ID NO:14)

MTEYKLVVVGAGXVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM

wherein residue “X” is D or C

Amino acid sequence of a human G12X mutant KRAS (Isoform 2B) (SEQ ID NO:15)

MTEYKLVVVGAXGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM

wherein residue “X” is D, V, C, A, S or R

Amino acid sequence of human G13X mutant KRAS (Isoform 2B) (SEQ ID NO:16)

MTEYKLVVVGAGXVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM

wherein residue “X” is D or C
Amino acid sequence of an isolated G12X peptide. (SEQ ID NO:17)

GAXGVGKSALTI

wherein residue “X” is D, V, C, A, S or R
Amino acid sequence of isolated G13X peptide, (SEQ ID NO:18)

GAGXVGKSALTI

wherein residue “X” is D or C
Amino acid sequence of an isolated G12X peptide, (SEQ ID NO:19)

GAXGVGKSALTI

wherein residue “X” is D, V, C, A or R
Amino acid sequence of an isolated G12X peptide, (SEQ ID NO:20)

GAXGVGKSALTI

wherein residue “X” is D, C, A, S or R
Amino acid sequence of an isolated G12X peptide, (SEQ ID NO:21)

GAXGVGKSALTI

wherein residue “X” is D, C, A or R
Amino acid sequence of a human G12X mutant KRAS (Isoform 2A) (SEQ ID NO:22)

MTEYKLVVVGAXGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM

wherein residue “X” is D, V, C, A or R
Amino acid sequence of a human G12X mutant KRAS (Isoform 2B) (SEQ ID NO:23)

MTEYKLVVVGAXGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM

wherein residue “X” is D, V, C, A or R

Amino acid sequence of a human G12X mutant KRAS (Isoform 2A) (SEQ ID NO:24)

MTEYKLVVVGAXGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM

wherein residue “X” is D, C, A, S or R

Amino acid sequence of a human G12X mutant KRAS (Isoform 2B) (SEQ ID NO:25)

MTEYKLVVVGAXGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM

wherein residue “X” is D, C, A, S or R

Amino acid sequence of a human G12X mutant KRAS (Isoform 2A) (SEQ ID NO:26)

MTEYKLVVVGAXGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM

wherein residue “X” is D, C, A or R

Amino acid sequence of a human G12X mutant KRAS (Isoform 2B) (SEQ ID NO:27)

MTEYKLVVVGAXGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM

wherein residue “X” is D, C, A or R

TABLE A
Sequences of antibody 11D6-1
Table A

| SEQ ID | | | |
|---|---|---|---|
| NO: | Description | | Sequence |
| 11D6-1 | | | |
| 28 | VH domain (nt) | | GAGGTCCAGCTGCAACAGTCTGGACCTGAACTGGTGAAG |
| | | | CCTGGGGCTTCAGTGAAGATGTCCTGCAAGACTTCTGGA |
| | | | TACACATTCACTGACTACACCATGCACTGGGTGAAGCAG |
| | | | AGCCATGGAAAGAGCCTTGAGTGGATTGGATATATTAAC |
| | | | CCTAACAATGGTGGAACTTACTACAACCAGAGGTTCAAG |
| | | | GGCAAGGCCACATTGACTGTAAGCAGGTCCTCCAGCACA |
| | | | GCCTACATAGAGCTCCGCAGCCTGACATCGGACGATTCT |
| | | | GCAGTCTATTACTGTGCACGAGGGGATTACTACGGTGGT |
| | | | GGCTACTGGGGCCAAGGCACCACGCTCAGAGTCTCCTCA |
| 29 | VL domain (nt) | | GAGAATGTTCTCACCCAGTCTCCAGCAATCATGTCTGCAT |
| | | | CTCCAGGGGAAAAGGTCACCATGACCTGCAGTGCCGGCT |
| | | | CAAGTGTAAGTTACATGCACTGGTATCAGCAGAAGTCAA |
| | | | GCACCTCCCCCAAACTCTGGATTTATGACACATCCAAACT |
| | | | GGCTTCTGGAGTCCCGGGTCGCTTCAGTGGCAGTGGGTC |
| | | | TGGAAACTCTTACTCTCTCACGATCAGCAGCATGGAGGCT |
| | | | GAAGATGTTGCCACTTATTACTGTTTTCAGGGGAGTGGG |
| | | | TACCCACTCACGTTCGGTGATGGGACCAAGCTGGAGCTG |
| | | | AAA |
| 30 | VH domain (aa) | | EVQLQQSGPELVKPGASVKMSCKTSGYTFTDYTMHWVKQ |
| | | | SHGKSLEWIGYINPNNGGTYYNQRFKGKATLTVSRSSSTAYI |
| | | | ELRSLTSDDSAVYYCARGDYYGGGYWGQGTTLRVSS |
| 31 | VL domain (aa) | | ENVLTQSPAIMSASPGEKVTMTCSAGSSVSYMHWYQQKSS |
| | | | TSPKLWIYDTSKLASGVPGRFSGSGSGNSYSLTISSMEAEDV |
| | | | ATYYCFQGSGYPLTFGDGTKLELK |
| 32 | Heavy CDR1 | | DYTMH |
| 33 | Heavy CDR2 | | YINPNNGGTYYNQRFKG |
| 34 | | Heavy CDR3 | GDYYGGGY |

| | | |
|---|---|---|
| 35 | Light CDR1 | SAGSSVSYMH |
| 36 | Light CDR2 | DTSKLAS |
| 37 | Light CDR3 | FQGSGYPLT |
| 38 | Heavy FR1 | EVQLQQSGPELVKPGASVKMSCKTSGYTFT |
| 39 | Heavy FR2 | WVKQSHGKSLEWIG |
| 40 | Heavy FR3 | KATLTVSRSSSTAYIELRSLTSDDSAVYYCAR |
| 41 | Heavy FR4 | WGQGTTLRVSS |
| 42 | Light FR1 | ENVLTQSPAIMSASPGEKVTMTC |
| 43 | Light FR2 | WYQQKSSTSPKLWIY |
| 44 | Light FR3 | GVPGRFSGSGSGNSYSLTISSMEAEDVATYYC |
| 45 | Light FR4 | FGDGTKLELK |
| 46 | VH Signal peptide | MGWSWIFLFLLSETAGVLS |
| 47 | VL Signal peptide | MDFQVQIFSFLLISASVIMSRG |

TABLE B
Sequences of antibody 4B8-1
Table B

| SEQ ID | | |
|---|---|---|
| NO: | Description | Sequence |
| 4B8-1 | | |
| 48 | VH domain (nt) | GAGGTCCAGCTGCAACAGTCAGGACCTGAGCGGGTGAA |
| | | GCCTGGGGCTTCAGTGAAGATGTCCTGCAAGGCTTCTGG |
| | | ATACACATTCACTGACTACAGCATGCACTGGGTGAAGCA |
| | | GAGCCATGGAAAGAGCCTTGAATGGATTGGATATATTAA |
| | | CCCGAACAATGGTGGTGCTAGCTACAACCAGAAGTTCAA |
| | | GGGCAAGGCCACATTGACTGTAAACAAGTCCTCCAGCAC |
| | | AGCCTACATGGAGCTCCGCAGCCTGACATCGGAGGATTC |
| | | TGCAGTCTATTACTGTGCAAGAGGGGATTACTACGGTGG |
| | | TGGCTACTGGGGCCAAGGCACCACTCTCACAGTCTCCTCA |
| 49 | VL domain (nt) | GAAAATGTTCTCACCCAGTCTCCAGCAATCATGTCTGCAT |
| | | CTCCAGGGGAAAAGGTCACCATGACCTGCAGTGCCAGCT |
| | | CAAGTGTAAGTTACATGCACTGGTACCAGCAGAAGTCAA |
| | | GCACCTCCCCCAAACTCTGGATTTATGACACATCCAAAGT |
| | | GGCTTCTGGAGTCCCAGGTCGCTTCAGTGGCAGTGGGTC |
| | | TGGAAACTCTTACTCTCTCACGATCAACAGCATGGAGGCT |
| | | GAAGATGTTGCCACTTATTACTGTTTTCAGGGGAGTGGG |
| | | TACCCACTCACGTTCGGTGCTGGGACCAAGCTGGAGCTG |
| | | AAA |
| 50 | VH domain (aa) | EVQLQQSGPERVKPGASVKMSCKASGYTFTDYSMHWVKQ |
| | | SHGKSLEWIGYINPNNGGASYNQKFKGKATLTVNKSSSTAY |
| | | MELRSLTSEDSAVYYCARGDYYGGGYWGQGTTLTVSS |
| 51 | VL domain (aa) | ENVLTQSPAIMSASPGEKVTMTCSASSSVSYMHWYQQKSS |
| | | TSPKLWIYDTSKVASGVPGRFSGSGSGNSYSLTINSMEAEDV |
| | | ATYYCFQGSGYPLTFGAGTKLELK |
| 52 | Heavy CDR1 | DYSMH |
| 53 | Heavy CDR2 | YINPNNGGASYNQKFKG |

| | | |
|---|---|---|
| 54 or 34 | Heavy CDR3 | GDYYGGGY |

| | | |
|---|---|---|
| 55 | Light CDR1 | SASSSVSYMH |
| 56 | Light CDR2 | DTSKVAS |
| 57 or 37 | Light CDR3 | FQGSGYPLT |
| 58 | Heavy FR1 | EVQLQQSGPERVKPGASVKMSCKASGYTFT |
| 59 | Heavy FR2 | WVKQSHGKSLEWIG |
| 60 | Heavy FR3 | KATLTVNKSSSTAYMELRSLTSEDSAVYYCAR |
| 61 | Heavy FR4 | WGQGTTLTVSS |
| 62 | Light FR1 | ENVLTQSPAIMSASPGEKVTMTC |
| 63 | Light FR2 | WYQQKSSTSPKLWIY |
| 64 | Light FR3 | GVPGRFSGSGSGNSYSLTINSMEAEDVATYYC |
| 65 | Light FR4 | FGAGTKLELK |
| 66 | VH Signal peptide | MGWSWIFLFLLSETAGVLS |
| 67 | VL Signal peptide | MDFQVQIFSFLLISASVIMSRG |

TABLE C
Sequences of antibody 7D11-1
Table C

| SEQ ID | | |
|---|---|---|
| NO: | Description | Sequence |
| 7D11-1 | | |
| 68 | VH domain (nt) | CAGGTCCAACTGCAGCAGCCTGGGGCTGAGTTTGTGAAG |
| | | CCTGGGGCTTCAGTGAAGCTGTCCTGCAAGGCTTCTGGC |
| | | TACACCTTCACCCGCTACTGGATGCACTGGGTGAAGCAG |
| | | AGGCCTGGACGAGGCCTTGAGTGGATTGGAAGGATTGA |
| | | TCCTAATAGTGGTGGTACTACGTTCAATGAGAAGTTCAA |
| | | GAGCAAGGCCACACTGACTGTAGACAAACCCTCCAGCAC |
| | | AGCCTACATGCAGCTCAGCAGCCTGACATCTGAGGACTC |
| | | TGCGGTCTATTATTGTGCAAGAGAGGTCGGGGGCTACTG |
| | | GGGCCAAGGCACCACTCTCACAGTCTCCTCA |
| 69 | VL domain (nt) | GAAAATGTTCTCACCCAGTCTCCAGCAATCATGTCTGCAT |
| | | CTCCAGGGGAAAAGGTCACCATGACCTGCAGTGCCGGGT |
| | | CAAGTGTAGATTACATGCACTGGTACCAGCAGAAGTCAA |
| | | GCACCTCCCCCAAACTCTGGATTTATGACACATCCAAGCT |
| | | GGCTTCTGGAGTCCCAGGTCGCTTCAGTGGCAGTGGGTC |
| | | TGGAAACTCTTATTCTCTCACGATCAGCAGCATGGAGGCT |
| | | GAAGATGTTGCCACTTATTACTGTTTTCAGGGGAGTGGG |
| | | TACCCACTCACGTTCGGTGCTGGGACCAAGCTGGAGCTG |
| | | AGA |
| 70 | VH domain (aa) | QVQLQQPGAEFVKPGASVKLSCKASGYTFTRYWMHWVKQ |
| | | RPGRGLEWIGRIDPNSGGTTFNEKFKSKATLTVDKPSSTAY |
| | | MQLSSLTSEDSAVYYCAREVGGYWGQGTTLTVSS |
| 71 | VL domain (aa) | ENVLTQSPAIMSASPGEKVTMTCSAGSSVDYMHWYQQKS |
| | | STSPKLWIYDTSKLASGVPGRFSGSGSGNSYSLTISSMEAED |
| | | VATYYCFQGSGYPLTFGAGTKLELR |
| 72 | Heavy CDR1 | RYWMH |
| 73 | Heavy CDR2 | RIDPNSGGTTFNEKFKS |

| | | |
|---|---|---|
| 74 | Heavy CDR3 | EVGGY |

| | | |
|---|---|---|
| 75 | Light CDR1 | SAGSSVDYMH |
| 76 or 36 | Light CDR2 | DTSKLAS |
| 77 or 37 | Light CDR3 | FQGSGYPLT |
| or 57 | | |
| 78 | Heavy FR1 | QVQLQQPGAEFVKPGASVKLSCKASGYTFT |
| 79 | Heavy FR2 | WVKQRPGRGLEWIG |
| 80 | Heavy FR3 | KATLTVDKPSSTAYMQLSSLTSEDSAVYYCAR |
| 81 | Heavy FR4 | WGQGTTLTVSS |
| 82 | Light FR1 | ENVLTQSPAIMSASPGEKVTMTC |
| 83 | Light FR2 | WYQQKSSTSPKLWIY |
| 84 | Light FR3 | GVPGRFSGSGSGNSYSLTISSMEAEDVATYYC |
| 85 | Light FR4 | FGAGTKLELR |
| 86 | VH Signal peptide | MGWSCIMLFLAATATGVHS |
| 87 | VL Signal peptide | MDFQVQIFSFLLISASVIMSRG |

TABLE D
Sequences of antibody 7G2-1
Table D

| SEQ ID | | | |
|---|---|---|---|
| NO: | | Description | Sequence |
| 7G2-1 | | | |
| 88 | VH domain (nt) | | CAGGTTACTCTGAAAGAGTCTGGCCCTGGGATATTGCAG |
| | | | CCCTCCCAGACCCTCAGTCTGACTTGTTCTTTCTCTGGGTT |
| | | | TTCACTGAGCACTTTTGGTATGGGTGTAGGCTGGATTCGT |
| | | | CAGCCTTCAGGGAAGGGTCTGGAGTGGCTGGCACACATT |
| | | | TGGTGGGATGATGATAAGAACTATAACCCAGCCCTGAAG |
| | | | AGTCGGCTCACAATCTCCAAGGATACCTCCAAAAACCAG |
| | | | GTTTTCTTCAAGATCGCCAATGTGGACACTGCAGATACTG |
| | | | CCACATACTACTGTGTTCAACTTTCTACTATGGGAGACGG |
| | | | GGGGTACTGGGGCCAAGGGACTCTGGTCACTGTCTCTGC |
| | | | A |
| 89 | VL domain (nt) | | AACATTGTAATGACCCAATCTCCCAAATCCATGTCCATGT |
| | | | CAGTAGGAGAGAGGGTCACCTTGAGCTGCAAGGCCAGT |
| | | | GAGAATGTGGGTACTTATGTATCCTGGTATCAACAGAAA |
| | | | CCAGGGCAGTCTCCTAAATTGTTGATATACGGGGCATCC |
| | | | AACCGGTACACTGGGGTCCCCGATCGCTTCACAGGCAGT |
| | | | GGATCTGGAACAGATTTCATTCTGACCATCAGCAGTGTGC |
| | | | AGGCTGAAGACCTTGCAGATTATCACTGTGGACAGAGTT |
| | | | ACAACTATCCATTCACGTTCGGCTCGGGGACAAAGTTGG |
| | | | AAATAAAA |
| 90 | VH domain (aa) | | QVTLKESGPGILQPSQTLSLTCSFSGFSLSTFGMGVGWIRQP |
| | | | SGKGLEWLAHIWWDDDKNYNPALKSRLTISKDTSKNQVFF |
| | | | KIANVDTADTATYYCVQLSTMGDGGYWGQGTLVTVSA |
| 91 | VL domain (aa) | | NIVMTQSPKSMSMSVGERVTLSCKASENVGTYVSWYQQK |
| | | | PGQSPKLLIYGASNRYTGVPDRFTGSGSGTDFILTISSVQAED |
| | | | LADYHCGQSYNYPFTFGSGTKLEIK |
| 92 | Heavy CDR1 | | TFGMGVG |
| 93 | | Heavy CDR2 | HIWWDDDKNYNPALKS |

| | | |
|---|---|---|
| 94 | Heavy CDR3 | LSTMGDGGY |
| 95 | Light CDR1 | KASENVGTYVS |
| 96 | Light CDR2 | GASNRYT |
| 97 | Light CDR3 | GQSYNYPFT |
| 98 | Heavy FR1 | QVTLKESGPGILQPSQTLSLTCSFSGFSLS |
| 99 | Heavy FR2 | WIRQPSGKGLEWLA |
| 100 | Heavy FR3 | RLTISKDTSKNQVFFKIANVDTADTATYYCVQ |
| 101 | Heavy FR4 | WGQGTLVTVSA |
| 102 | Light FR1 | NIVMTQSPKSMSMSVGERVTLSC |
| 103 | Light FR2 | WYQQKPGQSPKLLIY |
| 104 | Light FR3 | GVPDRFTGSGSGTDFILTISSVQAEDLADYHC |
| 105 | Light FR4 | FGSGTKLEIK |
| 106 | VH Signal peptide | MGRLTSSFLLLIVPAYVLS |
| 107 | VL Signal peptide | MESQTLVFISILLWLYGADG |

TABLE E
Sequences of antibody 6B2-1-1
Table E

| SEQ ID | | |
|---|---|---|
| NO: | Description | Sequence |
| 6B2-1-1 | | |
| 108 | VH domain (nt) | CAGGTTACTCTGAAAGAGTCTGGCCCTGGGATATTGCAG |
| | | CCCTCCCAGACCCTCAGTCTGACTTGTTCTTTCTCTGGGTT |
| | | TTCACTGAGCACTTTTGGTGTGGGTGTAGCCTGGATTCGT |
| | | CAGCCTTCAGGGAAGGGTCTGGAGTGGCTGGCACACATT |
| | | TGGTGGGATGATGATAAGAACTATGACCCAGCCCTGAAG |
| | | AGTCGGCTCACAATCTCCAAGGATACCTCCAAAAACCAG |
| | | GTTTTCCTCAAGATCGCCAATGTGGACACTACAGATTCTG |
| | | CCACATACTACTGTGCTCGAATAGGATCTACTTTGATTAC |
| | | GCCAGGGGATGCTTCCTGGGGCCAGGGGACTCTGGTCAC |
| | | TGTCTCTGCA |
| 109 | VL domain (nt) | AACATTGTTATGACCCAATCTCCCAAATCCATGTCCATGTC |
| | | AGTAGGAGAGAGGGTCACCTTGAGCTGCAAGGCCAGTG |
| | | AGAATGTGGGTACTTTTGTATCCTGGTATCAACAGAAACC |
| | | AGACCAGTCTCCTAAACTGCTGATATACGGGGCATCCAA |
| | | CCGGTACACTGGGGTCCCCGATCGCTTCACAGGCAGTGG |
| | | ATCTGCAACAGATTTCACTCTGACCATCAGCAGTGTGCAG |
| | | GCTGAAGACCTTGCAGATTATCACTGTGGACAGAGTTAC |
| | | AGCTATCCATACACGTTCGGAGGGGGGACCAAGCTGGA |
| | | AATCAAA |
| 110 | VH domain (aa) | QVTLKESGPGILQPSQTLSLTCSFSGFSLSTFGVGVAWIRQPS |
| | | GKGLEWLAHIWWDDDKNYDPALKSRLTISKDTSKNQVFLKI |
| | | ANVDTTDSATYYCARIGSTLITPGDASWGQGTLVTVSA |
| 111 | VL domain (aa) | NIVMTQSPKSMSMSVGERVTLSCKASENVGTFVSWYQQK |
| | | PDQSPKLLIYGASNRYTGVPDRFTGSGSATDFTLTISSVQAE |
| | | DLADYHCGQSYSYPYTFGGGTKLEIK |
| 112 | Heavy CDR1 | TFGVGVA |

| | | |
|---|---|---|
| 113 | Heavy CDR2 | HIWWDDDKNYDPALKS |

| | | |
|---|---|---|
| 114 | Heavy CDR3 | IGSTLITPGDAS |
| 115 | Light CDR1 | KASENVGTFVS |
| 116 or | Light CDR2 | GASNRYT |
| 96 | | |
| 117 | Light CDR3 | GQSYSYPYT |
| 118 | Heavy FR1 | QVTLKESGPGILQPSQTLSLTCSFSGFSLS |
| 119 | Heavy FR2 | WIRQPSGKGLEWLA |
| 120 | Heavy FR3 | RLTISKDTSKNQVFLKIANVDTTDSATYYCAR |
| 121 | Heavy FR4 | WGQGTLVTVSA |
| 122 | Light FR1 | NIVMTQSPKSMSMSVGERVTLSC |
| 123 | Light FR2 | WYQQKPDQSPKLLIY |
| 124 | Light FR3 | GVPDRFTGSGSATDFTLTISSVQAEDLADYHC |
| 125 | Light FR4 | FGGGTKLEIK |
| 126 | VH Signal peptide | MGRLTSSFLLLIVPAYVLS |
| 127 | VL Signal peptide | MESQTLVFISILLWLFGADG |

TABLE F
Sequences of antibody 6B2-1-2
Table F

| SEQ ID | | |
|---|---|---|
| NO: | Description | Sequence |
| 6B2-1-2 | | |
| 128 | VH domain (nt) | CAGGTTACTCTGAAAGAGTCTGGCCCTGGGATATTGCAG |
| | | CCCTCCCAGACCCTCAGTCTGACTTGTTCTTTCTCTGGGTT |
| | | TTCACTGAGCACTTTTGGTGTGGGTGTAGCCTGGATTCGT |
| | | CAGCCTTCAGGGAAGGGTCTGGAGTGGCTGGCACACATT |
| | | TGGTGGGATGATGATAAGAACTATGACCCAGCCCTGAAG |
| | | AGTCGGCTCACAATCTCCAAGGATACCTCCAAAAACCAG |
| | | GTTTTCCTCAAGATCGCCAATGTGGACACTACAGATTCTG |
| | | CCACATACTACTGTGCTCGAATAGGATCTACTTTGATTAC |
| | | GCCAGGGGATGCTTCCTGGGGCCAGGGGACTCTGGTCAC |
| | | TGTCTCTGCA |
| 129 | VL domain (nt) | GATATTGTACTAACTCAGTCTCCAGCCACCCTGTCTGTGA |
| | | CTCCAGGAGATAGAGTCAGTCTTTCCTGCAGGGCCAGTC |
| | | AAAGTATTAGCGACTACCTACACTGGTATCAACAAAAATC |
| | | ACATGAGTCTCCAAGGCTTCTCATCAAGTTTGCTTCCCAG |
| | | TCCATCTCTGGGATCCCCTCCAGGTTCAGTGGCAGTGGAT |
| | | CAGGGACAGATTTCACTCTCAGTATCAACAGTGTGGAGA |
| | | CTGAAGATTTTGGAATGTATTTCTGTCAACAGAGTAACAA |
| | | CTGGCCTCACATGTACACGTTCGGAGGGGGGACCAAGCT |
| | | GGAAATAAAA |
| 130 | VH domain (aa) | QVTLKESGPGILQPSQTLSLTCSFSGFSLSTFGVGVAWIRQPS |
| | | GKGLEWLAHIWWDDDKNYDPALKSRLTISKDTSKNQVFLKI |
| | | ANVDTTDSATYYCARIGSTLITPGDASWGQGTLVTVSA |
| 131 | VL domain (aa) | DIVLTQSPATLSVTPGDRVSLSCRASQSISDYLHWYQQKSHE |
| | | SPRLLIKFASQSISGIPSRFSGSGSGTDFTLSINSVETEDFGMY |
| | | FCQQSNNWPHMYTFGGGTKLEIK |
| 132 or | Heavy CDR1 | TFGVGVA |
| 112 | | |

| | | |
|---|---|---|
| 133 or | Heavy CDR2 | HIWWDDDKNYDPALKS |
| 113 | | |

| | | |
|---|---|---|
| 134 or | Heavy CDR3 | IGSTLITPGDAS |
| 114 | | |
| 135 | Light CDR1 | RASQSISDYLH |
| 136 | Light CDR2 | FASQSIS |
| 137 | Light CDR3 | QQSNNWPHMYT |
| 138 | Heavy FR1 | QVTLKESGPGILQPSQTLSLTCSFSGFSLS |
| 139 | Heavy FR2 | WIRQPSGKGLEWLA |
| 140 | Heavy FR3 | RLTISKDTSKNQVFLKIANVDTTDSATYYCAR |
| 141 | Heavy FR4 | WGQGTLVTVSA |
| 142 | Light FR1 | DIVLTQSPATLSVTPGDRVSLSC |
| 143 | Light FR2 | WYQQKSHESPRLLIK |
| 144 | Light FR3 | GIPSRFSGSGSGTDFTLSINSVETEDFGMYFC |
| 145 | Light FR4 | FGGGTKLEIK |
| 146 | VH Signal peptide | MGRLTSSFLLLIVPAYVLS |
| 147 | VL Signal peptide | MVFTPQILGLMLFWISASRG |

TABLE G
Consensus amino acid sequences

| SEQ | | |
|---|---|---|
| ID | Descrip- | Sequence |
| NO: | tion | |
| 148 | Heavy | X₁ Y X₃ M H |
| | CDR1 | wherein X₁ or X₃ are any amino acid. |
| 149 | Heavy | X₁ Y X₃ M H |
| | CDR1 | wherein X₁ is D or R, and X₃ is S or T |
| | | or W. |
| 150 | Heavy | X₁ I X₃ P N X₆ G G X₉ X₁₀ X₁₁ N X₁₃ X₁₄ |
| | CDR2 | F K X₁₇ |
| | | wherein X₁, X₃, X₆, X₉, X₁₀, X₁₁, X₁₃, |
| | | X₁₄ and X₁₇ are any amino acid |
| 151 | Heavy | X₁ I X₃ P N X₆ G G X₉ X₁₀ X₁₁ N X₁₃ X₁₄ |
| | CDR2 | F K X₁₇ |
| | | wherein X₁ is Y or R (preferably Y), |
| | | X₃ is N or D (preferably N), X₆ is N |
| | | or S (preferably N), X₉ is A or T, X₁₀ |
| | | is S or Y or T, X₁₁ is Y or F |
| | | (preferably Y), X₁₃ is Q or E |
| | | (preferably Q), X₁₄ is K or R and X₁₇ |
| | | is G or S (preferably G). |
| 152 | Light | S A X₃ S S V X₇ Y M H |
| | CDR1 | wherein X₃ and X₇ are any amino acid. |
| 153 | Light | S A X₃ S S V X₇ Y M H |
| | CDR1 | wherein X₃ is S or G and X₇ is S or D. |
| 154 | Light | D T S K X₅ A S |
| | CDR2 | wherein X₅ is any amino acid. |
| 155 | Light | D T S K X₅ A S |
| | CDR2 | wherein X₅ is V or L. |

The VH (i.e. heavy) CDR1 and CDR2 amino acid sequences and the VL (light) CDR1 and CDR2 sequences of the 4B8-1, 11D6-1 and 7D11-1 antibodies of the invention each fall within the consensus sequences of the above Table G.

TABLE L
Consensus amino acid sequences

| SEQ | | |
|---|---|---|
| ID | Descrip- | |
| NO: | tion | Sequence |
| 156 | Heavy | T F G X₄ G V X₇ |
| | CDR1 | wherein X₄ or X₇ are any amino acid. |
| 157 | Heavy | T F G X₄ G V X₇ |
| | CDR1 | wherein X₄ is V or M, and or X₇ is A |
| | | or G. |
| 158 | Heavy | H I W W D D D K N Y X₁₁ P A L K S |
| | CDR2 | wherein X₁₁ is any amino acid |
| 159 | Heavy | H I W W D D D K N Y X₁₁ P A L K S |
| | CDR2 | wherein X₁₁ is D or N. |

The VH (i.e. heavy) CDR1 and CDR2 amino acid sequences of the 7G2-1, 6B2-1-1 and 6B2-1-2 antibodies of the invention each fall within the consensus sequences of the above Table L.

The invention will now be further described in the following non-limiting Examples with reference to the following drawings:

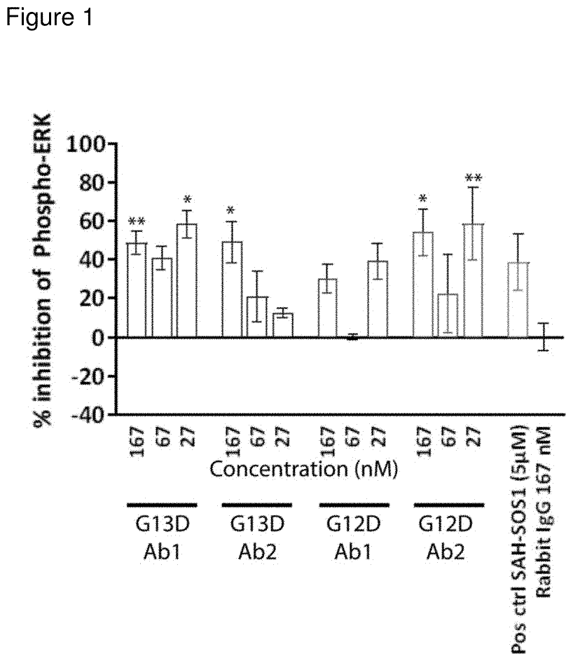

FIG. 1: Inhibition of phosphorylation of ERK in MDA-MB-231 (G13D) cells, induced by four anti-KRAS antibodies, G13D Ab1, G13D Ab2, G12D Ab1 and G12D Ab2. Antibodies were added to the cell medium (14-16 hours incubation) and intracellular phosphorylation of ERK was quantified using Perkin Elmer p-ERK 1/2 AlphaScreen Surefire assay kits. SAH-SOS1 (stabilized alpha helices of son of sevenless 1) was used as a positive control and rabbit IgG as a negative control (isotype control). Statistical significance is indicated as follows: *p<0.05, **p<0.01. Data is presented as mean±SEM. n=3 per treatment group except for G12D Ab2 27 nM and 67 nM where n=2.

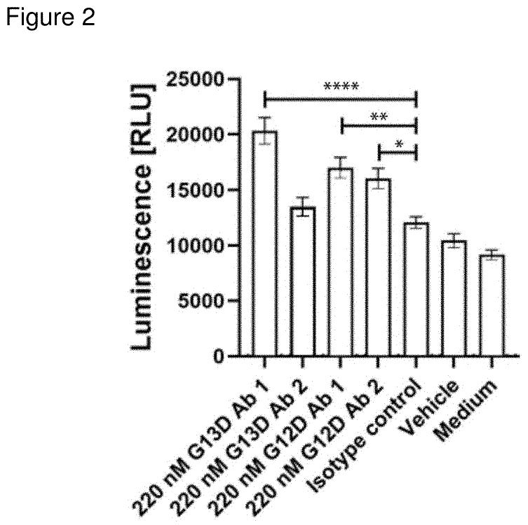

FIG. 2: Determination of apoptosis in G12D mutated SK-LU-1 (lung adenocarcinoma) cells by four anti-KRAS antibodies, G13D Ab1, G13D Ab2, G12D Ab1 and G12D Ab2. Cells were treated with antibody or control and apoptosis was quantified in real time using RealTime-Glo™ Annexin V Apoptosis and Necrosis Assay kit (Promega). The isotype control was rabbit IgG. This graph represents the state of apoptosis at 52 hrs after addition of antibodies/controls to the cells. Luminescence is proportional to the level of apoptosis. Data was collected during a single day, n=4 per treatment. Statistical significance is indicated as follows: *p<0.05, **p<0.01, ****p<0.0001. Data is presented as mean±SEM.

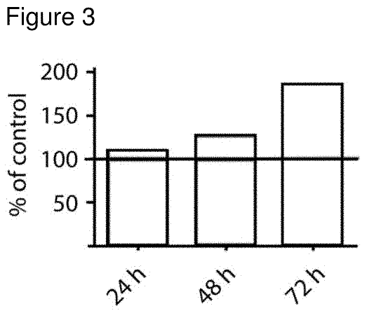

FIG. 3: Determination of apoptosis in G12D mutated LS174T (colon adenocarcinoma) cells, induced by G12D Ab1, at 24, 48 and 72 hrs. Cells were treated with antibody or control and apoptosis was quantified by FACS, using fluorescence labelled Annexin V as the marker of apoptosis. Data is shown as % of control, where the control consisted of cells treated with vehicle (medium). n=1.

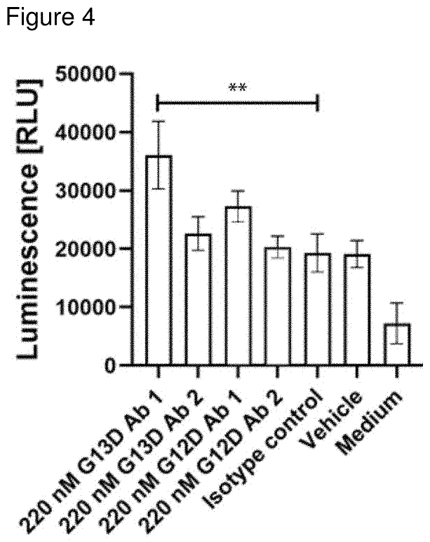

FIG. 4: Determination of apoptosis in G13D mutated HCT116 (colon carcinoma) cells, induced by four anti-KRAS antibodies, G13D Ab1, G13D Ab2, G12D Ab1 and G12D Ab2. Cells were treated with antibody or control and apoptosis was quantified in real time using RealTime-Glo™ Annexin V Apoptosis and Necrosis Assay kit (Promega). The isotype control was rabbit IgG. This graph represents the state of apoptosis at 52 hrs after addition of antibodies/controls to the cells. Luminescence is proportional to the level of apoptosis. Statistical significance is indicated as follows: **p<0.01. Data is presented as mean±SEM. n=3.

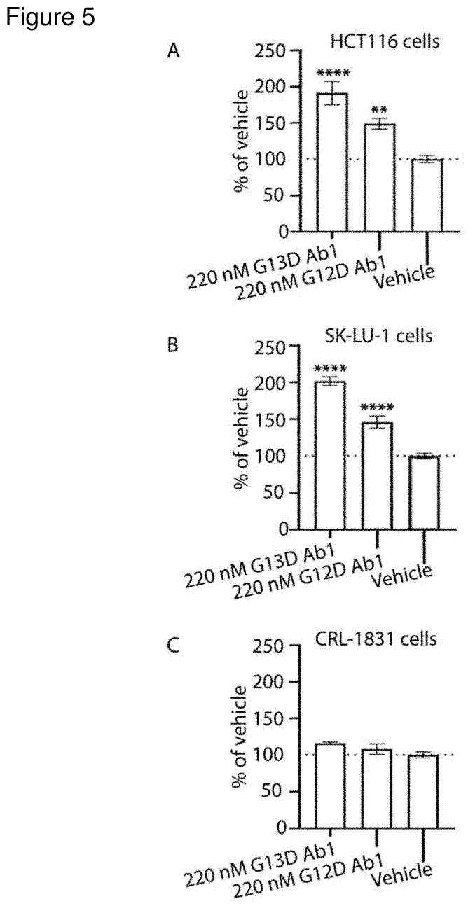

FIG. 5: Determination of apoptosis induced by antibodies G13D Ab1 and G12D Ab2 in (A) G13D mutated HCT116 (colon carcinoma) cells; (B) G12D mutated SK-LU-1 (lung adenocarcinoma) cells; and (C) non-cancerous wtKRAS CRL-1831 (colon epithelia) cells. Cells were treated with antibody or control and apoptosis was quantified in real time using RealTime-Glo™ Annexin V Apoptosis and Necrosis Assay kit (Promega). Vehicle is PBS/Tween. This graph represents the state of apoptosis at 52 hrs after addition of antibodies/control to the cells. For the HCT116 data, data was collected during 3 days with n=4 per day. For the SK-LU-1 data, data was collected during 2 days with n=4 per day. For the CRL-1831, data was collected during 1 day with n=4. Statistical significance is indicated as follows: **p<0.01, ****p<0.0001. Data is presented as mean±SEM.

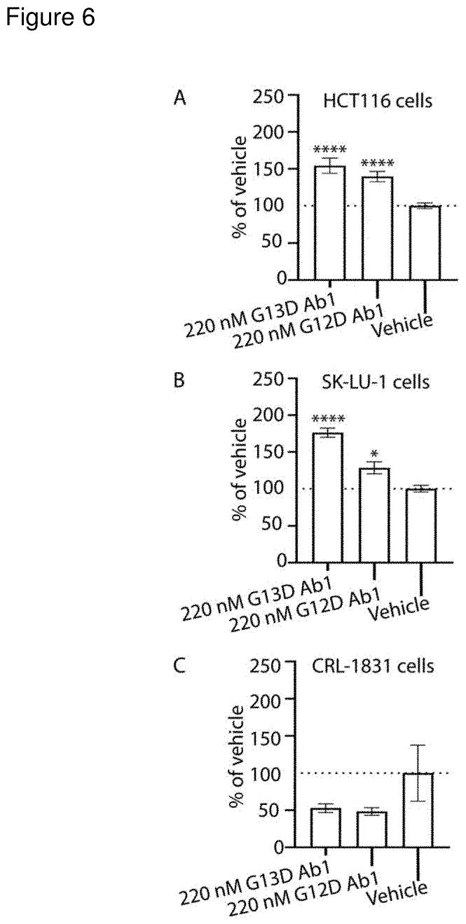

FIG. 6: Determination of necrosis induced by antibodies G13D Ab1 and G12D Ab2 in (A) G13D mutated HCT116 (colon carcinoma) cells; (B) G12D mutated SK-LU-1 (lung adenocarcinoma) cells; and (C) non-cancerous wtKRAS CRL-1831 (colon epithelia) cells. Cells were treated with antibody or control and apoptosis was quantified in real time using RealTime-Glo™ Annexin V Apoptosis and Necrosis Assay kit (Promega). Vehicle is PBS/Tween. This graph represents the state of necrosis at 52 hrs after addition of antibodies/control to the cells. For the HCT116 data, data was collected during 3 days with n=4 per day. For the SK-LU-1 data, data was collected during 2 days with n=4 per day. For the CRL-1831, data was collected during 1 day with n=4. Statistical significance is indicated as follows: *p<0.05, ****p<0.0001. Data is presented as mean±SEM.

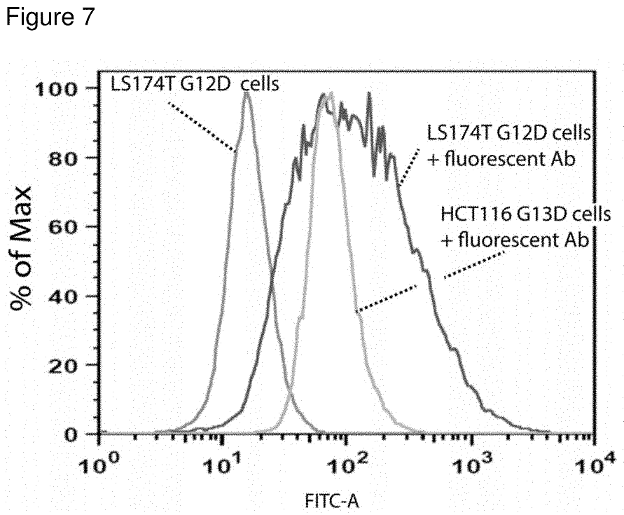

FIG. 7: Uptake of antibodies by cancer cells was quantified by FACS using fluorescence labelled antibody. G12D mutated LS174T (colon adenocarcinoma) cells and G13D mutated HCT116 (colon carcinoma) cells were treated with labelled antibody for 24 hrs and cell-associated fluorescence was quantified. FITC-A=fluorescence signal with emission measured at 488 nm. Area under the curve is proportional to the amount of cell-associated fluorescence. Fluorescence signal from non-antibody treated LS174T cells was used as control. n=1.

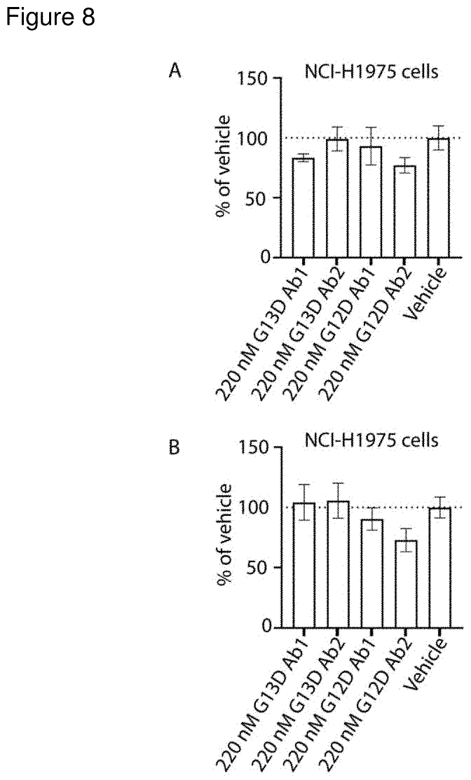

FIG. 8: Determination of apoptosis in wtKRAS NCI-H1975 (lung adenocarcinoma) cells by four anti-KRAS antibodies, G13D Ab1, G13D Ab2, G12D Ab1 and G12D Ab2. Cells were treated with antibody or vehicle. Apoptosis (A) and necrosis (B) were quantified in real time using RealTime-Glo™ Annexin V Apoptosis and Necrosis Assay kit (Promega). (A) represents the state of apoptosis at 10 hrs after addition of antibodies/vehicle to the cells. (B) represents the state of necrosis at 52 hrs after addition of antibodies/vehicle to the cells. Data was collected during a single day, n=4 per treatment. Data is presented as mean±SEM.

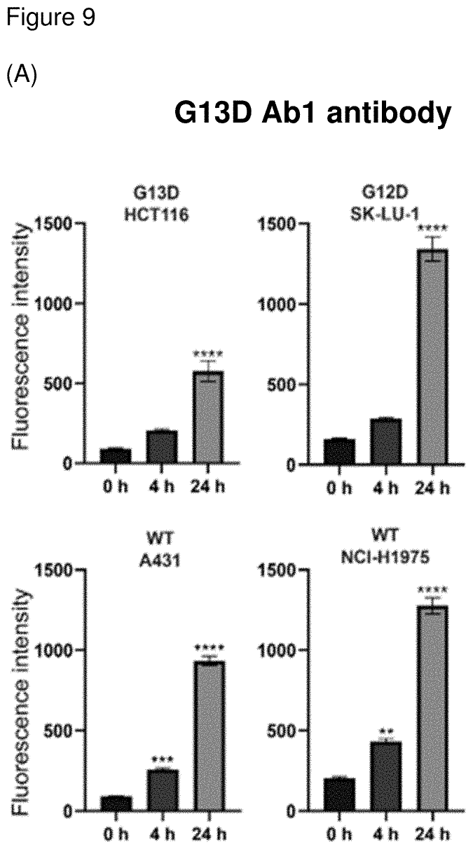

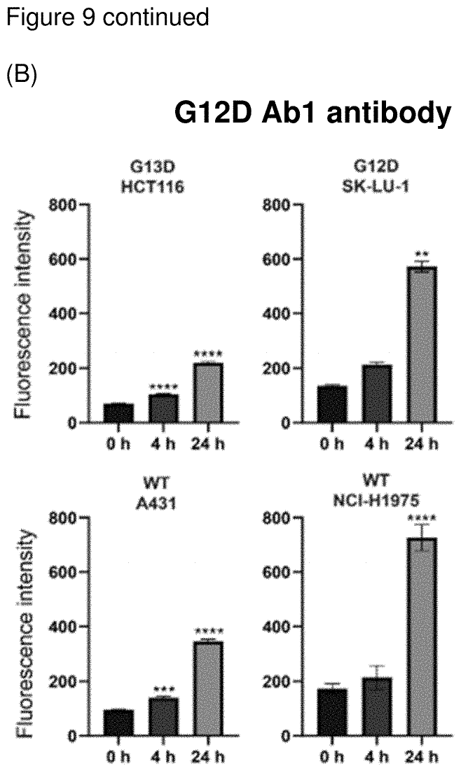

FIG. 9: Flow cytometry demonstrates uptake of antibodies G13D Ab1 and G12D Ab1 in mutated and wild-type KRAS cells. (A) Flow cytometry data showing antibody internalization at different time points after treatment with 220 nM ALEXA 488-conjugated G13D Ab1 antibody in a G13D-mutated KRAS cell line HCT-116, a G12D-mutated KRAS cell line SK-LU-1, and two wild-type KRAS cell lines A431, and NCI-H1975 (n=4). (B) Flow cytometry data showing antibody internalization at different time points after treatment with 220 nM ALEXA 488-conjugated G12D Ab1 antibody in a G13D-mutated KRAS cell line HCT-116, a G12D-mutated KRAS cell line SK-LU-1, and two wild-type KRAS cell lines A431 and, NCI-H1975 (n=4). Statistical significance was determined using a one-way ANOVA in combination with Dunnett's multiple comparisons test. Statistical significance is indicated as follows: **p<0.01, ***p<0.001, ****p<0.0001. Data is presented as mean±SEM.

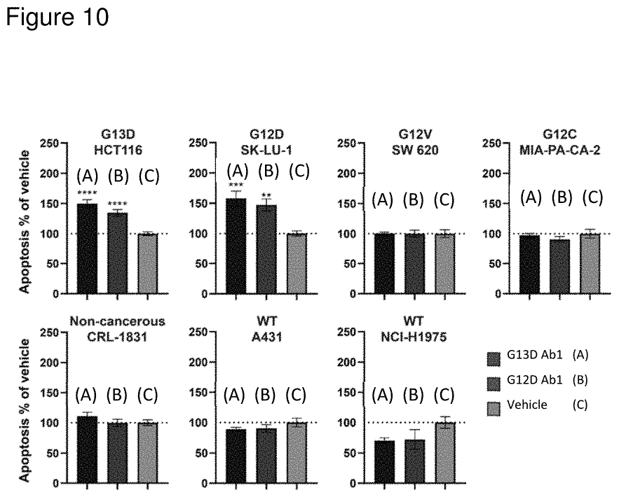

FIG. 10: Evaluation of the apoptosis-inducing capacity of the mutation-selective G13D Ab1 and G12D Ab1 antibodies at 24 hrs in a panel of KRAS-mutated, wtKRAS, and non-cancerous cell lines. The different cell lines investigated include; G13D-mutated HCT116 (colon carcinoma) cells, G12D-mutated SK-LU-1 (lung adenocarcinoma) cells, G12V-mutated SW 620 (colorectal adenocarcinoma) cells, G12C-mutated MIA-PA-CA-2 (pancreatic carcinoma) cells, cancerous wtKRAS A431 (epidermoid carcinoma) cells, cancerous wtKRAS NCI-H1975 (non-small cell lung cancer) cells and non-cancerous wtKRAS CRL-1831 (colon epithelia) cells. Cells were treated with 220 nM antibody or vehicle and apoptosis was quantified in real time using RealTime-Glo™ Annexin V Apoptosis and Necrosis Assay kit (Promega). The graphs represent the level of apoptosis normalized to vehicle, at 24 hrs after addition of antibodies or vehicle to the cell cultures. For HCT116 cells n=10 (G13D Ab1) and 12 (vehicle and G12D Ab1). For SK-LU-1 cells n=8. For SW 620 cells n=8. For MIA-PA-CA-2 cells n=4. For A431 cells n=8. For NCI-H1975 cells n=4. For CRL-1831 cells n=6 (vehicle) and 8 (G13D Ab1 and G12D Ab1). Statistical significance was determined using one-way ANOVA in combination with Dunnett's multiple comparisons test except for SK-LU-1 cells where statistical significance was determined using a Kruskal-Wallis test in combination with Dunn's multiple comparisons test. **p<0.01, ***p<0.001, ****p<0.0001. Data is presented as mean±SEM.

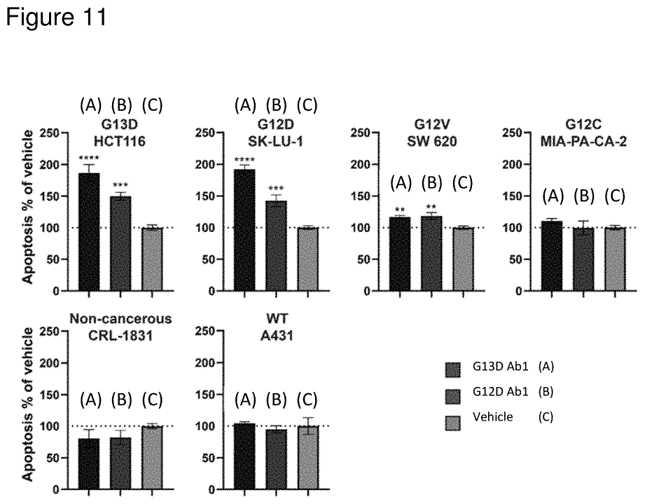

FIG. 11: Evaluation of the apoptosis-inducing capacity of the mutation-selective G13D Ab1 and G12D Ab1 antibodies at 48 hrs in a panel of KRAS-mutated, wtKRAS, and non-cancerous cell lines. The different cell lines investigated include; G13D-mutated HCT116 (colon carcinoma) cells, G12D-mutated SK-LU-1 (lung adenocarcinoma) cells, G12V-mutated SW 620 (colorectal adenocarcinoma) cells, G12C-mutated MIA-PA-CA-2 (pancreatic carcinoma) cells, cancerous wtKRAS A431 (epidermoid carcinoma) cells, and non-cancerous wtKRAS CRL-1831 (colon epithelia) cells. Cells were treated with 220 nM antibody or vehicle and apoptosis was quantified in real time using RealTime-Go™ Annexin V Apoptosis and Necrosis Assay kit (Promega). Cancerous wtKRAS NCI-H1975 cells could not be evaluated at 48 hrs, using the above kit, due to a fast onset of apoptosis. The graphs represent the level of apoptosis normalized to vehicle, at 48 hrs after addition of antibodies or vehicle to the cell cultures. For HCT116 cells n=10 (G13D Ab1) and 12 (vehicle and G12D Ab1). For SK-LU-1 cells n=8. For SW 620 cells n=8. For MIA-PA-CA-2 cells n=4. For A431 cells n=8. For CRL-1831 cells n=6 (vehicle) and 8 (G13D Ab1 and G12D Ab1). Statistical significance was determined using one-way ANOVA in combination with Dunnett's multiple comparisons test except for CRL-183 and MIA-PA-CA-2 cells where statistical significance was determined using a Kruskal-Wallis test in combination with Dunn's multiple comparisons test. **p<0.01, ***p<0.001, ****p<0.0001. Data is presented as mean±SEM.

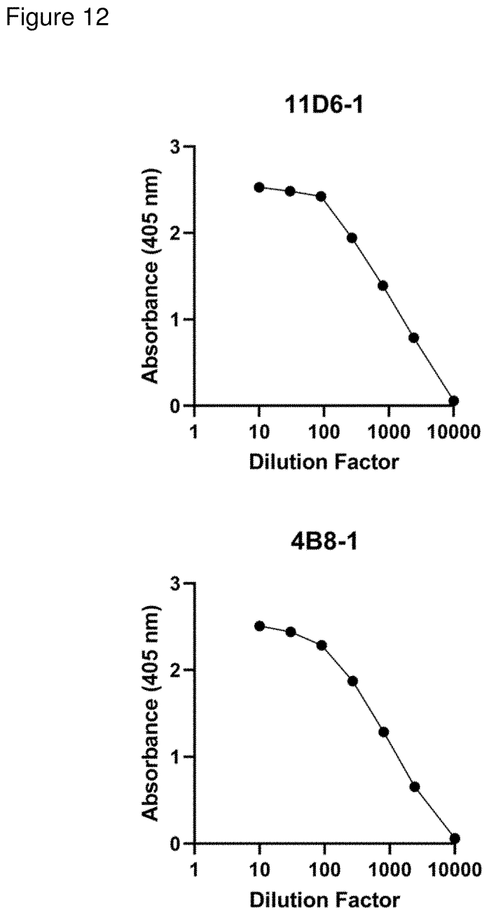

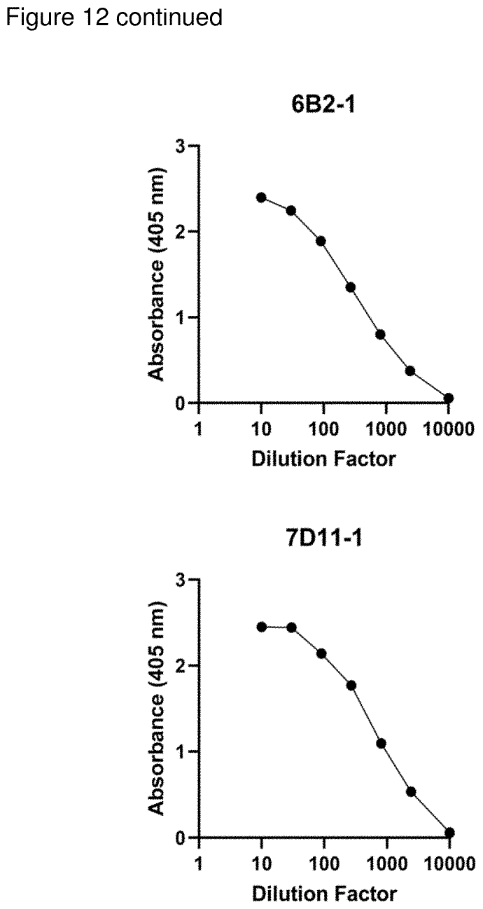

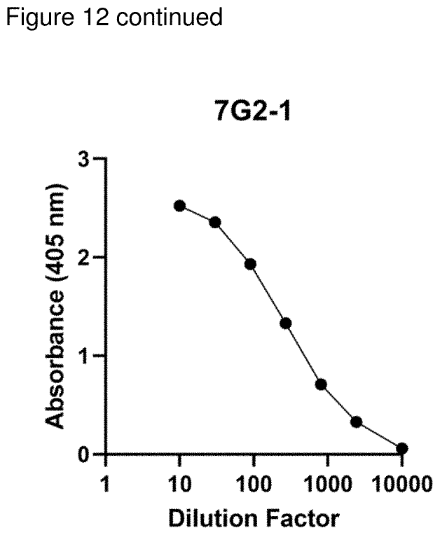

FIG. 12: Monoclonal antibody in hybridoma single cell clone supernatant was tested, using ELISA, for binding to a peptide having the amino acid sequence of SEQ ID NO:5 (which includes the G13D KRAS mutation). Clones 11D6-1, 4B81-1, 6B2-1, 7D11-1, and 7G2-1 were tested at various dilutions of peptide. Absorbance was measured at 405 nm. Higher absorbance indicates stronger binding.

### EXAMPLE 1

### Antibody Development

For each epitope (linear peptide) used for antibody generation, synthetic peptides including an additional cysteine residue, were synthesized and purified. These synthetic peptide sequences are set forth as SEQ ID NOs 3, 4, 5 and 6.

The peptides (epitopes) were linked by the terminal cysteine residue to keyhole limpet hemocyanin (KLH). The KLH-linked peptides (epitopes) were used to produce polyclonal antibodies by immunization of specific pathogen-free (SPF) rabbits following injection of the KLH linked peptides.

The peptides were produced by solid phase peptide synthesis (SPPS) with capping step. Linear peptides were conjugated to KLH by coupling SH-group on cysteine to NH2-group on KLH. Antibodies were purified using a Protein G column followed by affinity purification against the peptide.

The antibodies were affinity purified and subjected to ELISA tests. ELISA tests showed that the antibodies were able to bind to their respective peptides (i.e. the respective peptide used to immunize the rabbit to produce the relevant antibody). ELISA tests also showed that the antibodies were able to bind to the corresponding full-length oncogenic KRAS protein (isoform 2B versions).

Generation of both synthetic peptides and polyclonal antibodies were performed by Innovagen AB (Lund, Sweden).

The polyclonal antibody generated by immunization with the peptide of SEQ ID NO:3 is named G12D Ab1.

The polyclonal antibody generated by immunization with the peptide of SEQ ID NO:4 is named G12D Ab2.

The polyclonal antibody generated by immunization with the peptide of SEQ ID NO:5 is named G13D Ab1.

The polyclonal antibody generated by immunization with the peptide of SEQ ID NO:6 is named G13D Ab2.

### Inhibition of KRAS Functional Activity

### Inhibition of KRAS GTPase Activity

### Materials and Methods

Recombinant wild-type, G12D and G13D forms of KRAS were produced by bacterial expression and purified. Purification tags were removed. KRAS was stored in 50 mM Tris, 100 mM NaCl, 10 mM MgCl₂, 1 mM EDTA, 0.01% Triton-X-100, 1 mM DTT, pH7.5. Batch purity was assessed by gel filtration and SDS-PAGE.

KRAS activity was assessed using a microplate reader (Clariostar, BMG Labtech, Excitation/Emission=430/450). 1 g/L KRAS, 1 μM phosphate sensor (Thermo Fisher Scientific #PV4407), 6 μM GTP and antibody were prepared in buffer H (50 mM Tris, 100 mM NaCl, 10 mM MgCl₂, 1 mM EDTA, 0.01% Triton X-100 and 1 mM DTT) and added to a 384-well plate and fluorescence was measured during 4 h. Fluorescence values after 4 h were compared between treatments.

The G13D Ab2, G12D Ab1 and G12D Ab2 antibodies were tested for their ability to inhibit GTP to GDP conversion (GTPase activity) by wild-type KRAS, G12D mutant KRAS and G13D mutant KRAS. The antibodies preferentially inhibited an oncogenic form of KRAS (G12D or G13D) as compared to wild-type KRAS. No significant inhibition of wild-type KRAS GTPase activity was observed.

### Inhibition of ERK Phosphorylation

### Materials and Methods

MDA-MB-231 cells (from American Type Culture Collection) were plated in 384 well plates at a density of 10000 cells/well and incubated overnight in a 5% CO₂ incubator at 37° C. The next day, cells were treated with compounds (antibodies) or controls (rabbit IgG) in media containing serum and incubated for 14-16 hours in a 5% CO₂ incubator at 37° C. The final working concentrations of compounds (antibodies) were 25 μg/ml (167 nM), 10 μg/ml (67 nM) and 4 μg/ml (27 nM). Rabbit IgG (−ve control) was added at a final concentration of 25 μg/ml (167 nM). The following day, media in each well was replaced with serum-free media containing compounds or controls. The K-Ras inhibitor SAH-SOS1 (stabilized alpha helices of son of sevenless 1, +ve control, 5 μM) was added to respective wells during this 4 h serum starvation period. After serum starvation, cells were treated with 10.9 ng/ml EGF (epidermal growth factor) for 10 min. After EGF treatment, cells were lysed for 10 min with lysis buffer provided in a kit (AlphaScreen® SureFire® p-ERK 1/2 (Thr202/Tyr204) Assay Kits, Perkin Elmer cat no. TGRESB500), which is a sandwich immunoassay for quantitative detection of phospho-ERK1/2 (phosphorylated on Thr202/Tyr204) in cellular lysates. ERK is Extracellular Signal-Regulated Kinase. The lysate was divided and used for measurement of total and phosphorylated ERK. Phosphorylated ERK was estimated following the AlphaScreen® SureFire® kit protocol (Perkin Elmer, cat no. TGRESB500). Total ERK was estimated following a kit protocol (AlphaScreen® SureFire® ERK 1/2 Total Assay Kit, Perkin Elmer cat. no. TGRTESB500), which is a sandwich immunoassay for quantitative detection of ERK (both phosphorylated and non-phosphorylated) in cellular lysates. Readings were taken on the Envision plate reader (Perkin Elmer) using standard AlphaScreen settings. AlphaScreen is a bead-based, non-radioactive Amplified Luminescent Proximity Homogeneous Assay. Statistical significance is indicated as follows: *p<0.05, **p<0.01. Data is presented as mean±SEM.

### Results and Discussion

For antibodies to inhibit KRAS, they must enter the cells and be presented to the cytosol where KRAS resides. The level of phosphorylation of ERK in the MAPK pathway is in many cancer cells dependent on KRAS activity, where ERK is downstream to KRAS (MAPK/ERK pathway). ERK phosphorylation is often used as a measure of KRAS activity when testing anti-KRAS compounds, with inhibition of ERK phosphorylation a result of KRAS inhibition. We measured inhibition of ERK phosphorylation (as % inhibition of phospho-ERK) in the breast cancer cell line MDA-MB-231 by four anti-KRAS antibodies G13D Ab1, G13D Ab2, G12D Ab1 and G12D Ab2. As a positive control we used SAH-SOS1, a known polypeptide KRAS inhibitor that binds to KRAS, and as a negative control we used rabbit IgG which should not bind to KRAS. Antibodies were added to the medium outside the cells and, without wishing to be bound by theory, can enter the cells via the process of macropinocytosis. MDA-MB-231 cells bear a G13D KRAS mutation. We confirmed inhibition of ERK phosphorylation by the antibodies already at an extracellular concentration as low as 27 nM (FIG. 1).

### Apoptosis

### Materials and Methods

For experiments assessing apoptosis in HCT116 cells, SK-LU1 cells and CRL-1831 cells (i.e. the experiments that generated the data depicted in FIGS. 2, 4 and 5), the materials and methods are as follows:

The RealTime-Glo™ Annexin V Apoptosis and Necrosis Assay kit (Promega, cat no. JA1011) was used to measure apoptosis and necrosis in real time. This is a live-cell real-time (kinetic) assay that determines apoptosis by the exposure of phosphatidylserine (PS) on the outer leaflet of the cell membrane and determines necrosis by a cell-impermeant, profluorescent DNA dye that is able to enter cells during the necrotic process as the cell membrane disintegrate.

Cells were prepared as follows. HCT-116, SK-LU-1 and CRL-1831 cells (from American Type Culture Collection) were cultivated in T flasks. HCT-116 cells were grown in McCoy's 5a Medium Modified supplemented with 10% fetal bovine serum. SK-LU-1 cells were grown in Eagle's Minimum Essential Medium supplemented with 10% fetal bovine serum. CRL-1831 cells were grown in DMEM:F12 Medium supplemented with 10% fetal bovine serum, 10 ng/ml cholera toxin, 0.005 mg/ml insulin, 0.005 mg/ml transferrin, 100 ng/ml hydrocortisone and 20 ng/mL human recombinant EGF. Cells were harvested using pre-warmed trypsin solution (37° C.) and incubated for 15 minutes. Cells were transferred to L-15 assay medium, consisting of Leibovitz's L-15 Medium (ThermoFisher cat. no. 21083027), 10% fetal bovine serum and 1% penicillin/streptomycin. Cells were counted and diluted to 200.000 cells/mL with L-15 assay medium. 50 μL of cell solution was added per well in 96 well plates (10.000 cells per well, Costar, white with clear opaque bottom, cat. no. 3903). For measurement of background, wells on the same plate were filled with 50 μL L-15 assay medium. Plates were sealed with adhesive film and incubated overnight at 37° C.

Antibodies were prepared as follows. Antibodies were first mixed into a total of 300 μL of phosphate-buffered saline containing 0.02% Tween (PBS/Tween). The final antibody concentration was 0.87 μM for all antibodies. Working solutions were prepared by mixing antibodies solutions with L-15 assay medium and kit detection reagent (210 μL assay medium+210 μL antibody solution+420 μL detection reagent). Detection reagent was prepared according to the kit instructions. One type of control was made where 210 μL antibody solution was replaced with 210 μL PBS/Tween (vehicle control). Another type of control was made where 210 μL antibody solution was replaced with 210 μL L-15 assay medium (medium control).

Assays were performed as follows. 200 μL of medium was removed from each plate well and replaced with 200 μL of working solution. The plates were the sealed with optic film and placed in a CLARIOstar® (BMG LABTECH) plate reader. Measurement of apoptosis was performed by reading luminescence signal and necrosis by fluorescence signal according to the kit instructions. Data analysis and interpretation was done according to the kit instructions.

Data is presented as mean±SEM. Statistical significance was determined using one-way ANOVA in combination with Dunnett's multiple comparisons test where each Ab (antibody) was compared to isotype control (rabbit IgG) or vehicle control. Statistical significance is indicated as follows: *p<0.05, **p<0.01, ****p<0.0001. Data is presented as mean±SEM.

For experiments assessing apoptosis in LS174T cells (i.e. the experiments that generated the data depicted in FIG. 3), the materials and methods are as follows:

LS174T cells were cultured to 70% confluence in Eagle's Minimum Essential Medium supplemented with 10% fetal bovine serum and 1% Penicillin/Streptomycin. Cells were treated with control (medium without antibody) or 3 ng/ml antibody in medium for 24, 48 or 72 hours. Cells were then washed and stained with fluorescence labelled Annexin V Pacific Blue (ThermoFisher, cat no. A35122) which binds to phosphatidylserine (PS) that becomes exposed on the outer leaflet of cells undergoing apoptosis. FACS was used for quantification of cell-associated fluorescence, and the level of apoptosis (% of control) was calculated as the ratio of fluorescence between antibody-treated cells and control cells.

### Results and Discussion

In some experiments, apoptosis was quantified using a real-time apoptosis/necrosis kit from Promega, which measures the amount of phosphoserine lipid (PS) at the external side of the cellular membrane. PS amount increases during the apoptotic process. We have determined that the anti-KRAS antibodies here, G13D Ab1, G13D Ab2, G12D Ab1 and G12D Ab2, induce apoptosis in G12D mutated SK-LU-1 (lung adenocarcinoma) cells and/or G13D mutated HCT116 (colon carcinoma) cells (FIGS. 2, 4 and 5). Cells were treated with antibody by adding antibody to the cell medium. The data in FIG. 3 shows that the antibody G12D Ab1 is also able to induce apoptosis in LS174T (G12D) cells.

G13D Ab1 and G12D Ab1 caused high levels of apoptosis in both SK-LU-1 cells (FIG. 2) and HCT116 cells (FIG. 4) compared to isotype antibody control, or compared to vehicle PBS/Tween (FIG. 5A,B). This data indicates that both of these antibodies are active against both mutations (i.e. G12D and G13D mutant forms of KRAS). Without wishing to be bound by theory, it is believed that these antibodies may bind to the negatively charged aspartic acid (D) residue but that the antibodies can accommodate the D residue at either position 12 or 13 due to the small difference in distance. Some apoptosis was observed in CRL-1831 colon epithelia cells which have wild-type KRAS, although compared to cells harbouring mutated KRAS it was much less for both antibodies tested (FIG. 5C).

Apoptosis induced by KRAS antibodies of the present study shows that the synthetic antigens (which correspond essentially to amino acid residues 10-21 of oncogenic mutant forms of KRAS as described elsewhere herein) used for immunization and antibody production can be used to develop inhibitory KRAS antibodies. Furthermore, the data shows that antibodies can be generated which can inhibit both G12D and G13D mutant forms of KRAS.

### Necrosis

### Materials and Methods

Necrosis was recorded in parallel to apoptosis using the RealTime-Glo™ Annexin V Apoptosis and Necrosis Assay kit assay kit as described elsewhere.

### Results and Discussion

It has been shown herein that, in addition to apoptosis, the anti-KRAS antibodies G13D Ab1 and G12D Ab1 caused significant necrosis in SK-LU-1 cells (FIG. 6A) and HCT116 cells (FIG. 6B) but not wild-type KRAS cells CRL-1831 (FIG. 6C).

### Further Apoptosis and Necrosis Experiment

The effect of the antibodies G12D Ab1, G12D Ab2, G13D Ab1 and G13D Ab2 on apoptosis and necrosis of the lung adenocarcinoma cell line NCI-H1975 was tested. The NCI-H1975 cell line expresses wild-type KRAS. The materials and methods for this experiment are essentially as per the apoptosis and necrosis assays described above (the RealTime-Glo™ Annexin V Apoptosis and Necrosis Assay kit (Promega) was used).

In this experiment, the level of apoptosis and necrosis of NCI-H1975 cells treated with each of antibodies G12D Ab1, G12D Ab2, G13D Ab1 and G13D Ab2 was similar to, or less than, the level of apoptosis and necrosis observed with the vehicle-only control (FIG. 8).

### Antibody Internalization

Materials and Methods Cells (LS174T cells or HCT116 cells) were cultured to 70% confluence in Eagle's Minimum Essential Medium supplemented with 10% fetal bovine serum and 1% Penicillin/Streptomycin. Cells were treated with 16 ng/ml rabbit anti-Goat IgG (H+L) Secondary Antibody, Alexa Fluor 488 (ThermoFisher, cat. no. A-11078) dissolved in the same medium for 24 hrs. Cells were washed and cell-associated fluorescence was quantified using FACS.

### Results and Discussion

In order to assess whether cancer cells can internalize antibodies presented in the extracellular medium we used fluorescence-tagged antibodies and quantified uptake quantified by FACS. G12D mutated LS174T (colon adenocarcinoma) cells and G13D mutated HCT116 (colon carcinoma) cells were treated with labelled antibody for a duration, and cellular fluorescence was quantified. Based on the area under the curve of the accumulated florescence signal in treated cells, compared to the signal from untreated cells, we drew the conclusion that there exists a mechanism of uptake of antibody in these cancer cell types.

### EXAMPLE 2

This Example shows that antibodies of the invention are internalized by cancer cells, including cancer cells expressing oncogenic mutant forms of KRAS. This Example also contains further data showing that antibodies of the invention cause apoptosis in cells expressing oncogenic mutant forms of KRAS.

### Materials and Methods

### Flow Cytometry Measurements of G13D Ab1 Antibody and G12D Ab1 Antibody Uptake

Antibody G13D Ab1 and antibody G12D Ab1 (of Example 1) were labelled with Alexa Fluor 488 using an Alexa Fluor 488 protein labeling kit (Cat # A10235, ThermoFisher Scientific). HCT 116, SK-LU-1, A431 and NCI-H1975 cells were cultured in 12-well microplates (Invitrogen). Cells were treated with 220 nM labelled G13D Ab1 antibody or G12D Ab1 antibody for 4 or 24 hours prior to measurements. Unstained cells were measured as a t(0) timepoint. Prior to measurements, cells were washed and harvested. Measurements were performed on a BD™ LSR II flow cytometer (BD Biosciences inc.). Analysis was performed in FlowJo v.10 (BD Biosciences). For each cell line and time-point n=4. n equals one measurement containing on average 4000 cells for HCT116, 8000 cells for SK-LU-1, 7000 cells for A431 and 4000 cells for NCI-H1975.

Antibody Induced Apoptosis of aKRA S Antibodies (G13D Ab1 Antibody and G12D Ab1 Antibody)

The RealTime-Glo™ Annexin V Apoptosis and Necrosis Assay kit (Promega, cat no. JA1011) was used to measure apoptosis in real time. Cultured cells were harvested using trypsin and transferred to L-15 assay medium, consisting of Leibovitz's L-15 Medium (ThermoFisher cat. no. 21083027), 10% fetal bovine serum and 1% penicillin/streptomycin. Cells were counted and diluted to 200,000 cells/mL with L-15 assay medium. Cells were added in 96 well plates (10,000 cells per well, Costar, white with clear opaque bottom, cat. no. 3903) and incubated overnight.

Antibodies were diluted in L-15 assay medium and the detection reagent and added to the wells in a final concentration of 220 nM. The plates were the sealed with optic film and placed in a CLARIOstar® (BMG LABTECH) plate reader. Measurement of apoptosis was performed by reading of the luminescence signal.

2-3 biological replicates were performed for the following cell lines: HCT 116, SK LU 1, SW 620, CRL-1831 and A431. The NCI-H1975 and MIA-PA-CA-2 cell lines were tested once.

### Results and Discussion

The in vitro anti-cancer efficacy of the G13D Ab1 and G12D Ab1 antibodies was determined by evaluating their capability to cause apoptosis in four different KRAS-mutated cell lines, i.e. G12D-mutated SK-LU-1 lung adenocarcinoma cells, G13D-mutated HCT116 colon carcinoma cells, G12V-mutated SW 620 colorectal adenocarcinoma cells, G12C-mutated MIA-PA-CA pancreatic carcinoma cells, as well as wild-type KRAS cell lines, i.e. A431 epidermoid carcinoma cells, and NCI-H1975 non-small cell lung cancer cells. A non-cancerous cell line CRL-1831 colon epithelial cells was also included as a control cell line in the apoptosis experiments. Although KRAS is an intracellular target, we exploited the fact that many cancer cell lines, including KRAS-mutated cells, have upregulated macropinocytosis. This uptake system has a well-documented capacity to internalize macromolecules, including proteins. We used flow cytometry and confocal microscopy experiments to evaluate internalization of Alexa Fluor 488-labelled G13D Ab1 and G12D Ab1 antibodies in the cell line bearing the G12D mutation (SK-LU-1) and the cell line bearing the G13D mutation (HCT116) as well as in the two wild-type KRAS cell lines A431, and NCI-H1975. The confocal microscopy images showed uptake of the G13D Ab1 and G12D Ab1 antibodies in all the different cell lines examined (data not shown). Flow cytometry experiments further confirmed these observations, and a statistically significant antibody uptake was observed in all tested KRAS-mutated and all tested wild-type KRAS cell lines as shown in FIG. 9. In the apoptosis experiments, the G13D Ab1 and G12D Ab1 antibodies were active only in G12D- and G13D-mutated cell lines after 24 hrs of treatment (FIG. 10). The antibodies did not cause apoptosis in any of the other cell lines tested which included G12C and G12V KRAS-mutated cells, wild type KRAS cancer cells and non-cancerous colon epithelial cells (FIG. 10). After 48 hrs of treatment (FIG. 11), we observed that the G13D Ab1 and G12D Ab1 antibodies elicited an increased level of apoptosis in G12D- and G13D-mutated cell lines, compared to the 24 h treatment. We also observed a small, but stastistically significant, increase of apoptosis in G12V-mutated cells after 48 h (FIG. 11). None of the other cell lines showed any increase in apoptosis after 48 h of antibody exposure (FIG. 11). Taken together, the data show that the apoptosis-inducing effect in G12D- and G13D-mutated cells compared to wild-type KRAS cells, is not a result of a quantitatively differential antibody uptake in favor of KRAS-mutated cells, but rather a result from a selective inhibition of G12D and G13D-mutated KRAS cells by the G13D Ab1 and G12D Ab1 antibodies.

The selective effects of the antibodies on G12D and G13D mutants but not on G12C and to a much lesser extent on G12V mutants may be explained by that the negatively charged aspartic acid residue (D) at the G12 and G13 position in the epitope region plays an important role in the binding of the antibodies. However, the allele location at either position 12 or 13, due to the small difference in distance, seems to be of minor importance for the binding interaction.

This study demonstrates two anti-KRAS antibodies that are capable of inhibiting G12D- and G13D-mutated KRAS with selectivity over other KRAS mutations (G12V and G12C) as well as over wild-type KRAS.

### EXAMPLE 3

### Materials and Methods

### Production of Monoclonal Antibodies (Mouse IgG) Using the Hybridoma Technology

Mice were immunized with the linear peptide of SEQ ID NO:5 linked to keyhole limpet hemocyanin (the KLH linked peptide is as described above in Example 1). The “D” residue at residue number (position) 5 of SEQ ID NO:5 corresponds to the oncogenic G13D mutation of an oncogenic mutant G13D KRAS protein (e.g. an oncogenic mutant G13D KRAS protein of SEQ ID NO:9 or SEQ ID NO:12). Immune responses were evaluated with ELISA. After the immunization process mice were selected based on ELISA screening of serum, and spleen cells from mouse were extracted and fused with myeloma cells to produce hybridoma cells. Hybridomas were screened by ELISA to obtain positive clones (i.e. that produce antibody that binds to the target). After this screening, sub-cloning of the selected hybridomas was performed and a further round of screening was performed using ELISA. Sub-cloned hybridomas were then used to produce monoclonal antibodies. The binding properties of antibodies in the supernatant were tested using ELISA. Five monoclonal antibodies (hybridomas) were identified, 11D6-1, 4B8-1, 6B2-1, 7D11-1, 7G2-1. The peptide (immunogen) used to generate the 11D6-1, 4B8-1, 6B2-1, 7D11-1, 7G2-1 antibodies was the peptide of SEQ ID NO:5, and as mentioned above this was linked to keyhole limpet hemocyanin. The suffix “−1” in each of the antibody names is merely indicative that each of these antibodies is a daughter clone of a respective parental hybridoma (the parental hybridomas have the same names without the “−1” suffix, for example 11D6 is the parental hybridoma of the 11D6-1 antibody (daughter clone)).

### Cloning and Sequencing of Mouse Hybridoma IgG

From mouse hybridomas (11D6-1, 4B8-1, 6B2-1, 7D11-1 and 7G2-1), RNA was prepared from which cDNA was synthesized. Variable Light (VL) and Variable Heavy (VH) regions of cDNA were amplified and cloned into standard cloning vector separately. Identification of positive clones was done by colony PCR followed by gel electrophoresis. VL and VH DNA and amino acid sequences were obtained from positive clones.

Sequencing of 6B2-1 identified one VH region (domain) sequence, but two different VL region (domain) sequences (i.e. two distinct VL region sequences were identified). This is discussed further below in the results section.

Sequencing of the cDNAs encoding the VH and VL domains of the other four antibodies (i.e. 11D6-1, 4B8-1, 7D11-1 and 7G2-1) identified one VH region (domain) sequence and one VL region (domain) sequence per antibody (i.e. per hybridoma).

### Results and Discussion

Monoclonal antibody in hybridoma supernatant preparations from hybridoma clones 11D6-1, 4B8-1, 6B2-1, 7D11-1 and 7G2-1 were tested by ELISA for binding to various dilutions of the peptide having the amino acid sequence of SEQ ID NO:5 (which was peptide (immunogen) used to generate the 11D6-1, 4B8-1, 6B2-1, 7D11-1 and 7G2-1 antibodies). Quantitation of antibody bound to peptide was measured by absorbance at 405 nm. Absorbance is proportional to binding strength. All supernatants contained antibody that bound to the peptide of SEQ ID NO:5 (FIG. 12). A further ELISA performed using antibodies from clones 11D6-1, 4B8-1 and 7D11-1 and using a “wild-type” (WT) peptide (having the amino acid sequence CGAGGVGKSALTI (SEQ ID NO:160)) that has a G residue instead of a “mutant” D residue at position 5 (as compared to SEQ ID NO:5) showed that antibodies from clones 11D6-1, 4B8-1, and 7D11-1 clearly bound more strongly to the mutant G13D peptide (SEQ ID NO:5) than to the WT peptide (data not shown). If an antibody binds more strongly to a mutant-containing peptide than the WT peptide it is indicative that the antibody is selective for the mutant form of KRAS, that it has a preference for the mutated sequence. Without wishing to be bound by theory, such selective antibodies prefer to bind to a disease-causing mutant form of the KRAS protein. Such a preference may be important in a case when a cell or tissue is expressing not only the mutated form but also the wild-type form of the KRAS protein.

As mentioned above, sequencing of VH and VL regions (domains) of the 6B2-1 hybridoma identified one VH region (domain) sequence, but two different (i.e. distinct) VL region (domain) sequences. This could mean that hybridoma clone 6B2-1 has one heavy chain gene and two light chain genes that are producing antibodies. This phenomenon (i.e. of a hybridoma encoding two distinct VL domain sequences) is known to occur and has been discussed in the literature (e.g. Bradbury, A. R. M. et al. MAbs 10, 539-546 (2018)). A situation where there is one VH region (domain) sequence, but two different VL region (domain) sequences can result potentially in three different species of monoclonal antibodies ((i) a species that has two identical heavy chains and two identical light chains of sequence X; (ii) a species that has two identical heavy chains and two identical light chains of sequence Y; (iii) a species that has two identical heavy chains, one light chain of sequence X and one light chain of sequence Y). The supernatant (or purified antibody) from this hybridoma (6B2-1) might contain a mixture of one or more of these types of species. In normal circumstances only one heavy chain gene and one light chain gene are producing antibody. Hybridomas containing more than one light chain can arise during hybridoma generation and cultivation. There can be a number of causes. For example, a myeloma cell may fuse two B cells instead of only one. The tested 6B2-1 supernatant in this Example could contain one or more of the three potential species of antibody.

Elsewhere herein, the sequences of two potential monoclonal antibodies (two potential monoclonal antibody species) from the 6B2-1 hybridoma are set forth separately. These are (a) an antibody that has the identified VH domain sequence and only the first identified VL domain sequence, and (b) an antibody that has the identified VH domain sequence and only the second (or other) identified VL domain sequence. These distinct monoclonal antibodies (distinct monoclonal antibody species) have been given the names 6B2-1-1 and 6B2-1-2.

Sequences of the antibodies 11D6-1, 4B8-1, 7D11-1, 7G2-1, 6B2-1-1 and 6B2-1-2, are set out in Tables A-F herein.

1. An antibody which binds to an oncogenic mutant form of KRAS, said oncogenic mutant form of KRAS comprising an amino acid substitution at the position corresponding to position G13 or G12 of wild-type KRAS (SEQ ID NO:7), wherein said antibody binds to an epitope that is in the region of said oncogenic mutant form of KRAS that is defined by the amino acid residues that correspond to amino acid residues 10-21 of wild-type KRAS (SEQ ID NO:7), and wherein said antibody inhibits activity of said oncogenic mutant form of KRAS.

2. The antibody of claim 1, wherein said oncogenic mutant form of KRAS has the amino acid sequence of SEQ ID NO:14, 16, 13 or 15 and wherein said antibody binds to an epitope that is in the region of said oncogenic mutant form of KRAS that is defined by amino acid residues 10-21 of SEQ ID NO:14, 16, 13 or 15.

3. The antibody of claim 1 or claim 2, wherein said amino acid substitution at the position corresponding to position G13 or G12 of wild-type KRAS is a G13D or a G12D substitution.

4. The antibody of any one of claims 1 to 3, wherein said oncogenic mutant form of KRAS comprises an amino acid substitution at the position corresponding to position G13 of wild-type KRAS (SEQ ID NO:7) and said amino acid substitution is a G13D substitution.

5. The antibody of any one of claims 1 to 4, wherein said oncogenic mutant form of KRAS has the amino acid sequence of SEQ ID NO: 9, 12, 8 or 11 and wherein said antibody binds to an epitope that is in the region of said oncogenic mutant form of KRAS that is defined by amino acid residues 10-21 of SEQ ID NO: 9, 12, 8 or 11.

6. The antibody of any one of claims 1 to 5, wherein said oncogenic mutant form of KRAS has the amino acid sequence of SEQ ID NO:9 or SEQ ID NO: 12 and wherein said antibody binds to an epitope that is in the region of said oncogenic mutant form of KRAS that is defined by amino acid residues 10-21 of SEQ ID NO:9 or SEQ ID NO:12.

7. The antibody of any one of claims 1 to 6, wherein said oncogenic mutant form of KRAS has the amino acid sequence of SEQ ID NO:8 or SEQ ID NO: 11 and wherein said antibody binds to an epitope that is in the region of said oncogenic mutant form of KRAS that is defined by amino acid residues 10-21 of SEQ ID NO:8 or SEQ ID NO:11.

8. The antibody of any one of claims 1 to 7, wherein said antibody binds to and inhibits at least one oncogenic mutant form of KRAS having an amino acid sequence of SEQ ID NO:8 or 11 and to at least one oncogenic mutant form of KRAS having an amino acid sequence of SEQ ID NO:9 or 12.

9. The antibody of any one of claims 1 to 8, wherein said antibody binds to an isolated peptide, said isolated peptide comprising
(i) an amino acid sequence of SEQ ID NO:18 or SEQ ID NO:17; or
(ii) an amino acid sequence substantially homologous to an amino acid sequence of (i), wherein said substantially homologous sequence has 1 or 2 amino acid substitutions or additions or deletions compared with said amino acid sequence and wherein in a sequence substantially homologous to SEQ ID NO:17 the X residue corresponding to position 3 of SEQ ID NO:17 is not altered and wherein in a sequence substantially homologous to SEQ ID NO:18 the X residue corresponding to position 4 of SEQ ID NO:18 is not altered.

10. The antibody of any one of claims 1 to 9, wherein said antibody binds to an isolated peptide, said isolated peptide comprising an amino acid sequence of SEQ ID NO:18 or SEQ ID NO:17.

11. The antibody of any one of claims 1 to 10, wherein said antibody binds to an isolated peptide, said isolated peptide comprising
(i) an amino acid sequence of SEQ ID NO:2 or SEQ ID NO:1; or
(ii) an amino acid sequence substantially homologous to an amino acid sequence of (i), wherein said substantially homologous sequence has 1 or 2 amino acid substitutions or additions or deletions compared with said amino acid sequence and wherein in a sequence substantially homologous to SEQ ID NO:1 the D residue corresponding to position 3 of SEQ ID NO:1 is not altered and wherein in a sequence substantially homologous to SEQ ID NO:2 the D residue corresponding to position 4 of SEQ ID NO:2 is not altered.

12. The antibody of any one of claims 1 to 11, wherein said antibody binds to an isolated peptide, said isolated peptide comprising an amino acid sequence of SEQ ID NO:2 or SEQ ID NO:1.

13. The antibody of any one of claims 1 to 12, wherein said antibody binds to an isolated peptide, said isolated peptide comprising
(i) an amino acid sequence of SEQ ID NO:5, SEQ ID NO:3, SEQ ID NO:4 or SEQ ID NO:6; or
(ii) an amino acid sequence substantially homologous to an amino acid sequence of (i), wherein said substantially homologous sequence has 1 or 2 amino acid substitutions or additions or deletions compared with said amino acid sequence and wherein in a sequence substantially homologous to SEQ ID NO:3 the D residue corresponding to position 3 of SEQ ID NO:3 is not altered and wherein in a sequence substantially homologous to SEQ ID NO:4 the D residue corresponding to position 4 of SEQ ID NO:4 is not altered and wherein in a sequence substantially homologous to SEQ ID NO:5 the D residue corresponding to position 5 of SEQ ID NO:5 is not altered and wherein in a sequence substantially homologous to SEQ ID NO:6 the D residue corresponding to position 4 of SEQ ID NO:6 is not altered.

14. The antibody of any one of claims 1 to 13, wherein said antibody binds to an isolated peptide, said isolated peptide comprising an amino acid sequence of SEQ ID NO:5, SEQ ID NO:3, SEQ ID NO:4 or SEQ ID NO:6.

15. The antibody of any one of claims 1 to 14, wherein said antibody binds to an isolated peptide, said isolated peptide consisting of an amino acid sequence selected from the group consisting of: SEQ ID NO:5, SEQ ID NO:3, SEQ ID NO:4 and SEQ ID NO:6.

16. The antibody of any one of claims 1 to 15, wherein said antibody binds to an isolated peptide, said isolated peptide consisting of an amino acid sequence selected from the group consisting of SEQ ID NO:5 and SEQ ID NO:3.

17. The antibody of any one of claims 1 to 16, wherein said antibody binds to an isolated peptide, said isolated peptide consisting of an amino acid sequence of SEQ ID NO:5.

18. The antibody of any one of claims 1 to 17, wherein said antibody binds to a conjugate, said conjugate comprising an isolated peptide as defined in any one of claims 9 to 17 and a peptide carrier, preferably said peptide carrier is keyhole limpet hemocyanin (KLH).

19. The antibody of any one of claims 1 to 18, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:148 preferably SEQ ID NO: 149, and/or a VH CDR2 that has the amino acid sequence of SEQ ID NO:150 preferably SEQ ID NO: 151, and/or said light chain variable region comprises a VL CDR1 that has the amino acid sequence of SEQ ID NO:152 preferably SEQ ID NO: 153, and/or a VL CDR2 that has the amino acid sequence of SEQ ID NO:154 preferably SEQ ID NO:155.

20. The antibody of any one of claims 1 to 19, wherein said antibody comprises at least one heavy chain variable region that comprises three CDRs and at least one light chain variable region that comprises three CDRs, wherein said light chain variable region comprises a variable light (VL) CDR3 that has the amino acid sequence of SEQ ID NO:37 or a sequence substantially homologous thereto,
wherein said substantially homologous sequence is a sequence containing 1, 2 or 3 amino acid substitutions compared to the given CDR sequence, or said substantially homologous sequence is a sequence containing conservative amino acid substitutions of the given CDR sequence.

21. The antibody of any one of claims 1 to 20, wherein said antibody comprises at least one heavy chain variable region that comprises three CDRs and at least one light chain variable region that comprises three CDRs, wherein said heavy chain variable region comprises a variable heavy (VH) CDR3 that has the amino acid sequence of SEQ ID NO:34 or SEQ ID NO:74, or a sequence substantially homologous thereto,
wherein said substantially homologous sequence is a sequence containing 1, 2 or 3 amino acid substitutions compared to the given CDR sequences, or said substantially homologous sequence is a sequence containing conservative amino acid substitutions of the given CDR sequences.

22. The antibody of any one of claims 1 to 21, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:32, a VH CDR2 that has the amino acid sequence of SEQ ID NO:33 and a VH CDR3 that has the amino acid sequence of SEQ ID NO:34, or sequences substantially homologous thereto, and/or wherein said light chain variable region comprises a variable light (VL) CDR1 that has the amino acid sequence of SEQ ID NO:35, a VL CDR2 that has the amino acid sequence of SEQ ID NO:36 and a VL CDR3 that has the amino acid sequence of SEQ ID NO:37, or sequences substantially homologous thereto,
wherein said substantially homologous sequences are sequences containing 1, 2 or 3 amino acid substitutions compared to the given CDR sequences, or said substantially homologous sequence is a sequence containing conservative amino acid substitutions of the given CDR sequences.

23. The antibody of any one of claims 1 to 22, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:32, a VH CDR2 that has the amino acid sequence of SEQ ID NO:33 and a VH CDR3 that has the amino acid sequence of SEQ ID NO:34 and wherein said light chain variable region comprises a variable light (VL) CDR1 that has the amino acid sequence of SEQ ID NO:35, a VL CDR2 that has the amino acid sequence of SEQ ID NO:36 and a VL CDR3 that has the amino acid sequence of SEQ ID NO:37.

24. The antibody of any one of claims 1 to 21, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:52, a VH CDR2 that has the amino acid sequence of SEQ ID NO:53 and a VH CDR3 that has the amino acid sequence of SEQ ID NO:54, or sequences substantially homologous thereto, and/or wherein said light chain variable region comprises a variable light (VL) CDR1 that has the amino acid sequence of SEQ ID NO:55, a VL CDR2 that has the amino acid sequence of SEQ ID NO:56 and a VL CDR3 that has the amino acid sequence of SEQ ID NO:57, or sequences substantially homologous thereto,
wherein said substantially homologous sequences are sequences containing 1, 2 or 3 amino acid substitutions compared to the given CDR sequences, or said substantially homologous sequence is a sequence containing conservative amino acid substitutions of the given CDR sequences.

25. The antibody of any one of claims 1 to 21 or claim 24, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:52, a VH CDR2 that has the amino acid sequence of SEQ ID NO:53 and a VH CDR3 that has the amino acid sequence of SEQ ID NO:54 and wherein said light chain variable region comprises a variable light (VL) CDR1 that has the amino acid sequence of SEQ ID NO:55, a VL CDR2 that has the amino acid sequence of SEQ ID NO:56 and a VL CDR3 that has the amino acid sequence of SEQ ID NO:57.

26. The antibody of any one of claims 1 to 21, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:72, a VH CDR2 that has the amino acid sequence of SEQ ID NO:73 and a VH CDR3 that has the amino acid sequence of SEQ ID NO:74, or sequences substantially homologous thereto, and/or wherein said light chain variable region comprises a variable light (VL) CDR1 that has the amino acid sequence of SEQ ID NO:75, a VL CDR2 that has the amino acid sequence of SEQ ID NO:76 and a VL CDR3 that has the amino acid sequence of SEQ ID NO:77, or sequences substantially homologous thereto,
wherein said substantially homologous sequences are sequences containing 1, 2 or 3 amino acid substitutions compared to the given CDR sequences, or said substantially homologous sequence is a sequence containing conservative amino acid substitutions of the given CDR sequences.

27. The antibody of any one of claims 1 to 21 or claim 26, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:72, a VH CDR2 that has the amino acid sequence of SEQ ID NO:73 and a VH CDR3 that has the amino acid sequence of SEQ ID NO:74 and wherein said light chain variable region comprises a variable light (VL) CDR1 that has the amino acid sequence of SEQ ID NO:75, a VL CDR2 that has the amino acid sequence of SEQ ID NO:76 and a VL CDR3 that has the amino acid sequence of SEQ ID NO:77.

28. The antibody of any one of claims 1 to 27, wherein said antibody comprises at least one heavy chain variable region that comprises three CDRs and at least one light chain variable region that comprises three CDRs, wherein
(i) the light chain variable region has the amino acid sequence of SEQ ID NO:31, or a sequence having at least 80% sequence identity thereto and/or wherein the heavy chain variable region has the amino acid sequence of SEQ ID NO:30, or a sequence having at least 80% sequence identity thereto;
(ii) the light chain variable region has the amino acid sequence of SEQ ID NO:51, or a sequence having at least 80% sequence identity thereto and/or wherein the heavy chain variable region has the amino acid sequence of SEQ ID NO:50, or a sequence having at least 80% sequence identity thereto; or
(iii) the light chain variable region has the amino acid sequence of SEQ ID NO:71, or a sequence having at least 80% sequence identity thereto and/or wherein the heavy chain variable region has the amino acid sequence of SEQ ID NO:70, or a sequence having at least 80% sequence identity thereto.

29. The antibody of any one of claims 1 to 18, wherein said antibody comprises at least one heavy chain variable region that comprises three CDRs and at least one light chain variable region that comprises three CDRs, wherein heavy chain variable region comprises a variable heavy (VH) CDR1 that has the amino acid sequence of SEQ ID NO:156 preferably SEQ ID NO: 157, and/or a VH CDR2 that has the amino acid sequence of SEQ ID NO:158 preferably SEQ ID NO: 159.

30. The antibody of any one of claims 1 to 18 or claim 29, wherein said antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:156 or preferably SEQ ID NO:157, or a sequence substantially homologous thereto,
(b) a VH CDR2 that has the amino acid sequence of SEQ ID NO:158 or preferably SEQ ID NO:159, or a sequence substantially homologous thereto, and
(c) a VH CDR3 that has the amino acid sequence of SEQ ID NO:94 or SEQ ID NO:114 or SEQ ID NO:134, or a sequence substantially homologous thereto; and/or
wherein said light chain variable region comprises:
(d) a variable light (VL) CDR1 that has the amino acid sequence of SEQ ID NO:95 or SEQ ID NO:115 or SEQ ID NO:135, or a sequence substantially homologous thereto,
(e) a VL CDR2 that has the amino acid sequence of SEQ ID NO:96 or SEQ ID NO:116 or SEQ ID NO:136, or a sequence substantially homologous thereto, and
(f) a VL CDR3 that has the amino acid sequence of SEQ ID NO:97, SEQ ID NO:117 or SEQ ID NO:137, or a sequence substantially homologous thereto,
wherein said substantially homologous sequence is a sequence containing 1, 2 or 3 amino acid substitutions compared to the given CDR sequence, or said substantially homologous sequence is a sequence containing conservative amino acid substitutions of the given CDR sequence.

31. The antibody of any one of claims 1 to 18, 29 or 30, wherein said antibody comprises at least one heavy chain variable region that comprises three CDRs and at least one light chain variable region that comprises three CDRs, wherein light chain variable region comprises a variable light (VL) CDR2 that has the amino acid sequence of SEQ ID NO:96, or a sequence substantially homologous thereto,
wherein said substantially homologous sequence is a sequence containing 1, 2 or 3 amino acid substitutions compared to the given CDR sequence, or said substantially homologous sequence is a sequence containing conservative amino acid substitutions of the given CDR sequence.

32. The antibody of any one of claims 1 to 18, 29, 30 or 31, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:92, a VH CDR2 that has the amino acid sequence of SEQ ID NO:93 and a VH CDR3 that has the amino acid sequence of SEQ ID NO:94, or sequences substantially homologous thereto, and/or wherein said light chain variable region comprises a variable light (VL) CDR1 that has the amino acid sequence of SEQ ID NO:95, a VL CDR2 that has the amino acid sequence of SEQ ID NO:96 and a VL CDR3 that has the amino acid sequence of SEQ ID NO:97, or sequences substantially homologous thereto,
wherein said substantially homologous sequences are sequences containing 1, 2 or 3 amino acid substitutions compared to the given CDR sequences, or said substantially homologous sequence is a sequence containing conservative amino acid substitutions of the given CDR sequences.

33. The antibody of any one of claims 1 to 18, 29, 30, 31 or 32, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:92, a VH CDR2 that has the amino acid sequence of SEQ ID NO:93 and a VH CDR3 that has the amino acid sequence of SEQ ID NO:94 and wherein said light chain variable region comprises a variable light (VL) CDR1 that has the amino acid sequence of SEQ ID NO:95, a VL CDR2 that has the amino acid sequence of SEQ ID NO:96 and a VL CDR3 that has the amino acid sequence of SEQ ID NO:97.

34. The antibody of any one of claims 1 to 18, 29, 30 or 31, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:112, a VH CDR2 that has the amino acid sequence of SEQ ID NO:113 and a VH CDR3 that has the amino acid sequence of SEQ ID NO: 114, or sequences substantially homologous thereto, and/or wherein said light chain variable region comprises a variable light (VL) CDR1 that has the amino acid sequence of SEQ ID NO:115, a VL CDR2 that has the amino acid sequence of SEQ ID NO:116 and a VL CDR3 that has the amino acid sequence of SEQ ID NO: 117, or sequences substantially homologous thereto,
wherein said substantially homologous sequences are sequences containing 1, 2 or 3 amino acid substitutions compared to the given CDR sequences, or said substantially homologous sequence is a sequence containing conservative amino acid substitutions of the given CDR sequences.

35. The antibody of any one of claims 1 to 18, 29, 30, 31 or 34, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:112, a VH CDR2 that has the amino acid sequence of SEQ ID NO:113 and a VH CDR3 that has the amino acid sequence of SEQ ID NO: 114 and wherein said light chain variable region comprises a variable light (VL) CDR1 that has the amino acid sequence of SEQ ID NO: 115, a VL CDR2 that has the amino acid sequence of SEQ ID NO: 116 and a VL CDR3 that has the amino acid sequence of SEQ ID NO:117.

36. The antibody of any one of claims 1 to 18, 29 or 30, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:132, a VH CDR2 that has the amino acid sequence of SEQ ID NO:133 and a VH CDR3 that has the amino acid sequence of SEQ ID NO:134, or sequences substantially homologous thereto, and/or wherein said light chain variable region comprises a variable light (VL) CDR1 that has the amino acid sequence of SEQ ID NO:135, a VL CDR2 that has the amino acid sequence of SEQ ID NO:136 and a VL CDR3 that has the amino acid sequence of SEQ ID NO:137, or sequences substantially homologous thereto,
wherein said substantially homologous sequences are sequences containing 1, 2 or 3 amino acid substitutions compared to the given CDR sequences, or said substantially homologous sequence is a sequence containing conservative amino acid substitutions of the given CDR sequences.

37. The antibody of any one of claims 1 to 18, 29, 30 or 36, wherein said antibody comprises at least one heavy chain variable region that comprises three CDRs and at least one light chain variable region that comprises three CDRs, wherein said heavy chain variable region comprises a variable heavy (VH) CDR1 that has the amino acid sequence of SEQ ID NO:132, a VH CDR2 that has the amino acid sequence of SEQ ID NO:133 and a VH CDR3 that has the amino acid sequence of SEQ ID NO:134 and wherein said light chain variable region comprises a variable light (VL) CDR1 that has the amino acid sequence of SEQ ID NO:135, a VL CDR2 that has the amino acid sequence of SEQ ID NO:136 and a VL CDR3 that has the amino acid sequence of SEQ ID NO:137.

38. The antibody of claim 29, wherein said antibody comprises a VL CDR1, VL CDR2, a VL CDR3 and a VH CDR3 that have amino acid sequences as defined together in any one claims 30 or 32 to 37.

39. The antibody of any one of claims 1 to 18 or 29 to 38, wherein said antibody comprises at least one heavy chain variable region that comprises three CDRs and at least one light chain variable region that comprises three CDRs, wherein
(i) the light chain variable region has the amino acid sequence of SEQ ID NO:91, or a sequence having at least 80% sequence identity thereto and/or wherein the heavy chain variable region has the amino acid sequence of SEQ ID NO:90, or a sequence having at least 80% sequence identity thereto;
(ii) the light chain variable region has the amino acid sequence of SEQ ID NO: 111, or a sequence having at least 80% sequence identity thereto and/or wherein the heavy chain variable region has the amino acid sequence of SEQ ID NO:110, or a sequence having at least 80% sequence identity thereto; or
(iii) the light chain variable region has the amino acid sequence of SEQ ID NO:131, or a sequence having at least 80% sequence identity thereto and/or wherein the heavy chain variable region has the amino acid sequence of SEQ ID NO:130, or a sequence having at least 80% sequence identity thereto.

40. The antibody of any one of claims 1 to 39, wherein said antibody is a monoclonal antibody or a polyclonal antibody.

41. The antibody of any one of claims 1 to 40, wherein said antibody is a monoclonal antibody.

42. The antibody of any one of claims 1 to 41, wherein said antibody is a whole antibody comprising an antibody constant region.

43. The antibody of any one of claims 1 to 42, wherein said antibody is an IgG antibody.

44. The antibody of any one of claims 1 to 41, wherein said antibody is an antigen binding fragment of an antibody.

45. The antibody of any one of claims 1 to 44, wherein said inhibition of activity is inhibition of GTPase activity and/or said inhibition of activity is characterised by the inhibition of ERK phosphorylation in cells that express said oncogenic mutant form of KRAS and/or said inhibition of activity is characterised by an increase in apoptosis in cells that express said oncogenic mutant form of KRAS and/or said inhibition of activity is characterised by an increase in necrosis in cells that express said oncogenic mutant form of KRAS.

46. A composition comprising an antibody of any one of claims 1 to 45 and a diluent, carrier or excipient, preferably a pharmaceutically acceptable diluent, carrier or excipient.

47. A nucleic acid molecule comprising a nucleotide sequence that encodes an antibody of any one of claims 1 to 45, or a set of nucleic acid molecules each comprising a nucleotide sequence, wherein said set of nucleic acid molecules together encode an antibody of any one of claims 1 to 45.

48. A method of producing an antibody according to any one of claims 1 to 45, comprising the steps of:
(i) culturing a host cell comprising (i) one or more nucleic acid molecules encoding an antibody according to any one of claims 1 to 45 or (ii) a set of nucleic acid molecules each comprising a nucleotide sequence, wherein said set of nucleic acid molecules together encode an antibody of any one of claims 1 to 45, or (iii) one or more recombinant expression vectors comprising one or more of said nucleic acid molecules, under conditions suitable for the expression of the encoded antibody; and
(ii) isolating or obtaining the antibody from the host cell or from the growth medium/supernatant.

49. An antibody as defined in any one of claims 1 to 45 for use in therapy.

50. An antibody as defined in any one of claims 1 to 45 for use in the treatment of cancer.

51. A method of treating cancer, said method comprising administering to a patient in need thereof a therapeutically effective amount of an antibody as defined in any one of claims 1 to 45.

52. Use of an antibody as defined in any one of claims 1 to 45 in the manufacture of a medicament for use in therapy.

53. The use according to claim 52, wherein said therapy is the treatment of cancer.

54. An isolated peptide, wherein said isolated peptide is as defined in any one of claims 9 to 17.

55. A conjugate comprising an isolated peptide as defined in any one of claims 9 to 17 and a peptide carrier, preferably said peptide carrier is keyhole limpet hemocyanin (KLH).
